(19) United States
(12) Patent Application Publication
(10) Pub. No.: US 2017/0168041 A1
(43) Pub. Date: Jun. 15, 2017

(54) Polymers And Oligomers With Aggregation-Induced Emission Characteristics For Imaging And Image-Guided Therapy

(71) Applicants: National University of Singapore, Singapore (SG); The Hong Kong University of Science and Technology, Kowloon, Hong Kong (CN)

(72) Inventors: Bin Liu, Singapore (SG); Youyong Yuan, Singapore (SG); Guangxue Feng, Singapore (SG); Ben Zhong Tang, Kowloon, Hong Kong (CN); Wei Qin, Kowloon, Hong Kong (CN); Chongjing Zhang, Singapore (SG); Shidang Xu, Singapore (SG)

(73) Assignees: National University of Singapore, Singapore (SG); The Hong Kong University of Science and Technology, Kowloon, Hong Kong (CN)

(21) Appl. No.: 15/305,203

(22) PCT Filed: Apr. 24, 2015
(86) PCT No.: PCT/SG15/00123

§ 371 Date: Oct. 19, 2016

Related U.S. Application Data
(60) Provisional application No. 61/984,459, filed on Apr. 25, 2014.

(51) Int. Cl. G01N 33/50; A61K 41/00; C07D 309/34; C07C 255/43; C09K 11/07; C07D 285/14; G01N 33/58; A61K 9/48

(52) U.S. Cl. CPC G01N 33/5011; G01N 33/582; A61K 41/0042; A61K 9/4866; C07C 255/43; C09K 11/07; C07D 285/14; C07D 309/34; A61K 2123/00; A61K 2121/00; C09K 2211/1088; C09K 2211/1051; C09K 2211/1014; C09K 2211/1007

(57) ABSTRACT

A fluorophore or conjugated polymer with aggregation-induced emission characteristics useful for drug tracking and delivery, identification and labeling of biological subjects, such as cells or parts of a cell, as well as for imaging, and image-guided photodynamic therapy are described herein.

### RELATED APPLICATION

This application claims the benefit of U.S. Provisional Application No. 61/984,459, filed on Apr. 25, 2014. The entire teachings of the above application are incorporated herein by reference.

### BACKGROUND OF THE INVENTION

During the past decades, fluorescence bioimaging has been extensively utilized in various biological science researches, such, as programmed cell death, cell organelle labelling, apoptosis, and cell lineage commitment. As compared to other imaging modalities, including positron emission imaging (PET), magnetic resonance imaging, single photon emission computing tomography, fluorescence utilizing readily available and biocompatible reagents is capable of producing high resolution images at sub-cellular levels, making the study of cell-cell interaction possible and gaining unique insights in immunology and biology. Among these biological studies, the continuous non-invasive active cell tracing by fluorescence over a long period of time is pivotal to extract critical spatiotemporal cellular information of physiological displacement, translocation and cell fate of cancer and stem cell. The information facilitates the understanding of cancer or stem cell development and intervention, providing insights for basic oncological researches and development of preclinical cell based therapies and immunotherapy.

Since its initial inception as a potent cell labelling agent, engineered expression of green fluorescent protein (GFP) and its variants have dominated the biological science field of cell transplantation and tracing experiments. This approach capitalizes on the cells innate machinery to produce proteins and requires the reporter gene to be transfected into the cells and subsequently translated into fluorescent proteins. Although viral transduction by integration of GFP gene into cell genome can result in stable GFP expression and be useful for long term tracing purpose, it suffers from high cost and safety issues due to the introduction of random insertional mutation at integration sites. Consequently, nonviral plasmid transfection using a wide range of biomaterials has been explored to circumvent the safety issues by intentionally avoiding the genomic integration but expressing the GFP plasmid directly from the cytoplasm. While this works well for short-lived experiments in the time scale of days, the plasmid is quickly lost with a correlated drop in fluorescence. In addition, the non-viral method presents low transfection efficiency which largely varies with the cell type, primary cell lines, mesenchymal stem cells are often refractory to non-viral transfection. Moreover, all protein expression starts with a convoluted and time-consuming transcriptional, translational and post-translationally regulated process and is subject to ubiquitination and proteosomal degradation; resulting in an inconsistent and sometimes even cyclical net amount of fluorescent signal even when actual intracellular plasmid concentration is high.

In stark contrast, direct cell labelling by organic or inorganic nanomaterials is fairly straightforward and does not involve genetic modification of the cells. However, current available fluorescence probes suffer from serious drawbacks. For example, quantum dots-based cell trackers contain toxic heavy metals, while fluorescent organic molecules suffered from a small Stokes shift, rapid photobleaching and cytoplasm leaking upon cell proliferation.

Recently, some theranostic (therapy combined with diagnostics) prodrug delivery systems have been developed for real-time monitoring of the active drug release by conjugating fluorescent dyes to the drug through a tumor-associated stimulated linker. The design strategy relies on drug release concomitant with fluorescence intensity change upon drug activation. Most of the systems reported so far are primarily focused on monitoring the drug activation after cellular uptake and only a single drug is used or monitored. In chemotherapy, the use of a single drug often fails to achieve complete cancer ablation due to the rapid development of drug resistance in tumor cells. As a consequence, non-cross resistant anticancer agents have been widely studied for efficient cancer therapy. Cisplatin (Pt(II)) and doxorubicin (DOX) are the two most effective anticancer drugs used in clinics for treating a variety of solid tumors. It is also reported that the co-administration of cisplatin and DOX will result in greatly enhanced therapeutic activities than the solely treatment and some of them have already been applied for clinical trials.

Polymeric nanoparticles (NPs) formed by self-assembly of amphiphilic block copolymers in aqueous solution have received broad attention as a promising vehicles for drug delivery. These systems exhibit many advantages for biomedical applications such as favorable biodistribution, long circulation, high therapeutic effects and low side effects of the drugs, which have been widely used for chemotherapy, gene therapy, photothermal therapy, photodynamic therapy (PDT) and so on. Among them, newly emerging PDT which based on the concept that photosensitizers can generate cytotoxic reactive oxygen species (ROS) capable of killing tumor cells when exposed to light of specific wavelength has gained increasingly attentions. Typically, the photosensitizers are loaded into the delivery system via hydrophobic-hydrophobic interaction. However, photosensitizers in these delivery systems could aggregate easily due to π-π interactions (such as the most widely used commercial PDT agents based on porphyrin), restating in a dramatic reduced ROS generation with reduced PDT efficiency.

### SUMMARY OF THE INVENTION

The invention pertains to compounds, polymers, and probes for visualization of biological subjects, such as cells, photodynamic therapy, drug and gene delivery; methods for assessing the conversion of a prodrug, treatment of cancer through combination chemotherapy and photodynamic therapy, and designing and screening photo sensitizer compounds for photodynamic therapy. The compounds, uses, and methods of the present invention are advantageous over the prior art because they provide venues for efficient and effective drug and gene delivery, as well as allow for selective photoexcitation for nuanced imaging of biological targets.

In a first aspect, an example embodiment of the present invention is a fluorophore having the structure of Formula (I):

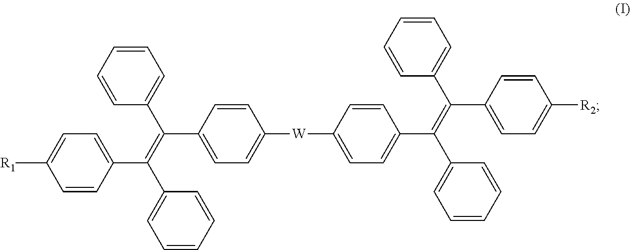

or a pharmaceutically acceptable salt thereof;
wherein W is a conjugated system;
R₁ or R₂ is H or CH₂X;
X is N₃, NH₂, COOH, —C≡CH, halo, —SH, maleimide or OH, which allows further conjugation to different chemicals and biomolecules and the fluorophore exhibits aggregation-induced emission properties.

In another embodiment of the first aspect, the conjugated system comprises one or more aromatic rings, one or more heteroaromatic rings, one or more alkenes, one or more heteroatoms comprising a p-orbital, or a combination thereof.

In another embodiment of the first aspect, the present invention is a fluorophore having the structure of Formula (II):

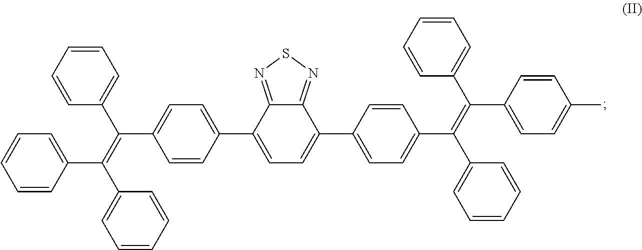

or a pharmaceutically acceptable salt thereof.

In another embodiment of the first aspect, the present invention is a fluorophore having the structure of Formula (III):

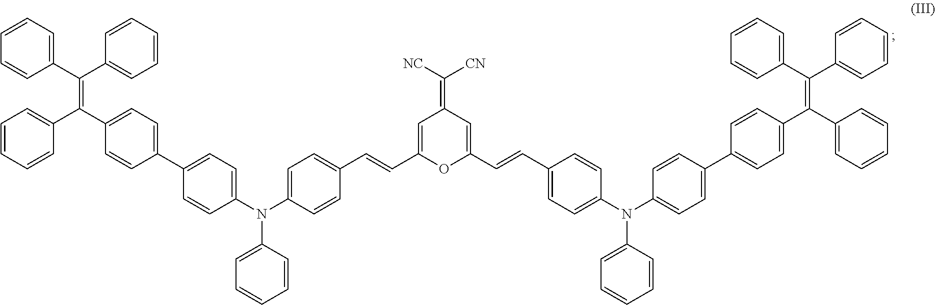 [SMILES: N#CC(C#N)=C1C=C(/C=C/c2ccc(N(c3ccccc3)c3ccc(-c4ccc(C(=C(c5ccccc5)c5ccccc5)c5ccccc5)cc4)cc3)cc2)OC(/C=C/c2ccc(N(c3ccccc3)c3ccc(-c4ccc(C(=C(c5ccccc5)c5ccccc5)c5ccccc5)cc4)cc3)cc2)=C1]

or a pharmaceutically acceptable salt thereof.

In another embodiment of the first aspect, the present invention is a fluorophore having the structure of Formula (VI):

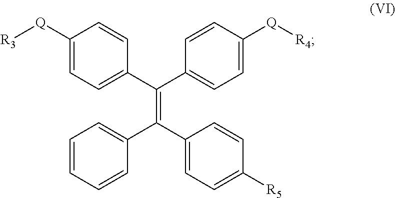

or a pharmaceutically acceptable salt thereof

wherein
Q is O, N(C₁-C₃)alkyl, or Si;
R₃ and R₄ are H, (C₁-C₃) alkyl optionally substituted with one or more substitutents selected from halo, amino, N₃, or PPh₃, 5-10 atom heterocyclyl, —C(O)C₂-C₆ alkynyl or

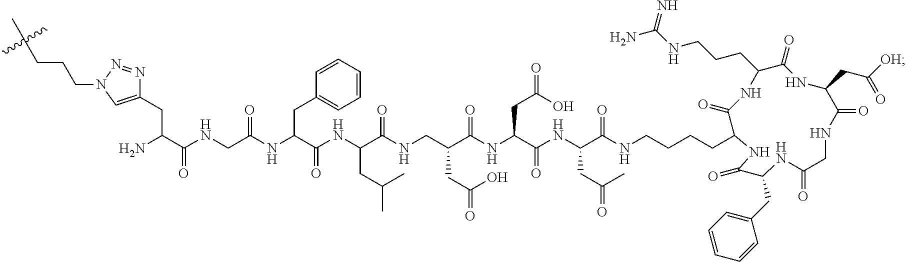 [SMILES: *CCCn1cc(CC(N)C(=O)NCC(=O)NC(Cc2ccccc2)C(=O)NC(CC(C)C)C(=O)NC[C@@H](CC(=O)O)C(=O)N[C@@H](CC(=O)O)C(=O)N[C@@H](CC(C)=O)C(=O)NCCCCC2NC(=O)[C@@H](Cc3ccccc3)NC(=O)CNC(=O)[C@H](CC(=O)O)NC(=O)C(CCCNC(=N)N)NC2=O)nn1]

### R₅ is

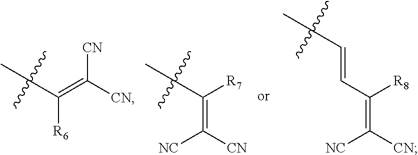

R₆ is C₁-C₆ alkyl;

R₇ is (C₁-C₆)alkyl or (C₂-C₆)alkenyl, optionally substituted with aryl or heteroaryl, each further optionally substituted with —O—(C₁-C₆) alkylamino; and the fluorophore exhibits aggregation-induced emission properties.

In another embodiment of the first aspect, the present invention is a fluorophore having the structure of Formula (VI):

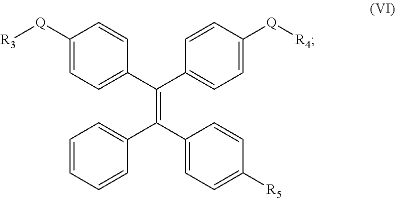

or a pharmaceutically acceptable salt thereof

wherein
Q is O or N(C₁-C₃)alkyl;
R₃ and R₄ are H, (C₁-C₃) alkyl optionally substituted with one or more substitutents selected from halo, amino, N₃, or PPh₃, 5-10 atom heterocyclyl, or —C(O)C₂-C₆ alkynyl;

### R₅ is

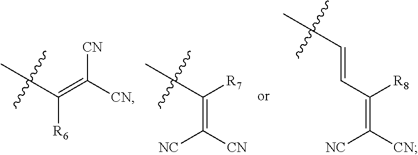

R₆ is C₁-C₆ alkyl;

R₇ is (C₁-C₆)alkyl or (C₂-C₆)alkenyl, optionally substituted with aryl or heteroaryl, each further optionally substituted with —O—(C₁-C₆) alkylamino; and
the fluorophore exhibits aggregation-induced emission properties.

In another embodiment of the first aspect, the present invention does not include:

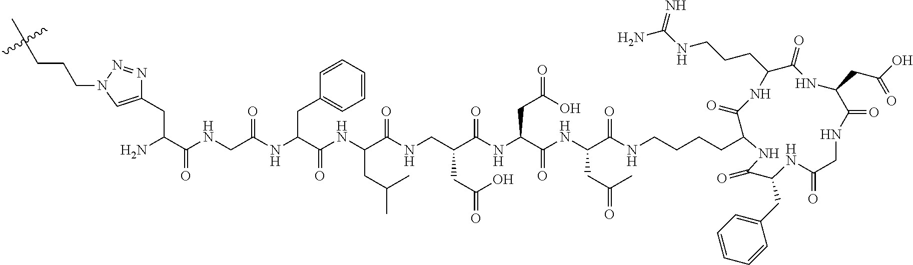 [SMILES: *CCCn1cc(CC(N)C(=O)NCC(=O)NC(Cc2ccccc2)C(=O)NC(CC(C)C)C(=O)NC[C@@H](CC(=O)O)C(=O)N[C@@H](CC(=O)O)C(=O)N[C@@H](CC(C)=O)C(=O)NCCCCC2NC(=O)[C@@H](Cc3ccccc3)NC(=O)CNC(=O)[C@H](CC(=O)O)NC(=O)C(CCCNC(=N)N)NC2=O)nn1]

In another embodiment of the first aspect, the present invention is a fluorophore having the structure of Formula (VII):

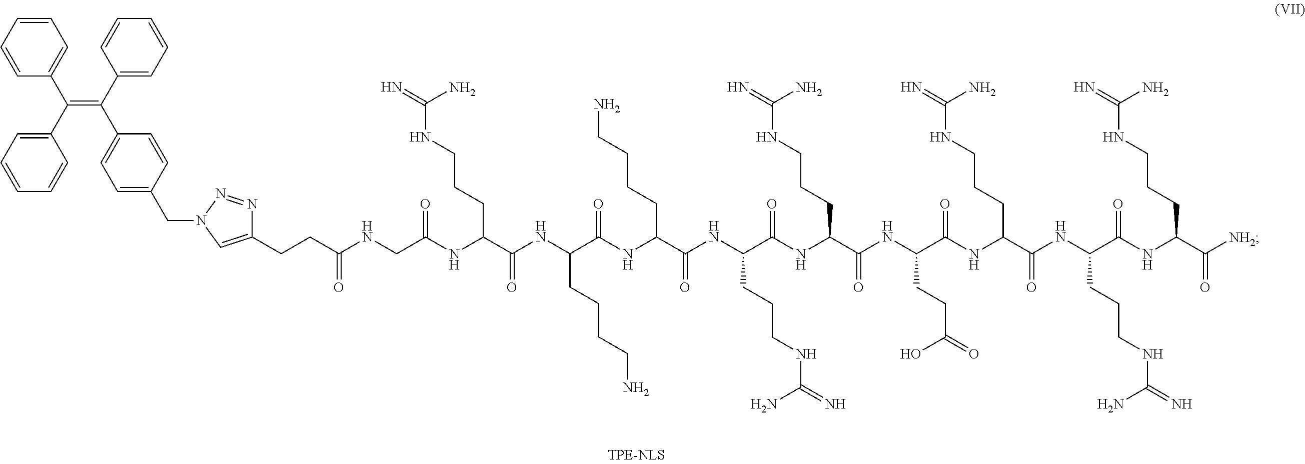 [SMILES: N=C(N)NCCCC(NC(=O)CNC(=O)CCc1cn(Cc2ccc(C(=C(c3ccccc3)c3ccccc3)c3ccccc3)cc2)nn1)C(=O)NC(CCCCN)C(=O)NC(CCCCN)C(=O)N[C@@H](CCCNC(=N)N)C(=O)N[C@@H](CCCNC(=N)N)C(=O)N[C@@H](CCC(=O)O)C(=O)NC(CCCNC(=N)N)C(=O)N[C@@H](CCCNC(=N)N)C(=O)N[C@@H](CCCNC(=N)N)C(N)=O]

or a pharmaceutically acceptable salt thereof.

In another embodiment of the first aspect, the present invention is a fluorophore having the structure of Formula (VIII):

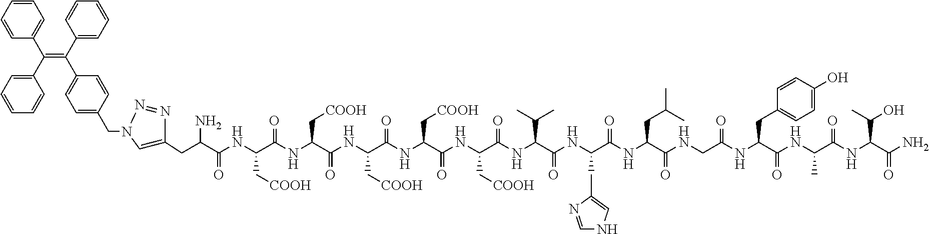 [SMILES: CC(C)C[C@H](NC(=O)[C@H](Cc1c[nH]cn1)NC(=O)[C@@H](NC(=O)[C@H](CC(=O)O)NC(=O)[C@H](CC(=O)O)NC(=O)[C@H](CC(=O)O)NC(=O)[C@H](CC(=O)O)NC(=O)[C@H](CC(=O)O)NC(=O)C(N)Cc1cn(Cc2ccc(C(=C(c3ccccc3)c3ccccc3)c3ccccc3)cc2)nn1)C(C)C)C(=O)NCC(=O)N[C@@H](Cc1ccc(O)cc1)C(=O)N[C@@H](C)C(=O)N[C@H](C(N)=O)C(C)O]

In another embodiment of the first aspect, the present invention is a fluorophore having the structure of Formula (VIII):

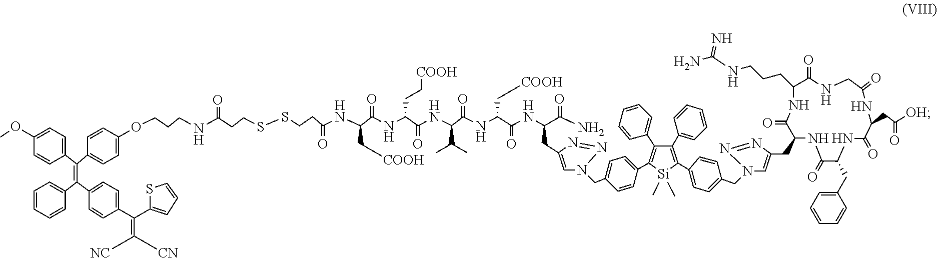 [SMILES: COc1ccc(/C(=C(\c2ccccc2)c2ccc(C(=C(C#N)C#N)c3cccs3)cc2)c2ccc(OCCCNC(=O)CCSSCCC(=O)N[C@H](CC(=O)O)C(=O)N[C@H](CCC(=O)O)C(=O)N[C@@H](C(=O)N[C@H](CC(=O)O)C(=O)N[C@H](Cc3cn(Cc4ccc(C5=C(c6ccccc6)C(c6ccccc6)=C(c6ccc(Cn7cc(C[C@@H]8NC(=O)[C@@H](Cc9ccccc9)NC(=O)[C@H](CC(=O)O)NC(=O)CNC(=O)C(CCCNC(=N)N)NC8=O)nn7)cc6)[Si]5(C)C)cc4)nn3)C(N)=O)C(C)C)cc2)cc1]

or a pharmaceutically acceptable salt thereof.

In another embodiment of the first aspect, the present invention is a fluorophore having the structure of Formula (IX):

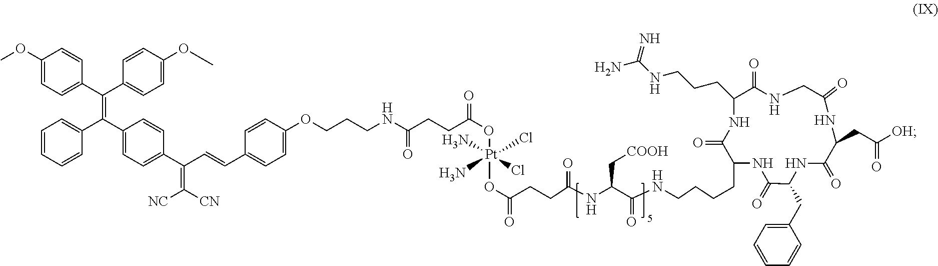

or a pharmaceutically acceptable salt thereof.

In another embodiment of the first aspect, the present invention is a fluorophore having the structure of Formula (X):

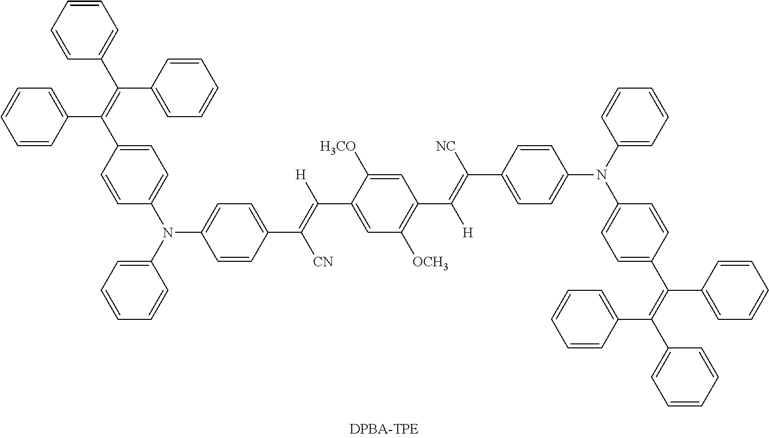 [SMILES: COc1cc(/C=C(\C#N)c2ccc(N(c3ccccc3)c3ccc(C(=C(c4ccccc4)c4ccccc4)c4ccccc4)cc3)cc2)c(OC)cc1/C=C(\C#N)c1ccc(N(c2ccccc2)c2ccc(C(=C(c3ccccc3)c3ccccc3)c3ccccc3)cc2)cc1]

or a pharmaceutically acceptable salt thereof.

In another embodiment of the first aspect, the fluorophore is encapsulated into a biocompatible matrix; wherein the biocompatible matrix comprises lipids (e.g. DSPE-PEG), polyethylene glycol, chitosan, polyvinyl alcohol, poly(2-hydroxyethylmethacrylate) or bovine serum albumin;

wherein polyethylene glycol, chitosan, polyvinyl alcohol, poly(2-hydroxyethylmethacrylate) or bovine serum albumin is optionally functionalized by one or more lipids, maleimide, hydroxyl, amine, carboxyl, sulfhydryl or a combination thereof.

In another embodiment of the first aspect, an outer surface of the biocompatible matrix is functionalized with a cell penetrating peptide comprising an amino acid residue sequence of Arg-Lys-Lys-Arg-Arg-Gln-Arg-Arg-Arg-Cys (SEQ ID NO: 1); Arg-Arg-Arg-Arg-Arg-Arg-Arg-Arg (SEQ ID NO: 2); Lys-Arg-Pro-Ala-Ala-Thr-Lys-Lys-Ala-Gly-Gln-Ala-Lys-Lys-Lys-Leu (SEQ ID NO: 3); and Gly-Leu-Ala-Phe-Leu-Gly-Phe-Leu-Gly-Ala-Ala-Gly-Ser-Thr-Met-Gly-Ala-Trp-Ser-Gln-Pro-Lys-Lys-Lys-Arg-Lys-Val (SEQ ID NO: 4) Gly-Arg-Lys-Lys-Arg-Arg-Gln-Arg-Arg-Arg (SEQ ID NO: 5); Val-His-Leu-Gly-Tyr-Ala-Thr (SEQ ID NO: 8) or a pharmaceutically acceptable salt thereof.

In a second aspect, the present invention is the use of any one of the fluorophores described above in the visualization of a cell or bacteria or any other organism.

In an example embodiment of the second aspect, the present invention is the use of any one of the fluorophores described above in the photodynamic therapy of a cell or bacteria or any other organism.

In an example embodiment of the second aspect, the present invention is the use of any one of the fluorophores described above in imaging and image-guided photodynamic therapy of a cell bacteria or any other organism.

In an example embodiment of the second aspect, the present invention is the use of any one of the fluorphores described above in the visualization of an organelle of a cell.

In an example embodiment of the second aspect, the organelle is a mitochondria.

In a third aspect, the present invention is a chemical composition, comprising: a target recognition motif, a fluorophore, a linking moiety and a chemotherapeutic drug, wherein the target recognition motif, the fluorophore, the linking moiety and the chemotherapeutic drug are linked by covalent linkages in a linear array; the target recognition motif is at a terminal end of the linear array; and further wherein the fluorophore exhibits aggregation-induced emission properties and comprises a tetraphenylethylene optionally substituted with H, OH, or O(C₁-C₆)alkyl.

In another embodiment of the third aspect, the linking moiety is a prodrug, chemical responsive, ROS responsive, or pH responsive. The linking moiety is intended to break upon exposure to external stimuli.

In another embodiment of the third aspect, the prodrug is a platinum (IV) complex.

In another embodiment of the third aspect, the target recognition motif has an affinity for a cell membrane receptor.

In another embodiment of the third aspect, the target recognition motif is a cyclic(Arg-Gly-Asp) residue having an affinity for integrin 43.

In another embodiment of the third aspect, the target recognition motif is a Val-His-Leu-Gly-Tyr-Ala-Thr (SEQ ID NO: 8) residue having an affinity for HT-29 cells.

In another embodiment of the third aspect, the chemotherapeutic drug is doxorubicin.

In another embodiment of the third aspect, the composition has the structure of Formula (IV):

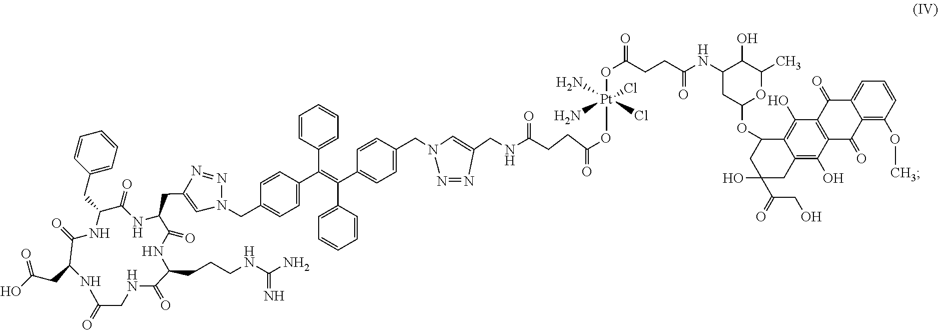

or a pharmaceutically acceptable salt thereof.

In a fourth aspect, the present invention is a method for assessing the conversion of a prodrug into its active form, comprising: a) incubating a biological sample with a composition of the third aspect under conditions sufficient to form an incubated mixture; and b) analyzing the fluorescence of the incubated mixture of step a), wherein a change in fluorescence signal as compared to the fluorescence signal of the composition of any one of the compositions described above not in the presence of the biological sample is indicative of the conversion of the prodrug into its active form.

In another embodiment of the fourth aspect, the method is conducted in a live cell.

In another embodiment of the fourth aspect, the step of incubating further comprises incubating the biological sample with ascorbic acid or glutathione.

In a fifth aspect, the present invention is a conjugated polymer of Formula (V):

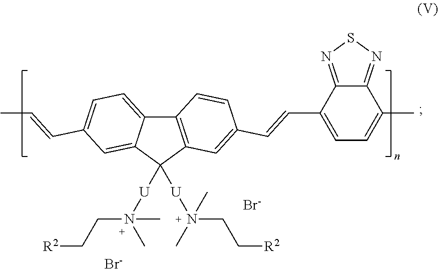

or a salt thereof, wherein:
U is (C₁-C₂₀)alkyl or (CH₂CH₂O)₁₋₂₀;

### R² is

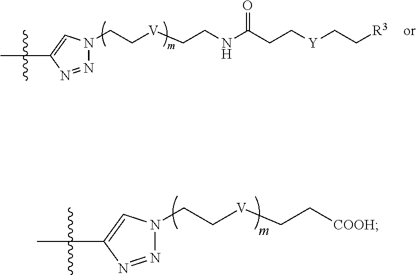

### V is O or NH;

### Y is

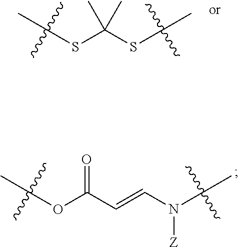

Z is H or (C₁-C₆)alkyl;
each R³ is independently —COOH or —CO—B;
B is a chemotherapeutic drug;
n is an integer from 5-115; and
m is an integer from 5-115.

In an example embodiment of the fifth aspect, at least one R³ is —CO—B.

In an example embodiment of the fifth aspect, the chemotherapeutic drug is doxorubicin, paclitaxel, melphalan, camptothecin, or gemcitabine.

In an example embodiment of the fifth aspect, the conjugated polymer is a conjugated polymer-based nanoparticle.

In an example embodiment of the fifth aspect, an outer surface of the nanoparticle is functionalized by a target-recognition motif.

In an example embodiment of the fifth aspect, the target recognition motif has an affinity for a cell membrane receptor.

In an example embodiment of the fifth aspect, the target recognition motif is a cyclic(Arg-Gly-Asp) residue having an affinity for integrin αᵥβ₃.

In an example embodiment of the fifth aspect, R² is

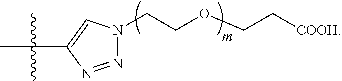

In an example embodiment of the fifth aspect, the conjugated polymer is a conjugated polymer-based nanoparticle.

In an example embodiment of the fifth aspect, a chemotherapeutic drug is encapsulated into the conjugated polymer-based nanoparticle.

In an example embodiment of the fifth aspect, the chemotherapeutic drug is paclitaxel.

In an example embodiment of the fifth aspect, an outer surface of the nanoparticle is functionalized by a target-recognition motif.

In an example embodiment of the fifth aspect, the target recognition motif has an affinity for a cell membrane receptor.

In an example embodiment of the fifth aspect, the target recognition motif is a cyclic(Arg-Gly-Asp) residue having an affinity for integrin αᵥβ₃.

In a sixth aspect, the present invention is the use of the conjugated polymer-based nanoparticle recited above in imaging-guided chemotherapy and photodynamic therapy.

In a seventh aspect, the present invention is a method for the treatment of cancer through combination chemotherapy and photodynamic therapy, comprising: a) incubating a biological sample thought to contain cancer cells with the conjugated polymer-based nanoparticle of any one of the compositions recited above under conditions sufficient to form an incubated mixture, wherein at least one R³ is —CO—B; and b) irradiating the incubated mixture with a light of a wavelength sufficient to generate a reactive oxygen species, wherein the reactive oxygen species reacts with the conjugated polymer to convert the chemotherapeutic drug into an active form and further wherein the reactive oxygen species activates the conjugated polymer to serve as a photosensitizer.

In an example embodiment of the seventh aspect, the method further comprises visualizing the irradiated mixture by fluorescence, wherein a change in fluorescence signal of the irradiated mixture, as compared to the fluorescence signal of the conjugated polymer-based nanoparticle of any one of compositions recited above prior to incubation is indicative of conversion of the chemotherapeutic drug into an active form.

In an example embodiment of the seventh aspect, the method further comprises determining cellular uptake of the conjugated polymer-based nanoparticle by fluorescence imaging.

In an example embodiment of the seventh aspect, the step of determining cellular uptake of the conjugated polymer-based nanoparticle is quantitative.

In an eighth aspect, the present invention is a fluorophore having the structure of Formula (XI):

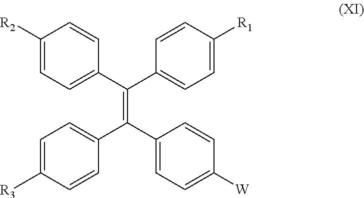

or a pharmaceutically acceptable salt thereof;
wherein W is a conjugated system;
R₁ and R₂ are H, OH, N(C₁-C₃)alkyl or O(C₁-C₆) alkyl optionally substituted with one or more substituents selected from halo, amino, PPh₃, 5-10 atom heterocycyl, N₃, —C(O)(C₂-C₆)alkynyl or X;
R₃ is H, OH, N(C₁-C₃)alkyl or O(C₁-C₆) alkyl optionally substituted with one or more substituents selected from halo, amino, PPh₃, 5-10 atom heterocycyl, N₃, —C(O)(C₂-C₆)alkynyl, X or W;
X is a moiety comprising a linking moiety, a plurality of hydrophilic peptides, a target recognition motif and optionally TPE2; and
the fluorophore exhibits aggregation-induced emission properties.

In an example embodiment of the eighth aspect, the conjugated system comprises one or more aromatic rings, one or more heteroaromatic rings, one or more alkenes, one or more heteroatoms comprising a p-orbital, or a combination thereof.

In an example embodiment of the eighth aspect, the conjugated system is:

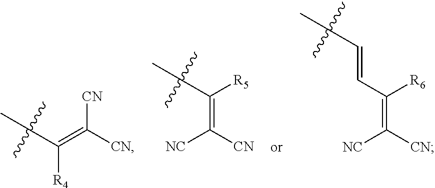

R₄ is (C₁-C₆) alkyl optionally substituted with N₃, amino, (C₁-C₃)alkynyl, —C(O)OH, halo, —SH, maleimide or OH;
R₅ is aryl, heteroaryl, (C₁-C₆) alkyl or (C₂-C₆) alkenyl optionally substituted with N₃, amino, (C₁-C₃)alkynyl, —C(O)OH, halo, —SH, maleimide, OH, aryl or heteroaryl, each further optionally substituted with —O—(C₁-C₆) alkylamino; and
R₆ is aryl or heteroaryl.

In an example embodiment of the eighth aspect, the linking moiety comprises a chemical bond that breaks upon exposure to an external stimulus. In an example embodiment of the eighth aspect, the linker is

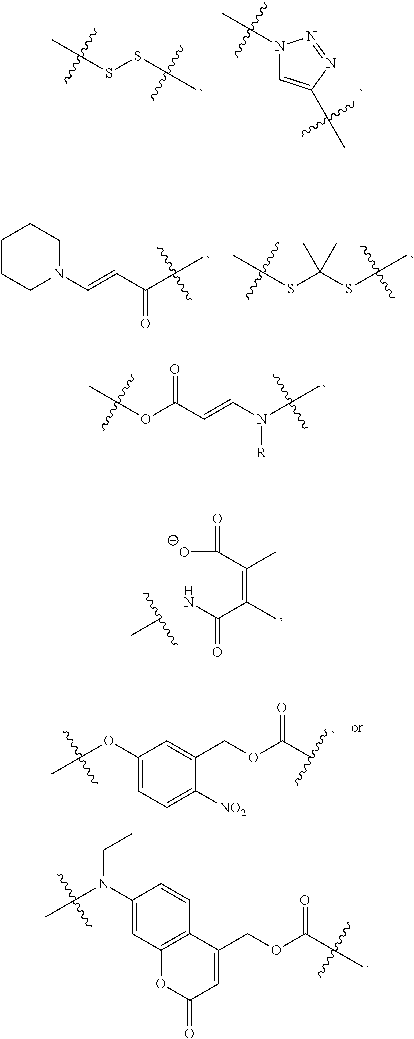

In an example embodiment of the eighth aspect, the target recognition motif specifically binds to an biological target.

In an example embodiment of the eighth aspect, the biological target is a protein, a surface biomarker, a cell surface marker, or a bacteria surface marker.

In an example embodiment of the eighth aspect, the target recognition motif is a cyclic(Arg-Gly-Asp) residue having an affinity for integrin αᵥβ₃.

In an example embodiment of the eighth aspect, the conjugated system is

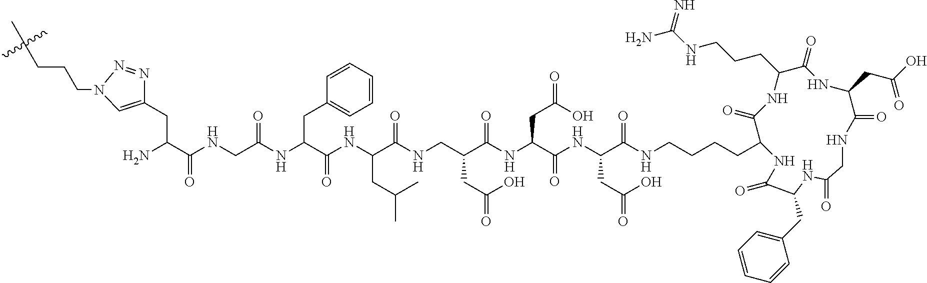 [SMILES: *CCCn1cc(CC(N)C(=O)NCC(=O)NC(Cc2ccccc2)C(=O)NC(CC(C)C)C(=O)NC[C@@H](CC(=O)O)C(=O)N[C@@H](CC(=O)O)C(=O)N[C@@H](CC(=O)O)C(=O)NCCCCC2NC(=O)[C@@H](Cc3ccccc3)NC(=O)CNC(=O)[C@H](CC(=O)O)NC(=O)C(CCCNC(=N)N)NC2=O)nn1]

In an example embodiment of the eighth aspect, the fluorophore does not include

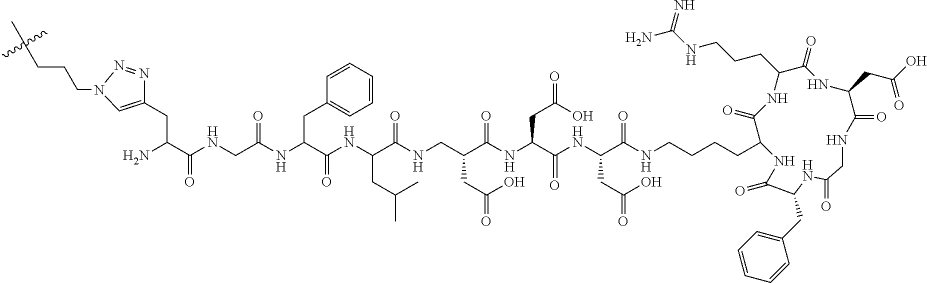 [SMILES: *CCCn1cc(CC(N)C(=O)NCC(=O)NC(Cc2ccccc2)C(=O)NC(CC(C)C)C(=O)NC[C@@H](CC(=O)O)C(=O)N[C@@H](CC(=O)O)C(=O)N[C@@H](CC(=O)O)C(=O)NCCCCC2NC(=O)[C@@H](Cc3ccccc3)NC(=O)CNC(=O)[C@H](CC(=O)O)NC(=O)C(CCCNC(=N)N)NC2=O)nn1]

In a ninth aspect, the present invention is a probe for visualizing a biological subject, the probe comprising a fluorophore, a linking moiety and a plurality of peptides, wherein the fluorophore, the linking moiety and the plurality of peptides are linked by covalent linkages in a linear array; and

further wherein the fluorophore exhibits aggregation-induced emission properties and comprises a tetraphenylethylene optionally substituted with H, OH, O(C₁-C₆)alkyl, aryl, heteroaryl, or (C₂-C₆) alkenyl further optionally substituted with —CN.

In an example embodiment of the ninth aspect, the probe has the structure of Formula (VII):

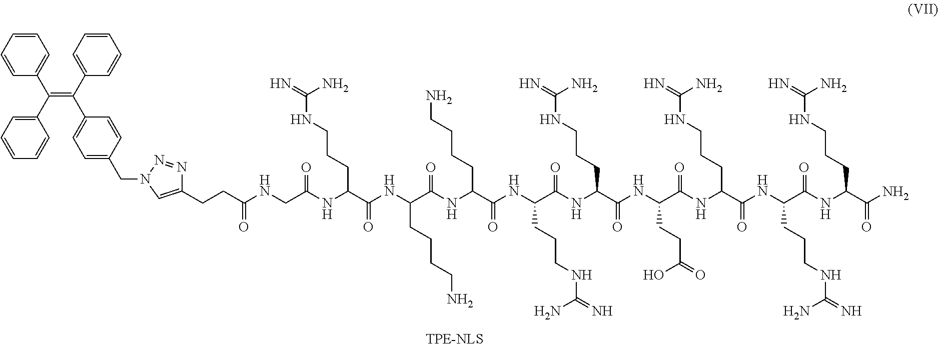 [SMILES: N=C(N)NCCCC(NC(=O)CNC(=O)CCc1cn(Cc2ccc(C(=C(c3ccccc3)c3ccccc3)c3ccccc3)cc2)nn1)C(=O)NC(CCCCN)C(=O)NC(CCCCN)C(=O)N[C@@H](CCCNC(=N)N)C(=O)N[C@@H](CCCNC(=N)N)C(=O)N[C@@H](CCC(=O)O)C(=O)NC(CCCNC(=N)N)C(=O)N[C@@H](CCCNC(=N)N)C(=O)N[C@@H](CCCNC(=N)N)C(N)=O]

or a pharmaceutically acceptable salt thereof.

In an example embodiment of the ninth aspect, the probe has structure of Formula (VIII):

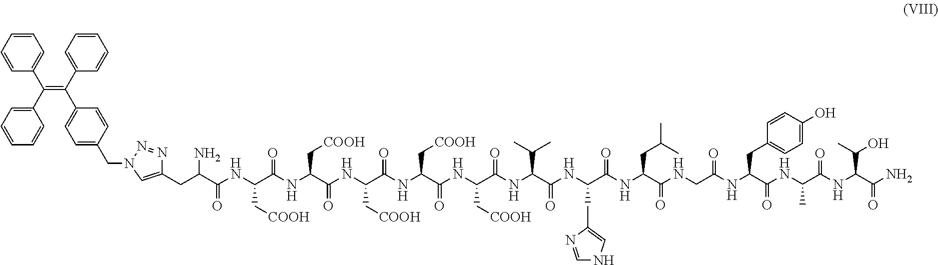 [SMILES: CC(C)C[C@H](NC(=O)[C@H](Cc1c[nH]cn1)NC(=O)[C@@H](NC(=O)[C@H](CC(=O)O)NC(=O)[C@H](CC(=O)O)NC(=O)[C@H](CC(=O)O)NC(=O)[C@H](CC(=O)O)NC(=O)[C@H](CC(=O)O)NC(=O)C(N)Cc1cn(Cc2ccc(C(=C(c3ccccc3)c3ccccc3)c3ccccc3)cc2)nn1)C(C)C)C(=O)NCC(=O)N[C@@H](Cc1ccc(O)cc1)C(=O)N[C@@H](C)C(=O)N[C@H](C(N)=O)C(C)O]

or a pharmaceutically acceptable salt thereof.

In a tenth aspect, the present invention is the use of the probes described above in the visualization of a biological subject including, for example, a cell or a bacterium.

In an example embodiment of the tenth aspect, the cell is a cancer cell.

In an example embodiment of the tenth aspect, the cell is an HT-29 cell.

In an eleventh aspect, the present invention is the use of the probes in the visualization of an organelle of a cell.

In an example embodiment of the eleventh aspect, the organelle is a mitochondria.

In a twelfth aspect, the present invention is the use of the probe in the image-guided photodynamic therapy a cell.

In a thirteenth aspect, the present invention is a polymer comprising a fluorophore, a linking moiety and an oligoethylenimine, wherein the fluorophore, the linking moiety and the oligoethylenimine are linked by covalent linkages in a linear array; and further wherein the fluorophore exhibits aggregation-induced emission properties and comprises a tetraphenylethylene optionally substituted with H, OH, O(C₁-C₆)alkyl, aryl, heteroaryl, or (C₂-C₆) alkenyl further optionally substituted with —CN.

In an example embodiment of the thirteenth aspect, the polymer has the structure of Formula (XII)

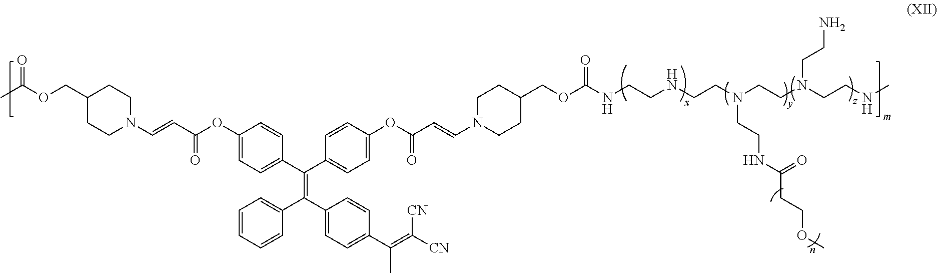

wherein m is an integer between 1 and 200, n is an integer between 5 and 400, and x+y+z is an integer between 5 and 10.

In a fourteenth aspect, the present invention is a method of delivering a target agent to a cell, the method comprising:

a) contacting the polymer with the target agent under conditions sufficient to form an agent-polymer particle;

b) incubating the cell with the agent-polymer particle under conditions sufficient to form an incubated mixture; and

b) irradiating the incubated mixture with a light of a wavelength sufficient to generate a reactive oxygen species, wherein the reactive oxygen species reacts with the agent-polymer particle to release the target agent from the agent-polymer particle into the cell.

In an example embodiment of the fourteenth aspect, the agent is DNA, RNA, SiRNA, or a drug.

In a fifteenth aspect, the present invention is a method for designing and screening a photosensitizer compound for photodynamic therapy, comprising:

a) selecting a class of compounds comprising a donor moiety and an acceptor moiety;

b) calculating, for a plurality of members of the class of compounds, values of the energy gap between the singlet and triplet excited states (ΔE_{ST});

c) identifying members of the class of compounds with ΔE_{ST} less than or equal to 1;

d) photoexciting the identified members of the class of compounds to generate singlet oxygen;

e) selecting the photosensitizer compound from the compounds of step (d) with the highest singlet oxygen quantum yield.

### BRIEF DESCRIPTION OF THE DRAWINGS

The foregoing will be apparent from the following more particular description of example embodiments of the invention, as illustrated in the accompanying drawings.

FIG. 1 illustrates a synthetic route for PPDC.

FIG. 2A illustrates a synthetic route to the functionalizable TPE derivative TPECM-2N3 and the bioprobe TPECM-2GFLGD3-cRGD.

FIG. 2B illustrates probe activation by cathepsin B with fluorescence “turn-on” and activated photoactivity to generate reactive oxygen species (ROS) upon irradiation with light.

FIGS. 3A-F are confocal images of A) MDA-MB-231 cells, B) MCF-7 cells, C) 293T cells, D) MDA-MB-231 cells pretreated with free cRGD, E) MDA-MB-231 cells pretreated with CA-074-Me, and F) MDA-MB-231 cells pretreated with both cRGD and CA-074-Me after incubation with the probe (5 mm) for 4 h. The blue fluorescence is from the cell nuclei dyed with 4′,6-diamidino-2-phenylindole (DAPI; Ex=405 nm; Em=430-470 nm), the red fluorescence is from the probe (Ex=405 nm; Em>560 nm). All images share the same scale bar (20 mm).

FIGS. 4A-C illustrates synthetic schemes for TPE compounds useful in the present invention.

FIG. 5 illustrates synthetic schemes for additional TPE compounds useful in the present invention.

FIG. 6 illustrates confocal images of HeLa cells after incubation with 2 μM TPECM-1TPP (A-D), TPECM-2TPP (F-I) and TPECM-2Br (K-N), co-stained with 100 nM Mito-tracker green. The green fluorescence is from Mito-tracker green, λₑₓ=488 nm and λₑₘ=520 nm±20 nm, the red fluorescence is from the probes, λₑₓ=405 nm, λₑₘ>560 nm long pass filter. All images share the same scale bar of 20 μm. Co-localization scatter plots for TPECM-1TPP (E), TPECM-2TPP (J) and TPECM-2Br (0) in mitochondria of HeLa cells.

FIG. 7 illustrates the mitochondrial morphology change of MDA-MB-231 cells after treatment with TPECM-1TPP (5 μM) under dark (A C) or light irradiation (0.1 W cm⁻², 8 min) (D-F). A and D are images from Mito-tracker green, λₑₓ=488 nm; λₑₘ=520 nm±20 nm. B and E are images from TPECM-1TPP, λₑₓ=405 nm; λₑₓ>560 nm long pass filter. C and F are overlay images from Mito-tracker green and TPECM-1TPP.

FIG. 8 is confocal fluorescence (A, D, G and J), bright field (B, E, H and K) and overlay fluorescence and bright field (C, F, I and L) images of PI stained HeLa cells after incubation of the cells without TPECM-2TPP (A, B and C), or with TPECM-2TPP (1 μM) in dark for 24 h (D, E and F) or with TPECM-2TPP (1 μM) for 3 h in dark followed by washing-away of the probe, white light irradiation (8 min, 0.10 W cm⁻²) and further incubation for 24 h (G, H and I) or with TPECM-2TPP (1 μM) for 3 h in dark followed by washing-away of the probe, pre-incubation with Vitamin C (100 μM, 15 min), white light irradiation (8 min, 0.10 W cm⁻²) and further incubation for 24 h (J, K and L).

FIG. 9 is a synthetic route to the ROS-responsive polymer useful in the present invention.

FIGS. 10A-F4 illustrate (A) CLSM images of HeLa cells stained with S-NPs/DNA (A1, Eₓ: 405 nm, Eₘ: >560 nm) and LysoTracker green (A2, Eₓ: 488 nm, Eₘ: 505-525 nm); (A3) overlay of the images A1 and A2; (A4) intensity profiles of region of interest (circled area in image A3). (B) CLSM images of HeLa cells incubated with S-NPs/YOYO-1-DNA complexes (B1) in dark, with light irradiation for (B2) 2 min, (B3) 5 min and (B4) 5 min in the presence of VC. Green: YOYO-1 fluorescence (Eₓ: 488 nm; Eₘ: 505-525 nm); Red: S-NPs fluorescence (Eₓ: 405 nm; Eₘ: >560 nm). Yellow: co-localization of red and green pixels. (C) Changes in co-localization ratios between the fluorescence of YOYO-1 and S-NPs after different treatment. (D, E) CLSM images of HeLa cells incubated with (D)S-NPs/YOYO-1-DNA pretreated with chloroquine (CQ), (E) inS-NPs/YOYO-1-DNA in dark (D1, E1) or with 5 min light irradiation (D2, E2). (F) CLSM images illustrating localization of YOYO-1-DNA after different treatments with further 4 h incubation. S-NPs/DNA in dark (F1), S-NPs/DNA with light irradiation (F2), S-NPs/DNA in the presence of VC with light irradiation (F3) and inS-NPs/DNA with light irradiation (F4). Green: YOYO-1 fluorescence (Eₓ: 488 nm; Eₘ: 505-525 nm); Red: nuclei living stained with DRAQ5 (Eₓ: 633 nm; Eₘ: >650 nm); Yellow: co-localization of red and green pixels. All images share the same scale bar of 10 μm.

FIG. 11 illustrates the synthetic route for TPE-NLS.

FIG. 12 illustrates the fluorescence intensity of 10 μM TPE-NLS upon addition of cellular components: dsDNA (A), histone (B) and nuclear lysate (C) at different concentrations in DMSO/1×PBS (1:99 v/v). λex=312 nm, λem=480 nm.

FIGS. 13A-B are a schematic illustration of the dual-targeted theranostic probe.

FIG. 14 illustrates a synthetic pathway for TPETP-NH₂.

FIG. 15 illustrates the reduction responsiveness of the TPETP-SS-DEVD-TPS-cRGD. (a) Normalized UV-vis absorption and PL spectra of TPETP in DMSO/water (v/v=1/199). (b) PL spectra of TPETP in DMSO/water mixtures at different water fractions (f_{w}). (c) PL spectra of TPETP and the probe in DMSO/PBS mixtures (v/v=1/199). Inset: the corresponding photographs taken under illumination of a UV lamp at 365 nm. (d) Time-dependent PL spectra of the probe (10 μM) incubated with GSH (0.1 mM). (e) Plot of PL intensity at 650 nm versus concentrations of the probe with the incubation of GSH (0.1 mM) for 75 min in DMSO/PBS (v/v=1/199). (1) Fluorescence response of the probe (10 μM) toward glutamic acid, folate acid, lysozyme, bovine serum albumin (BSA), pepsin, ascorbic acid or glutathione in DMSO/PBS (v/v=1/199). The excitation wavelength is 430 nm. Data represent mean values±standard deviation, n=3.

FIGS. 16A-H illustrate confocal images of MDA-MB-231 cells (a-f), MCF-7 cells (g), 293T cells (h) or MDA-MB-231 cells pretreated with cRGD (e) or BSO (f) after incubation with the probe for 1 h (a), 2 h (b), 3 h (c), 4 h (d-h). The blue fluorescence from the nuclei of cells were living stained with Hoechst (Eₓ: 405 nm; Eₘ: 430-470 nm); the red fluorescence is from TPETP (Eₓ: 405 nm; Eₘ: >560 nm). All images share the same scale bar (20 μm).

FIGS. 17A-H illustrate the Real-time cell apoptosis imaging. Confocal images of MDA-MB-231 cells (a-f), MCF-7 cells (g), 293T cells (h) or MDA-MB-231 cells treated with cRGD (e) or VC (f) and incubated with the probe for 4 h with light irradiation of 1 min (a), 2 min (b), 4 min (c), 6 min (d-h). The blue fluorescence from the nuclei of cells were living stained with Hoechst (Eₓ: 405 nm; Eₘ: 430-470 nm); the green fluorescence is from the TPS (Eₓ: 405 nm; Eₘ: 505-525 nm). All images share the same scale bar (20 μm).

FIG. 18 illustrates the targeted dual-acting prodrug for real-time drug tracking and activation monitoring.

FIGS. 19A-E is an evaluation of the targeting effect of cRGD-TPE-Pt-DOX to different cells: confocal images of MDA-MB-231 (A), MCF-7 (green and red) (B) cancer cells and 293T (red) (C) normal cells after incubation with cRGD-TPE-Pt-DOX for 2 h (green and red). The masked green color represents fluorescence from cRGD-TPE-Pt-DOX (λex=488 nm) and the red color represents fluorescence from the nuclei of cells stained by DRAQ5. All images share the same scale bar (20 μm). (D) Relative fluorescence intensity of cRGD-TPE-Pt-DOX (λex=488 nm) determined in MDA-MB-231, MCF-7 and 293T cells at different incubation time. (E) Relative fluorescence intensity of cRGD-TPE-Pt-DOX determined in MDA-MB-231, MCF-7 and 293T cells with and without cRGD (50 μM) pretreatment. The error is the standard deviation from the mean (n=3, * is P<0.05).

FIG. 20 illustrates confocal images of MDA-MB-231 cells after incubation with free DOX (A, 6 h, green only), cRGD-TPE (B, 6 h, green and red), and cRGD-TPE-Pt-DOX for 1 h (C, green and red), 2 h (D, green and red), and 2 h followed by incubation in fresh medium for another 4 h (E). Blue: TPE fluorescence; green: DOX fluorescence; red: cell nuclei stained by DRAQ5. All images share the same scale bar (20 μm). (F) is a C.I. plot for cRGD-TPE-Pt-DOX demonstrating effectiveness against MDA-MB-231 cells over a wide range of drug effect levels from 75% to 25%.

FIG. 21 illustrates the synthetic route of cRGD-TPE-Pt-DOX.

FIG. 22 illustrates (A) Chemical structure of the prodrug TPECB-Pt-D5-cRGD; (B) Schematic illustration of TPECB-Pt-D5-cRGD used for cisplatin activation monitoring and image-guided combinatorial photodynamic therapy and chemotherapy for the ablation of cisplatin resistant cancer cells.

FIG. 23 illustrates (A) Photoluminescence (PL) spectra of TPECB and TPECB-Pt-D5-cRGD (10 μM) in DMSO/PBS (v/v=1/199). Inset shows the photographs taken under a hand-held 365 nm lamp. (B) Fluorescence spectra of TPECB-Pt-D5-cRGD (10 μM) incubated with GSH (100 μM) in DMSO/PBS (v/v=1/199) after different time durations. (C) Fluorescence response of TPECB-Pt-D5-cRGD (10 μM) toward 100 μM of different analysts in DMSO/PBS (v/v=1/199). (D) UV-vis absorption changes of ROS indicator 9,10-anthracenediyl-bis(methylene)dimalonic acid (ABDA) mixed with GSH-pretreated prodrug for different time duration of light irradiation. VC stands for ROS scavenger vitamin C. Data represent mean values±standard deviation, n=3.

FIG. 24 illustrates confocal images of prodrug incubated MDA-MB-231 cells (A-C, E, F), U87-MG cells (D), MCF-7 cells (G), 293T cells (H) for different time durations. For E and F, the cells were pretreated with free cRGD or buthionine sulfoximine (BSO), respectively. The red fluorescence (in FIGS. 24 B-G) is from TPETB (Ex: 405 nm; Em: >560 nm); the blue fluorescence is from cell nucleus dyed with Hoechst (Ex: 405 nm; Em: 430-470 nm). All images share the same scale bar (20 μm).

FIG. 25 illustrates the synthetic route of TPECB-Pt-D5-cRGD.

FIG. 26 is an illustration of (A) Chemical structure of the PEGylated polyprodrug PFVBT-g-PEG-DOX and (B) schematic illustration of the light regulated ROS activated on-demand drug release and the combined chemo-photodynamic therapy.

FIG. 27 is (A) Analyses of the stability and degradation of N3-PEG-TK-DOX in the presence of ROS detected at absorbance of 254 nm by HPLC. (B) Normalized UV-vis absorption spectra of DOX, TCP NPs and TCP-DOX NPs. (C) Size distribution and TEM image (inset) of TCP-DOX NPs. (D) Average hydrodynamic diameter changes of TCP-DOX NPs when incubated in water, PBS buffer or DMEM at 37° C. for 7 days (the inset digital photograph shows TCP-DOX NPs dispersed in water, PBS buffer or DMEM, indicating good dispersity). (E) Dichlorofluorescein (DCF) fluorescence intensity at 530 nm in PBS, DOX, TCPDOX NPs and TCP NPs after light irradiation for different time. VC stands for ROS scavenger vitamin C. (F) Cumulative release profiles of DOX from TCPDOX NPs without and with the light irradiation. Standard deviations are shown as error bars from three parallel experiments.

FIG. 28 is evaluation of the targeting effect of TCP-DOX NPs to different cancer cells: (A) Confocal microscopy images of MDA-MB-231 and MCF-7 cells after incubation with the NPs for 4 h. The blue fluorescence is from the nuclei of cells stained by Hoechst 33342, the red fluorescence is from PFVBT-g-PEG-DOX. All images share the same scale bar (20 μm); (B) Integrated fluorescence intensity of PFVBT-g-PEG-DOX determined in MDA-MB-231 and MCF-7 cells at different incubation time; (C) fluorescence intensity of PFVBT-g-PEG-DOX determined in MDA-MB-231 and MCF-7 cells with and without cRGD (50 μM) pretreatment. The error is the standard deviation from the mean (n=3, * is P<0.05).

FIG. 29 is detection of intracellular reactive oxygen species (ROS) production using DCF-DA staining in MDA-MB-231 cells incubated with (A) DCF-DA; (B) TCP-DOX NPs; (C) TCP-DOX NPs and DCF-DA; (D) TCP-DOX NPs and DCFDA in the presence of ROS scavenger (VC, 50 μM). Green (seen in C and D): ROS indicator DCF; Red (seen in B-D): PFVBT-g-PEG-DOX fluorescence. All images share the same scale bar (50 μm).

FIG. 30 is the synthetic scheme of PFVBT-g-PEG-DOX.

FIG. 31 illustrates the targeting effect of TCP/PTX NPs to different cancer cells: (A-B) confocal microscopy images of NPs uptake in U87-MG cells (A) with receptor overexpression and receptor negative MCF-7 cells (B), the images can be classified to blue fluorescence from the nuclei of cells dyed by Hoechst 33342, red fluorescence (seen in FIG. 31A) from TCP/PTX NPs, and the merged images of above. All images share the same scale bar (20 μm); (C) dynamic fluorescence intensity of TCP/PTX NPs determined in U87-MG and MCF-7 cells at different incubation time points; (D) confocal microscopy images of TCP/PTX NPs uptake in cRGD (50 μM) pretreated U87-MG cells and (E) mean fluorescence intensity of TCP/PTX NPs determined in U87-MG and MCF-7 cells with receptor blocking or nonblocking after 4 h incubation. The error is the standard deviation from the mean (n=3, * is P<0.05).

FIG. 32 illustrates detection of intracellular reactive oxygen production (ROS) by DCF-DA staining in U87-MG cells incubated with (A) DCF-DA with light excitation; (B) TCP/PTX NPs with light excitation (green); (C) TCP/PTX NPs and DCF-DA with light excitation (green); (D) TCP/PTX NPs and DCF-DA in the presence of ROS scavenger (vitamin C, 50 μM) with light excitation (green). E-H indicate the corresponding CP fluorescence (F-H are red). All images share the same scale bar (50 μm).

FIG. 33 illustrates the synthetic pathway to create DPBA-TPE.

FIG. 34 illustrates ROS generation of FA-AIE-TPP dots in aqueous solution at a) varied dot concentrations, and b) varied light powers upon irradiation for 300 s.

FIG. 35 illustrates CLSM images of a) MCF-7 cancer cells and b) NIH-3T3 normal cells after incubation with AIE dots and MitoTracker Green. AIE dots: Eₓ: 543 nm, Eₘ: >650 nm (red); MitoTracker Green: Eₓ=488, Eₘ=505-525 nm. c) Pearson's Coefficients between AIE dots and MitoTracker Green inside MCF-7 and NIH-3T3 cells. The scale bar size is 10 μm for all images.

FIG. 36 illustrates viabilities of MCF-7 cancer cells and NIH-3T3 normal cells after incubation with a) AIE-TPP, b) AIE-FA, c) FA-AIE-TPP dots at varied concentrations, followed by white light irradiation. d) and e) Annexin V labeled MCF-7 cells after incubation with FA-AIE-TPP dots without (d) or with (e) light irradiations (green). d) and e) share the same scale bar.

FIG. 37 illustrates mitochondria potential changes of FA-AIE-TPP dots treated MCF-7 cancer cells measured by JC 1 after light irradiation for a) 0, b) 5, and c) 10 min. All the images share the same scale bar. The JC Monomor fluoresces green, the JC Aggregate fluoresces red, the JC Merge fluoresces as follows: (A) is red-orange and green, (B) is green, and (C) is green.

FIG. 38 illustrates a) White field image of FA-AIE-TPP dots treated NIH-3T3 and MCF-7 Cells before (up) and after 72 h culture (bottom). Cells were incubated with FA-AIE-TPP dots (20 μg/mL based on DPBA-TPE mass concentration) for 4 h, followed by light exposure (100 mW/cm²) for 10 min. b) The effects of AIE dots treatment on migration of MCF-7 cells with and without light irradiation.

### DETAILED DESCRIPTION OF THE INVENTION

A description of example embodiments of the invention follows.

### DEFINITIONS

All definitions of substituents set forth below are further applicable to the use of the term in conjunction with another substituent.

“Alkyl” means a saturated aliphatic branched or straight-chain monovalent hydrocarbon radicals, typically C₁-C₁₀, preferably C₁-C₆. “(C₁-C₆) alkyl” means a radical having from 1-6 carbon atoms in a linear or branched arrangement. “(C₁-C₆)alkyl” includes methyl, ethyl, propyl, butyl, tert-butyl, pentyl and hexyl.

“Alkylene” means a saturated aliphatic straight-chain divalent hydrocarbon radical. Thus, “(C₁-C₆)alkylene” means a divalent saturated aliphatic radical having from 1-6 carbon atoms in a linear arrangement. “(C₁-C₆)alkylene” includes methylene, ethylene, propylene, butylene, pentylene and hexylene.

“Cycloalkyl” means saturated aliphatic cyclic hydrocarbon ring. Thus, “C₃-C₈ cycloalkyl” means (3-8 membered) saturated aliphatic cyclic hydrocarbon ring. C₃-C₈ cycloalkyl includes, but is not limited to cyclopropyl, cyclobutyl, cyclopentyl, cyclohexyl, cycloheptyl, and cyclooctyl. Preferably, cycloalkyl is C₃-C₆ cycloalkyl.

The term “alkoxy” means —O-alkyl; “hydroxyalkyl” means alkyl substituted with hydroxy; “aralkyl” means alkyl substituted with an aryl group; “alkoxyalkyl” mean alkyl substituted with an alkoxy group; “alkylamine” means amine substituted with an alkyl group; “cycloalkylalkyl” means alkyl substituted with cycloalkyl; “dialkylamine” means amine substituted with two alkyl groups; “alkylcarbonyl” means —C(O)-A*, wherein A* is alkyl; “alkoxycarbonyl” means —C(O)-OA*, wherein A* is alkyl; and where alkyl is as defined above. Alkoxy is preferably O(C₁-C₆)alkyl and includes methoxy, ethoxy, propoxy, butoxy, pentoxy and hexoxy.

“Cycloalkoxy” means a —O-cycloalkyl, wherein the cycloalkyl is as defined above. Exemplary (C₃-C₇)cycloalkyloxy groups include cyclopropoxy, cyclobutoxy, cyclopentoxy, cyclohexoxy and cycloheptoxy.

The term “aryl” used alone or as part of a larger moiety as in “arylalkyl”, “arylalkoxy”, “aryloxy”, or “aryloxyalkyl”, means carbocyclic aromatic rings. The term “carbocyclic aromatic group” may be used interchangeably with the terms “aryl”, “aryl ring” “carbocyclic aromatic ring”, “aryl group” and “carbocyclic aromatic group”. An aromatic ring typically has 6-16 ring atoms. A “substituted aryl group” is substituted at any one or more substitutable ring atom. The term “C₆-C₁₆ aryl” as used herein means a monocyclic, bicyclic or tricyclic carbocyclic ring system containing from 6 to 16 carbon atoms and includes phenyl (Ph), naphthyl, anthracenyl, 1,2-dihydronaphthyl, 1,2,3,4-tetrahydronaphthyl, fluorenyl, indanyl, indenyl and the like. In particular embodiments, the aryl group is (C₆-C₁₀)aryl. The (C₆-C₁₀)aryl(C₁-C₆)alkyl group connects to the rest of the molecule through the (C₁-C₆)alkyl portion of the (C₆-C₁₀)aryl(C₁-C₆)alkyl group. An aromatic ring includes monocyclic and polycyclic rings.

“Hetero” refers to the replacement of at least one carbon atom member in a ring system with at least one heteroatom selected from N, S, and O. The heteroatom can optionally carry a charge. When N is the heteroatom of a ring system, it may be additionally substituted by one or more substituents including H, OH, O⁻, alkyl, aryl, heterocyclyl, cycloalkyl or alkenylene, wherein any of the alkyl, aryl, heterocyclyl, cycloalkyl or alkenylene may be optionally and independently substituted by one or more substituents selected from halo, cyano, nitro, hydroxyl, phosphate (PO₄³⁻) or a sulfonate (SO₃⁻).

“Heterocycle” means a saturated or partially unsaturated (3-7 membered) monocyclic heterocyclic ring containing one nitrogen atom and optionally 1 additional heteroatom independently selected from N, O or S. When one heteroatom is S, it can be optionally mono- or di-oxygenated (i.e., —S(O)— or —S(O)₂—). Examples of monocyclic heterocycle include, but not limited to, azetidine, pyrrolidine, piperidine, piperazine, hexahydropyrimidine, tetrahydrofuran, tetrahydropyran, morpholine, thiomorpholine, thiomorpholine 1,1-dioxide, tetrahydro-2H-1,2-thiazine, tetrahydro-2H-1,2-thiazine 1,1-dioxide, isothiazolidine, or isothiazolidine 1,1-dioxide. The heterocycle can be optionally fused to a carbocyclic ring, as in, for example, indole.

The term “heteroaryl”, “heteroaromatic”, “heteroaryl ring”, “heteroaryl group” and “heteroaromatic group”, used alone or as part of a larger moiety as in “heteroarylalkyl” or “heteroarylalkoxy”, refers to aromatic ring groups having five to fourteen total ring atoms selected from carbon and at least one (typically 1-4, more typically 1 or 2) heteroatoms (e.g., oxygen, nitrogen or sulfur). They include monocyclic rings and polycyclic rings in which a monocyclic heteroaromatic ring is fused to one or more other carbocyclic aromatic or heteroaromatic rings. Typically a heteroaromatic ring comprises 5-14 total ring atoms. The term “5-14 membered heteroaryl” as used herein means a monocyclic, bicyclic or tricyclic ring system containing one or two aromatic rings and from 5 to 14 total atoms of which, unless otherwise specified, one, two, three, four or five are heteroatoms independently selected from N, NH, N(C₁₋₆alkyl), O and S. (C₃-C₁₀)heteroaryl includes furyl, thiophenyl, pyridinyl, pyrrolyl, imidazolyl, and in preferred embodiments of the invention, heteroaryl is (C₃-C₁₀)heteroaryl.

“Halogen” and “halo” are interchangeably used herein and each refers to fluorine, chlorine, bromine, or iodine.

“Cyano” means —C≡N.

“Nitro” means —NO₂.

As used herein, an amino group may be a primary (—NH₂), secondary (—NHRₓ), or tertiary (—NRₓR_{y}), wherein Rₓ and R_{y} may be any alkyl, aryl, heterocyclyl, cycloalkyl or alkenylene, each optionally and independently substituted with one or more substituents described above. The Rₓ and R_{y} substituents may be taken together to form a “ring”, wherein the “ring”, as used herein, is cyclic amino groups such as piperidine and pyrrolidine, and may include heteroatoms such as in morpholine.

The terms “haloalkyl”, “halocycloalkyl” and “haloalkoxy” mean alkyl, cycloalkyl, or alkoxy, as the case may be, substituted with one or more halogen atoms. The term “halogen” means F, Cl, Br or I.

The term “acyl group” means —C(O)A*, wherein A* is an optionally substituted alkyl group or aryl group (e.g.; optionally substituted phenyl).

An “alkylene group” is represented by —[CH₂]_{z}—, wherein z is a positive integer, preferably from one to eight, more preferably from one to four.

An “alkenylene group” is an alkylene in which at least a pair of adjacent methylenes are replaced with —CH═CH—.

The term benzyl (Bn) refers to —CH₂Ph.

The term “Alkenyl” means a straight or branched hydrocarbon radical including at least one double bond. The (C₆-C₁₀)aryl(C₂-C₆)alkenyl group connects to the remainder of the molecule through the (C₂-C₆)alkenyl portion of (C₆-C₁₀)aryl(C₂-C₆)alkenyl.

A “conjugated system” as used herein, is a system of connected atoms having p-orbitals with delocalized electrons. Such a system generally alternates single and multiple (e.g., double) bonds, and in certain embodiments also contains atoms having a lone pair, radical atoms, or carbenium ions. Conjugated systems can be cyclic or acyclic. Naphthalene is an example of a conjugated system.

Pharmaceutically acceptable salts of the compounds of the present invention are also included. For example, an acid salt of a compound of the present invention containing an amine or other basic group can be obtained by reacting the compound with a suitable organic or inorganic acid, resulting in pharmaceutically acceptable anionic salt forms. Examples of anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, and triethiodide salts.

Salts of the compounds of the present invention containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine.

“Aggregation-induced emission” refers to a property in which a fluorophore, when dispersed, for example in organic solvent, emits little or no light. Upon aggregation of fluorophore molecules, however, for example in the solid state or in water due to the hydrophobicity of the fluorophore, light emission from the fluorophore is significantly enhanced.

A “biocompatible matrix”, as used herein, is a scaffold that supports a chemical compound or a polymer that serves to perform an appropriate function in a specific application without causing an inappropriate or undesirable effect in a host system. Examples of biocompatible matrices include poly(ethylene glycol), 1,2-Distearoyl-sn-glycero-3-phosphoethanolamine-N-[methoxy(polyethylene glycol)] (DSPE-PEG), poly(DL-lactide-co-glycolide), chitosan, bovine serum albumin and gelatin. In certain embodiments, the polyethylene glycol comprises from about 5 to about 115 monomeric units. In other embodiments, the polyethylene glycol comprises from about 6 to about 113 monomeric units.

A “lipid”, as used herein, means hydrophobic or amphiphilic small molecules. In certain embodiments, lipids include sterols, fatty cadids, glycerides, diglycerides, triglycerides, certain fat-soluble vitamins and phospholipids.

A “target recognition motif” as used herein, is a chemical moiety having an affinity for a biological target such as a protein, a peptide, or a receptor in the cell membrane. A target recognition motif can comprise a peptide, a protein, an oligonucleotide, or an organic functional group having an affinity for a specific target structure.

A “linking moiety” as used herein, is a chemical moiety that links two or more groups through one covalent bond or through a series of covalent bonds. Example linking moieties include disulfide groups, amino groups, 2-nitrobenzyl derivatives, sulfones, hydrazones, vicinal diols, or simply one or more covalent bonds. Further examples of linking moieties may be found in Table 1 of Bioorg. Med. Chem., 2012, 20, 571-582, the contents of which are incorporated herein by reference. The covalent bonds in the linking moiety sever upon exposure to an external stimulus. Examples of external stimuli include, but are not limited to, exposure to a chemical compound, exposure to a reactive oxygen species, exposure to a specific wavelength of light, exposure to a specific pH, exposure to a specific force.

A “chemical responsive” linking moiety is a linking moiety which includes a covalent bond capable of breaking upon exposure to a specific chemical composition. An example of a chemical responsive linking moiety is disulfide (—S—S—).

A “reactive oxygen species (ROS)” linking moiety is a moiety which can be cleaved upon exposure to a reactive oxygen species. Examples of ROS linking moieties include:

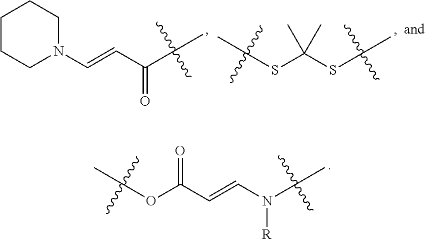

A “pH responsive” linking moiety is a moiety which can be cleaved upon exposure to a specific pH or pH range. An example of a pH responsive linking moiety includes:

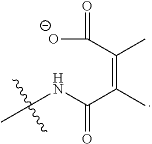 [SMILES: *NC(=O)/C(C)=C(/C)C(=O)[O-]]

A “light responsive” linking moiety is a moiety which can be cleaved upon exposure to a specific wavelength or a range of wavelengths of light. Examples of light responsive linking moieties include:

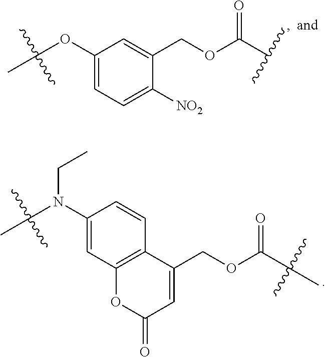 [SMILES: *Oc1ccc([N+](=O)[O-])c(COC(*)=O)c1 ; *C(=O)OCc1cc(=O)oc2cc(N(*)CC)ccc12]

As used herein, “spectroscopy” encompasses any method by which matter reacts with radiated energy. This includes, but is in no way limited to, microscopy, fluorescence microscopy, UV/Vis spectrometry, and flow cytometry. A “microplate reader” as used herein, means a laboratory instrument that measures, for example, fluorescence, absorbance and luminescence of samples contained in a microplate.

Chemotherapeutic drugs include cytotoxic anti-neoplastic compounds and compositions. Example chemotherapeutic drugs include doxorubicin, paclitaxel, melphalan, camptothecin, and gemcitabine.

A “prodrug” as used herein, is a therapeutic compound that is typically administered to a subject in its inactive form and is converted to its active form in the body of the subject. For example, a prodrug may include a platinum (IV) [Pt (IV)] complex that is converted to an active platinum (II) [Pt (II)] complex. In an example embodiment, the Pt(II) complex is cisplatin, and the precursor Pt(IV) complex is an octahedral complex, wherein the xy plane includes chloro and amino ligands, and the complex further includes two additional axial ester ligands. In certain embodiments, such a conversion occurs via reduction with a chemical reagent. In certain other embodiments, such a conversion occurs via metabolic processes.

Tetraphenylethylene, or TPE, is:

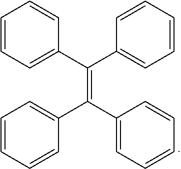 [SMILES: c1ccc(C(=C(c2ccccc2)c2ccccc2)c2ccccc2)cc1]

A “biological sample”, as used herein, includes cellular extracts, live cells, and tissue sections. A cellular extract is lysed cells from which insoluble matter has been removed via centrifugation. A “live cell” is a living cell culture for in vitro analysis. A live cell can refer to a single cell or a plurality of cells. A “tissue section” is a portion of tissue suitable for analysis. A tissue section can refer to a single tissue section or a plurality of tissue sections.

As used herein, “spectroscopy” encompasses any method by which matter reacts with radiated energy. This includes, but is in no way limited to, microscopy, fluorescence microscopy, UV/Vis spectrometry, and flow cytometry.

A “change in fluorescence signal” as used herein, can be used to indicate a change in the fluorescence intensity of a sample after incubation with a biological sample, as compared to a baseline exposure. In some embodiments of the invention, the change in fluorescence intensity is an increase in fluorescence intensity. Alternately, a change in fluorescence can be a change in the color of the fluorescence. A change in the color of the fluorescence can be a change in the color hue of the fluorescence (e.g a green hue versus a red hue), or can be a change in the tint or saturation of the fluorescence (e.g. a light red versus a dark red).

As used herein, the term “incubation” or alternately, “incubating” a sample means mixing a sample. Alternately, incubating means mixing and heating a sample. “Mixing” can comprise mixing by diffusion, or alternately by agitation of a sample.

In certain embodiments, live cells are the target of a treatment or therapeutic regimen. In some embodiments, live cells can be cancer cells that are the therapeutic target of a prodrug.

A “nanoparticle” as used herein, is a small object that behaves as a single unit with respect to its transport and properties. In certain embodiments, a nanoparticle ranges in size from 5 nm to 5000 nm. In certain embodiments of the invention, the conjugated polymers described herein self-assemble in solution to form nanoparticles.

[symbol] represents a point of attachment between two atoms.

“Agent,” as used herein, refers to a chemical or biological material that can be used in a therapeutic regiment. Example agents include DNA, RNA, SiRNA, pharmaceuticals, or drugs.

### Example 1

### Aggregation-Induced Emission Fluorogens for Cell Tracing

Fabrication of surface functionalized green emissive AIE dots for longterm cell tracing using an AIE fluorogen 4,7-bis[4-(1,2,2-triphenylvinyl)phenyl]benzo-2,1,3-thiadiazole (BTPEBT) as an example is reported. BTPEBT is an example of a conjugated system that can be used in the present invention. A mixture of lipid-poly(ethylene glycol) (PEG) and lipid-PEG-maleimide was chosen as the encapsulation matrix to endow BTPEBT into AIE dots with biocompatibility and surface functionality. A cell penetrating peptide derived from HIV-1 transactivator of transcription protein (Tat) was further conjugated to the dot surface to yield AIE-Tat dots with high cellular internalization efficiency. The AIE-Tat dots showed an emission maximum at 547 nm, similar to GFP, with a high quantum yield of 63%, and stable green fluorescence in either different pH conditions or long time incubation in buffer solution for over 10 days. The cell labelling performances of the AIE-Tat dots in the in vitro studies were compared to the classical calcium phosphate mediated GFP transfection method under similar experimental conditions. It was found that the AIE-Tat dots have the capability to label all the tested human cells with high brightness and ˜100% labelling efficiency; significantly outperforming the GFP plasmid transfection approach which only showed varied and relatively low GFP labelling efficiency. Moreover, in the cell tracing experiment, AIE-Tat dots are able to trace the activity of HEK293T cells for over 10 days, while pMAX-GFP can only trace the same cell population for a maximum of 3 days.

BTPEBPT is represented by the following structural formula:

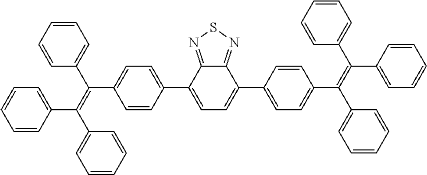 [SMILES: c1ccc(C(=C(c2ccccc2)c2ccc(-c3ccc(-c4ccc(C(=C(c5ccccc5)c5ccccc5)c5ccccc5)cc4)c4nsnc34)cc2)c2ccccc2)cc1]

Fabrication and characterization of AIE-Tat Dots.

The selected AIE fluorogen, 4,7-bis[4-(1,2,2-triphenylvinyl)phenyl]benzo-2,1,3-thiadiazole (BTPEBT) was synthesized via Suzuki coupling reaction and its structure was confirmed by 1H and 13C NMR. The AIE effect of BTPEBT was studied by measuring its photoluminescence (PL) spectra in tetrahydrofuran (THF)/water mixture with different water fraction (fw). Along with increasing of fw, BTPEBT initially showed gradually quenched fluorescence, followed by fluorescence recovery.

To fabricate the ultra-bright and biocompatible BTPEBT-loaded AIE dots, 1,2-distearoyl-snglycero-3-phosphoethanolamine-N-[methoxyl-(polyethylene glycol)-2000] (DSPE-PEG2000) and its maleimide group ended derivative, 1,2-distearoyl-sn-glycero-3-phosphoethanolamine-N-[maleimide (polyethylene glycol)-2000] (DSPE-PEG2000-Mal), were used as the encapsulation matrices to embed BTPEBT49, 50, 58. The AIE dots are formed through self-assemble driven by hydrophobicity changes of the solvent. The presence of PEG shells helps provide functional groups for further chemical or biological conjugation, and minimize nonspecific interaction with biological species. After THF evaporation, a cell membrane penetration peptide (Arg-Lys-Lys-Arg-Arg-Gln-Arg-Arg-Arg-Cys) (SEQ ID NO: 1) derived from HIV-1 transactivator (Tat) of transcription protein was conjugated onto AIE dot through click reaction between surface maleimide and the thiol groups at the C-terminus of the peptide. The yielded AIE-Tat dots were further filtered using a 0.2 μm syringe filter and stored at 4° C.

The AIE-Tat dots have two absorption peaks centred at 318 and 422 nm, with a molar extinction coefficient of 5.9 107 M-1 cm-1 at 422 nm on the basis of dot concentration. The AIE-Tat dots show an emission maximum at 547 nm, with a large Stokes shift of 125 nm and a high fluorescence quantum yield of 63, measured using Rhodamine 6G in methanol as the standard (quantum yield=93%).

### In Vitro Cellular Imaging by AIE-Tat Dots.

The in vitro cellular imaging performance of AIE-Tat dots was evaluated using human embryonic kidney 293T (HEK293T) cells as a model. AIE-Tat dots are passively loaded into adherent HEK293T cells by incubating them with AIE-Tat dots at different concentrations (0 to 2 nM). After 2 h incubation, the fluorescence images of HEK293T cells were examined using confocal laser scanning microscopy (CLSM) with emission signal collected above 505 nm upon excitation at 488 nm. A progressive increase in green fluorescent signal from the cellular membrane to cytoplasm was observed along with increase in AIE-Tat dots loading concentrations. At low AIE-Tat dot loading concentration of 50 pM or lower, the AIE-Tat dots tend to bind to cell membrane surface, whereas negligible fluorescence was detected from the cytoplasm. However, at a high incubation concentration of 2 nM, the accumulation of green fluorescence in the cytoplasm is clearly observed.

### In Vitro Cell Labelling Comparison Between GFP and AIE-Tat Dots.

Next, the application of AIE-Tat dots as a generic labelling agent was further examined using a panel of human cells of different tissue origin. HEK293T cells, human colon adenocarcinoma SW480 cells (SW480), human colon adenocarcinoma DLD-1 cells (DLD-1), normal human colon mucosal epithelial cells (NCM460 cells), normal human primary dermal fibroblast cells (NHDF cells), and human bone marrow derived stem cells (BMSCs) were chosen as in vitro model cell lines. Calcium phosphate transfection method was employed as a standard benchmark to transfect these cells to express GFP61. pMAX-GFP plasmid (5 μg/well) that drives the GFP expression from copepod Pontellina p. was incubated with the cells overnight. Similar procedures were also repeated for cells to be labeled by AIE-Tat dots (2 nM). The labeling efficiencies by GFP or AIE-Tat dots are assessed by means of flow cytometry analysis. Among the cells tested, only HEK293T cells display high GFP expression of 70%, while only 0 to 30% of SW480, DLD-1, NCM460, NHDF and BMSCs are GFP-positive with extremely low mean fluorescence, which falls just above the critical points that was considered as cell auto-fluorescence. These results are similar to literature reports, where nonviral transfection method present relative low and cell type dependent transfection. On the contrary, AIE-Tat dots showed nearly 100% labeling efficiencies towards all these tested cell lines, with over 100-fold higher mean fluorescence intensity as compared to GFP labeled cells. This result clearly indicates the superior cell labeling ability of AIE-Tat dots over GFP. Similar phenomena are also confirmed by CLSM images, where the GFP-positive cells among these different cell lines varied in a large range, further indicating the limitation of GFP transfection method in practical applications. On the other hand, all the cells treated with AIE-Tat dots showed bright green fluorescence, despite of cell types. In addition, AIE-Tat dots showed higher photostability inside the cells, where the signal loss of AIE-Tat dotsstaining cells is less than 15%, while GFP transfected cells lost 40% of their fluorescence after 10 min of continuous laser scanning. It is noteworthy that the cells directly labeled by AIE-Tat dots can be immediately detected by CLSM and flow cytometry, while a lag period of several to 24 hours between plasmid introduction and GFP expression exists for GFP transfection. Collectively, our results suggest that AIE-Tat dots outperform the traditional fluorescent protein-based live cell labelling on several fronts, thus making them a promising choice for cell imaging and tracing.

### Synthesis of AIE-Tat Dots.

A THF solution (1 mL) containing BTPEBT (0.5 mg) and DSPE-PEG2000 (0.5 mg) and DSPE-PEG2000-Mal (0.5 mg) was poured into water (10 mL) under sonication using a microtip probe sonicator at 12 W output (XL2000, Misonix Incorporated, NY). The mixture was further placed in dark in fume hood for THF evaporation at 600 rpm overnight. The AIE dots (1.8 mL) were further mixed and reacted with HIV1-Tat peptide (3×105 M). After reaction for 4 h at room temperature, the solution was dialysed against MilliQ water for 2 days to eliminate the excess peptide. The AIE dot suspension was further purified by filtering through a 0.2 μm syringe driven filter. The Tat-AIE dots were collected for further use.

### Cell Labeling by AIE-Tat Dots.

Human embryonic kidney HEK293T cells were cultured in chamber (LAB-TEK, Chambered Coverglass System) at 37° C. After 80% confluence, the medium was removed; the adherent cells were washed twice with 1 PBS buffer. AIE-Tat dots with different concentrations (1 pM, 5 pM, 10 pM, 200 pM, 1 nM, and 2 nM suspended in cell culture medium were then added into the chamber. After 2 h incubation, the cells were washed twice with 1 PBS buffer. After washing twice with 1 PBS buffer, the cells were immediately imaged by confocal laser scanning microscope (CLSM). For comparison with GFP transfection method, SW480, DLD-1, NCM460, normal human primary dermal fibroblast (NHDF) cells, and HEK293T cells were cultured in 6-well plate. After 80% confluence, the adherent cells were washed twice with 1 PBS, AIE-Tat dots (2 nM) suspended in cell culture media were then added into each well. After overnight incubation, the cells were washed twice with 1 PBS buffer, trypsinalized and then analyzed by flow cytometry measurements using Cyan-LX (DakoCytomation) and the histogram of each sample was obtained by counting 10,000 events.

### Cytotoxicity of AIE-Tat Dots.

The metabolic activity of HEK293T cells was evaluated using methylthiazolyldiphenyltetrazolium bromide (MTT) assays. HEK293T cells were seeded in 96-well plates (Costar, IL, USA) at a density of 4×104 cells/mL, respectively. After 24 h incubation, the old medium was replaced by AIE-Tat dots suspension at concentrations of 2, 5, and 10 nM, and the cells were then incubated for 24 h and 48 h, respectively. The wells were then washed with 1×PBS buffer and 100 μL of freshly prepared MTT (0.5 mg/mL) solution in culture medium was added into each well. The MTT medium solution was carefully removed after 3 h incubation. Filtered DMSO (100 μL) was then added into each well and the plate was gently shaken for 10 min at room temperature to dissolve all the precipitates formed. The absorbance of MTT at 570 nm was monitored by a microplate reader (Genios Tecan). Cell viability was expressed by the ratio of the absorbance of the cells incubated with AIE-Tat dots to that of the cells incubated with culture medium only.

### Tetraphenylethene AIE Fluorogens

Propeller-shaped fluorogens that show AIE, such as tetraphenylethene (TPE) are non-emissive in the molecularly dissolved state, but are induced to emit strong fluorescence in the aggregation state. Similarly, they can be used for image-guided photodynamic therapy.

PDT represents a well-consolidated but gradually expanding approach to the treatment of cancer. It involves excitation of photosensitizers with specific light wavelengths, which is followed by intersystem crossing (ISC) from its lowest singlet excited state (S₁) to lowest triplet excited state (T₁); subsequently, energy transfer from the T₁ of PSs to ground-state oxygen (³O₂) generates the ROS (Scheme 1), which causes oxidative damage of targets.

The primary cytotoxic agent involved in this photodynamic process is singlet oxygen, the efficient generation of which is relative habitually to the ISC efficiency of the sensitizer and concentration quenching of excited state.

To improve the ISC efficiency, many recently reported photosensitizers incorporate heavy atoms into their structures to enhance the spin-orbit perturbations. However, incorporation of heavy atoms such as selenium, iodine, bromine, and certain lanthanides has generally been reported to cause increased “dark toxicity”. It is thus important to propose alternative approaches to achieve strong ISC without using heavy atoms to minimize dark toxicity. Previous studies have shown that the ISC rate constants could be estimated from equation 1. Herein, H_{SO} is the Hamiltonian for the spin-orbit perturbations (SOP) and ΔE_{S1-T1} (ΔE_{ST}) is the energy gap between S₁ and T₁ states. ISC can be modeled by mixing of T₁ with S₁ states due to SOP. This equation shows that the efficiency of ISC can be enhanced by reducing ΔE_{ST} at a similar level of SOP.

$${k}_{ISC} ∝ \frac{{〈 {T}_{1} \vert {H}_{SO} \vert {S}_{1} 〉}^{2}}{( Δ {E}_{{S}_{1} - {T}_{1}} )} \qquad ( 1 )$$

Concentration quenching of excited state is another common problem with conventional photosensitizers (PSs), especially the widely used porphyrin derivatives, which tend to aggregate via π-π stacking due to their rigid planar structures and hydrophobic nature, resulting in aggregation-caused quenching (ACQ) and remarkable reduction in ROS generation efficiency. The quenching is more severe when the PSs are encapsulated into nanocarriers, which leads to significant decrease of their fluorescence and photodynamic efficiency.

Efficiency of the AIEgen photosensitizer can be increased by manipulating the HOMO-LUMO distribution by incorporation of electron donor and acceptor into π conjugated systems to control the ΔE_{ST} values. Accordingly, in another example embodiment of the present invention, a series of AIE-active materials incorporated with dicyanovinyl and methoxy as the electron acceptor and donor with similar molecular structures were synthesized and purified with high yields. Their ΔE_{ST} values were controlled by HOMO-LUMO engineering, resulting in coherent modulation of their ability to generate singlet oxygen. The work demonstrated for the first time a practical example of theory-guided excited state design to achieve efficient cytotoxic singlet oxygen generation for photodynamic therapy.

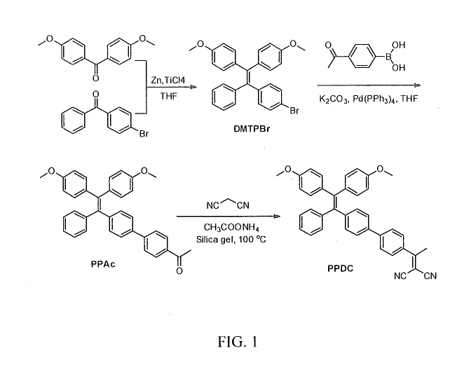

The molecular design is based on the following considerations: (1) tetraphenylethylene (TPE) is AIE-active, and the AIE characteristics can be retained after chemical modification; (2) small ΔE_{ST} values can be achieved by intramolecular charge transfer within molecular systems containing spatially separated donor and acceptor moieties; (3) benzene is often used as a π bridge for HOMO-LUMO engineering; (4) similar molecular structures will lead to similar level of SOP, so the relationship between ΔE_{ST} and ROS generation can be better understood. Accordingly, based on the parent TPE, a series of AIE-active materials, TPDC, TPPDC and PPDC, incorporated with dicyanovinyl and methoxy as the electron acceptor and donor with similar molecular structures were synthesized and purified with high yields. The molecular structures, HOMO and LUMO distribution and ΔE_{ST} values of all three compounds are shown in FIG. 1. As predicted by time-dependent DFT (TD-DFT), the ΔE_{ST} of TPDC, TPPDC and PPDC are 0.48, 0.35 and 0.27 eV, respectively. As compared to most dyes that are reported to have ΔE_{ST}≧1 eV^{[13]}, these AIE fluorogens exhibited relatively small ΔE_{ST}, suggesting a potentially high ISC rate and thus possibly efficient ROS generation.

Examples of these AIE photosensitizers include:

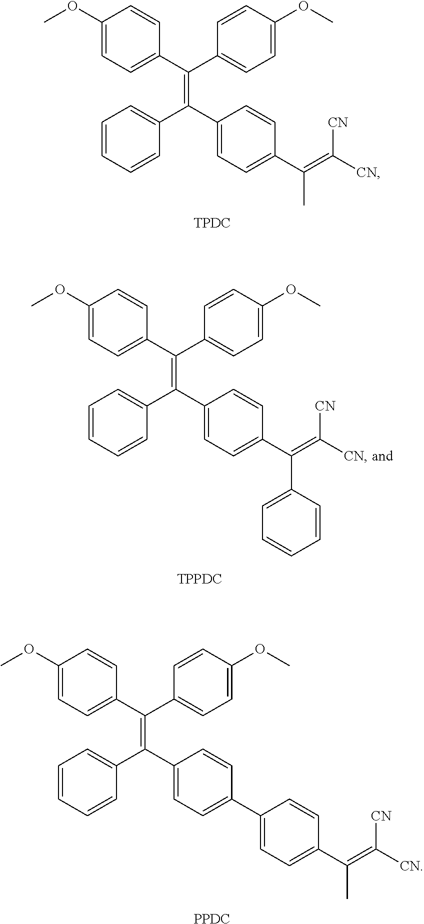

A synthetic route for PPDC is described in FIG. 1. Synthetic routes for TPDC and TPPDC are described in Y. Yuan, C. J. Zhang, M. Gao, R. Zhang, B. Z. Tang, B. Liu, Angew. Chem. Int. Ed, 54(6): 1780-86 (2015); and F. Hu, Y. Huang, G. Zhang, R. Zhao, H. Yang, D. Zhang, Anal. Chem. 2014, 86, 7987-7995.

Similar to the conjugated system described above, these AIE fluorogens can be encapsulated for delivery by, for example, 1,2-Distearoyl-sn-glycero-3-phosphoethanolamine-N-maleimide(polyethyleneglycol)-3000] (DSPE-PEG₃₀₀₀-Mal), as described above.

Additional examples of AIE fluorogens useful in the present invention further include:

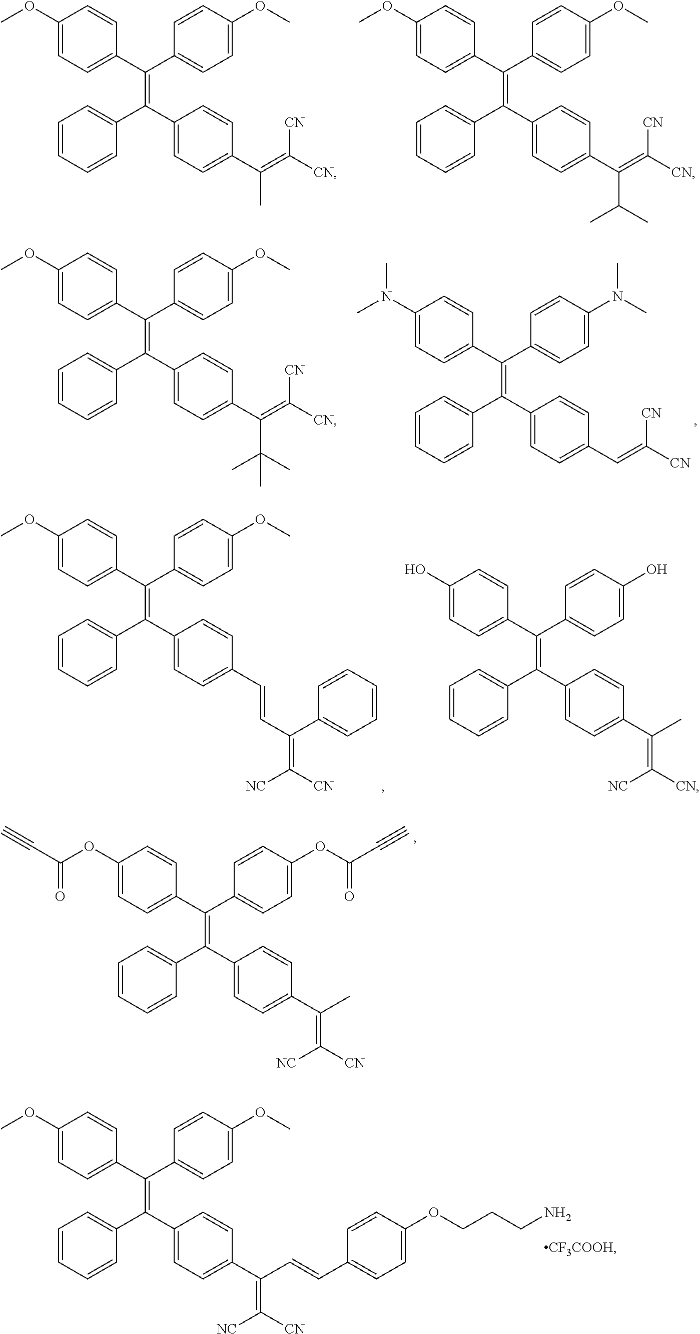

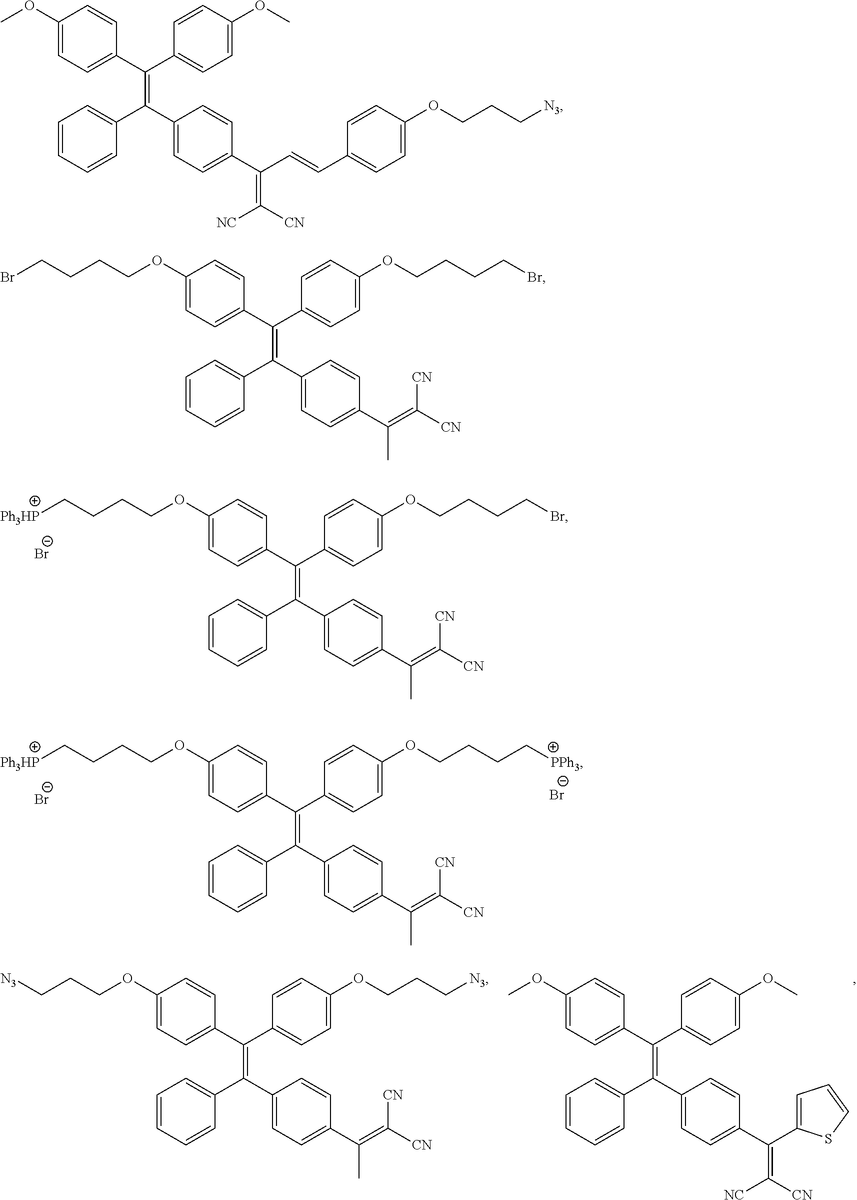

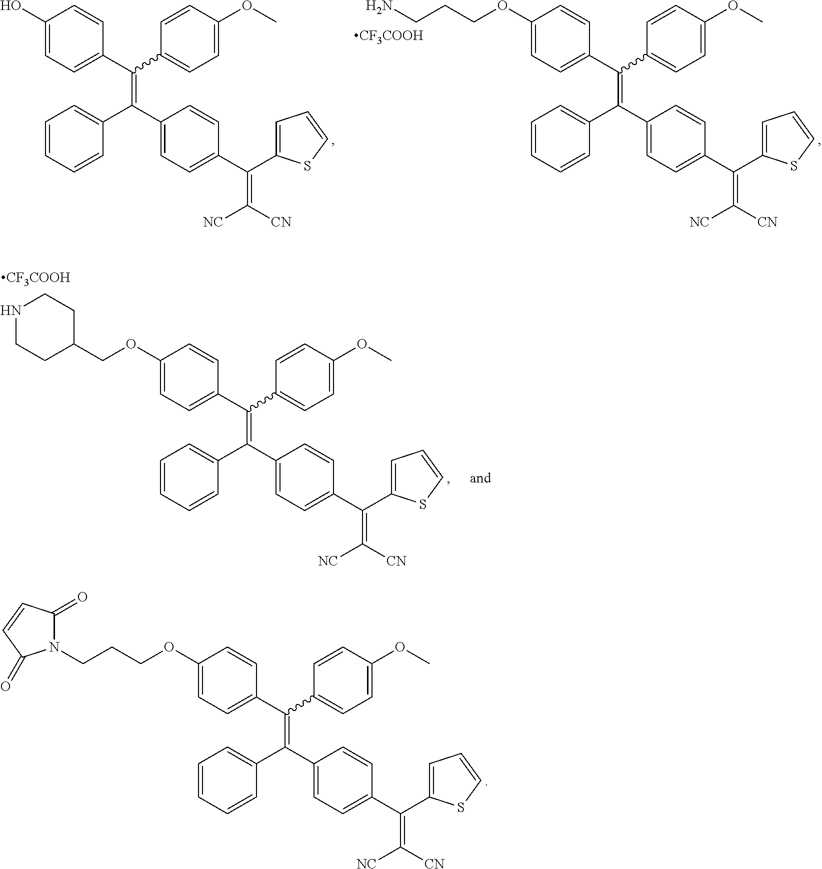 [SMILES: COc1ccc(C(=C(c2ccccc2)c2ccc(C(=C(C#N)C#N)c3cccs3)cc2)c2ccc(O)cc2)cc1 ; "·CF3COOH" ; COc1ccc(C(=C(c2ccccc2)c2ccc(C(=C(C#N)C#N)c3cccs3)cc2)c2ccc(OCCCN)cc2)cc1 ; "·CF3COOH" ; COc1ccc(C(=C(c2ccccc2)c2ccc(C(=C(C#N)C#N)c3cccs3)cc2)c2ccc(OCC3CCNCC3)cc2)cc1 ; COc1ccc(C(=C(c2ccccc2)c2ccc(C(=C(C#N)C#N)c3cccs3)cc2)c2ccc(OCCCN3C(=O)C=CC3=O)cc2)cc1]

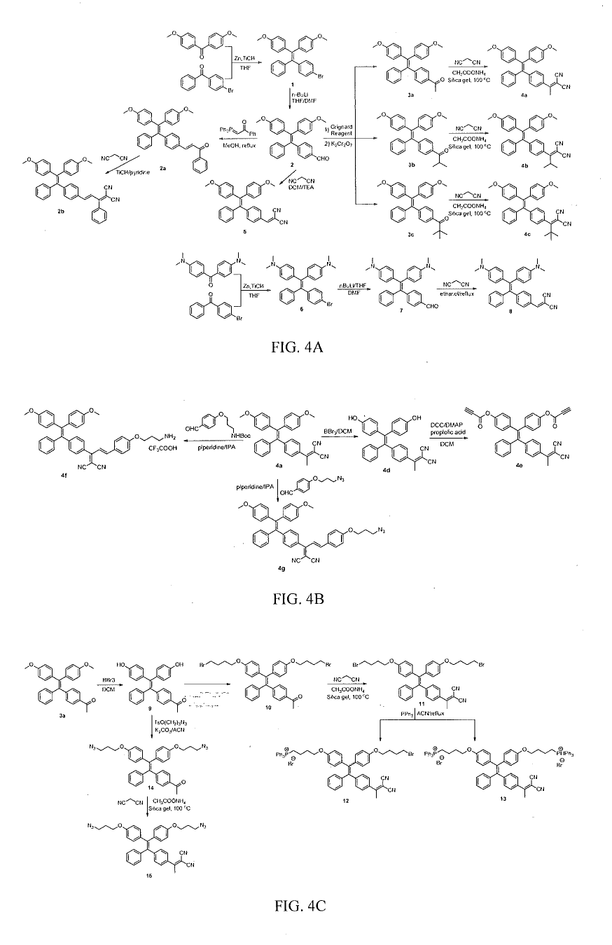

Synthetic schemes for the structures described above can be found in FIGS. 4A-C. The methods for synthesis are described below, compound number corresponds to the number in FIGS. 4A-C:

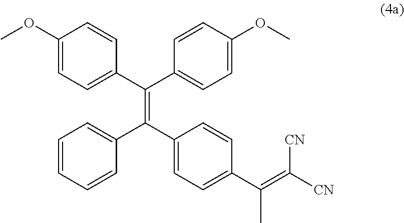 [SMILES: COc1ccc(C(=C(c2ccccc2)c2ccc(C(C)=C(C#N)C#N)cc2)c2ccc(OC)cc2)cc1]

Compound 3a (25 mg, 0.06 mmol), malononitrile (30 mg, 0.40 mmol) and ammonium acetate (43 mg, 0.56 mmol) were dissolved in the mixture of dichloromethane (5 ml) and methanol (1 ml). Then silica gel (580 mg) was added to the above mixture. Then the solvent was removed under reduced pressure. The resulting mixture was heated at 100° C. for 4 hours. The mixture was cooled down and subsequently separated with chromatography (hexane/ethyl acetate=20/1) to give the desired product (15 mg, 53.6%), ¹H NMR (400 MHz, CDCl₃) δ 7.34 (d, J=8.0 Hz, 2H), 7.13 (m, 5H), 7.02 (d, J=6.0 Hz, 2H), 6.93 (m, 4H), 6.67 (t, J=8.8 Hz, 4H), 3.75 (s, 3H), 3.74 (s, 3H), 2.57 (s, 3H); ¹³C NMR (100 MHz, CDCl₃) δ 174.4, 158.6, 158.4, 149.3, 143.3, 142.5, 137.5, 135.5, 135.4, 132.6, 132.5, 131.9, 131.3, 128.0, 126.9, 126.5, 113.3, 113.0, 55.1, 23.8; MS (ESI) calcd for [M−H]⁻: 481.19, found: 481.30.

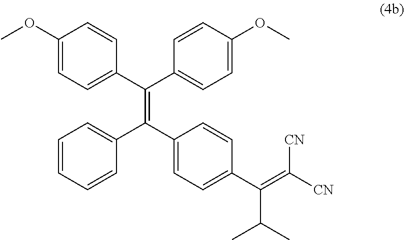 [SMILES: COc1ccc(C(=C(c2ccccc2)c2ccc(C(=C(C#N)C#N)C(C)C)cc2)c2ccc(OC)cc2)cc1]

Compound 3b (27 mg, 0.07 mmol), malononitrile (25 mg, 0.38 mmol) and ammonium acetate (30 mg, 0.38 mmol) were dissolved in a mixture of dichloromethane (5 mL) and methanol (1 mL). Silica gel (505 mg) was then added to the above mixture, and the solvent was removed under reduced pressure. The resulting mixture was heated at 100° C. for 4 h. The mixture was cooled down and subsequently separated with chromatography (hexane/ethyl acetate=20/1) to yield 4b as yellow solid (6.0 mg, 16.8% yield). ¹H NMR (500 MHz, DMSO-d₆) δ 7.18-7.11 (m, 5H), 7.07 (d, J=8.5 Hz, 2H), 7.00 (d, J=7.0 Hz, 2H), 6.90 (d, J=9.0 Hz, 2H), 6.81 (d, J=9.0 Hz, 2H), 6.71 (d, J=8.5 Hz, 2H), 6.64 (d, J=8.5 Hz, 2H), 3.68 (s, 3H), 3.64 (s, 3H), 3.26 (m, 1H), 1.08 (d, J=6.5 Hz, 6H); ¹³C NMR (125 MHz, DMSO-d₆) δ 168.7, 158.4, 146.5, 143.5, 141.6, 138.2, 135.5, 135.4, 132.6, 132.5, 132.3, 131.2, 131.1, 129.3, 128.4, 127.2, 113.6, 113.5, 85.9, 60.2, 55.4, 55.3, 49.0, 36.2, 29.4, 22.5, 20.5, 14.4; MS (ESI) calcd for [M+Na]⁺:533.22, found: 533.20.

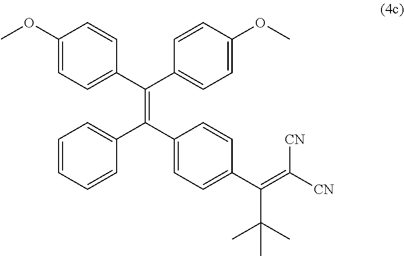 [SMILES: COc1ccc(C(=C(c2ccccc2)c2ccc(C(=C(C#N)C#N)C(C)(C)C)cc2)c2ccc(OC)cc2)cc1]

Compound 3c (34 mg, 0.06 mmol), malononitrile (15 mg, 0.20 mmol) and ammonium acetate (30 mg, 0.38 mmol) were dissolved in a mixture of dichloromethane (5 mL) and methanol (1 mL). Silica gel (475 mg) was then added to the above mixture, and the solvent was removed under reduced pressure. The resulting mixture was heated at 100° C. for 7.5 hours. The mixture was cooled down and subsequently separated with chromatography (hexane/ethyl acetate=20/1) to yield 4c as light yellow solid (9.0 mg, 33.3% yield). ¹H NMR (500 MHz, DMSO-d₆) δ 7.17 (m, 2H), 7.12 (m, 1H), 7.03-7.07 (m, 4H), 6.99 (dd, J, =1.5 Hz, J₂=8.5 Hz, 2H), 6.90 (d, J=9.0 Hz, 2H), 6.80 (d, J=8.5 Hz, 2H), 6.71 (d, J=9.0 Hz, 2H), 6.61 (d, J=8.5 Hz, 2H), 3.68 (s, 3H), 3.64 (s, 3H), 1.24 (s, 9H); ¹³C NMR (125 MHz, DMSO-d₆) δ 158.3, 158.2, 145.5, 143.5, 141.3, 138.3, 135.6, 135.5, 132.6, 132.5, 131.0, 128.4, 126.8, 126.2, 113.6, 113.5, 87.1, 55.4, 55.3, 29.3; MS (ESI) calcd for [M+Na]⁺: 547.23, found: 547.20.

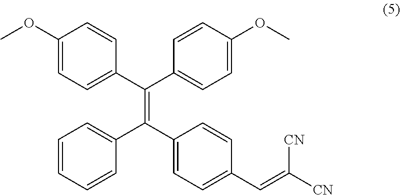 [SMILES: COc1ccc(C(=C(c2ccccc2)c2ccc(C=C(C#N)C#N)cc2)c2ccc(OC)cc2)cc1]

To the solution of compound 2 (87 mg, 0.2 mmol) in dichloromethane (5 mL was added malononitrile (25 mg, 0.8 mmol) and triethylamine (10 mg, 0.1 mmol). The resulting mixture was stirred at room temperature for 4 h. Then the solvent was removed under reduced pressure. The desired residue was purified with chromatography to yield the product as purple solid (79 mg, 85.0%). ¹H NMR (400 MHz, CDCl₃) δ 7.63 (d, J=8.4 Hz, 2H), 7.57 (s, 1H), 7.13-7.16 (m, 5H), 7.01 (m, 2H), 6.92-6.95 (m, 4H), 6.63-6.68 (m, 4H), 3.76 (s, 3H), 3.74 (s, 3H); ¹³C NMR (100 MHz, CDCl₃) δ 159.0, 158.8, 158.6, 152.0, 143.5, 143.1, 137.4, 135.4, 135.3, 132.7, 132.6, 132.5, 131.3, 130.3, 128.5, 128.0, 126.7, 114.1, 113.4, 113.0, 112.9, 80.8, 55.1, 55.0.

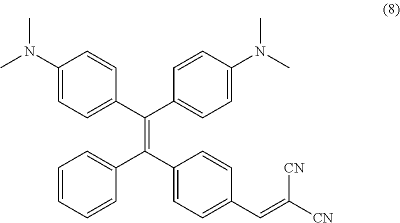 [SMILES: CN(C)c1ccc(C(=C(c2ccccc2)c2ccc(C=C(C#N)C#N)cc2)c2ccc(N(C)C)cc2)cc1]

To the solution of compound 2 (170 mg, 0.4 mmol) in ethanol (8 mL) was added malononitrile (54 mg, 0.8 mmol). The resulting mixture was refluxed for 12 h. Then the solvent was removed under reduced pressure. The desired residue was purified with chromatography to yield the product as purple solid (143 mg, 72.6%). ¹H NMR (400 MHz, CDCl₃) δ 7.65 (d, J=8.8 Hz, 2H), 7.60 (s, 1H), 7.10-7.16 (m, 5H), 7.04 (m, 2H), 6.90 (d, J₁=8.8 Hz, J₂=2.0 Hz, 4H), 6.48 (m, 4H), 2.93 (s, 6H), 2.90 (s, 6H); ¹³C NMR (100 MHz, CDCl₃) δ 159.1, 153.5, 149.5, 149.2, 145.3, 144.3, 134.9, 132.8, 132.7, 132.6, 131.6, 131.0, 130.3, 127.9, 127.8, 126.1, 114.4, 113.2, 111.3, 111.0, 79.6, 40.2.

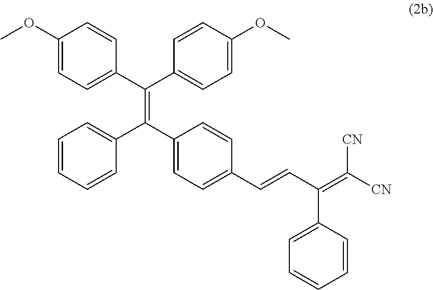 [SMILES: COc1ccc(C(=C(c2ccccc2)c2ccc(/C=C/C(=C(C#N)C#N)c3ccccc3)cc2)c2ccc(OC)cc2)cc1]

To the solution of compound 2a (0.18 g, 0.34 mmol) and malononitrile (30 mg, 0.45 mmol) in dichloromethane (10 mL) was added titanium tetrachloride (0.13 mL, 1.2 mmol) slowly at 0° C. After the reaction mixture was stirred for 30 min, pyridine (0.10 mL, 1.2 mmol) was injected and stirred for another 30 min. Then the mixture was heated at 40° C. for 4 h. After the mixture was cooled down to room temperature, the reaction was quenched by water (10 mL) and the mixture was extracted with dichloromethane. The collected organic layer was washed by brine (20 mL), dried over MgSO₄ and concentrated under reduced pressure. The desired residue was purified by column chromatography (hexane/ethyl acetate=50/1-10/1) to give the desired product as red solid (43 mg, 21.9% yield). ¹H NMR (400 MHz, CDCl₃) δ 7.49-7.66 (m, 4H), 7.36 (m, 2H), 7.27 (m, 2H), 7.11-7.17 (m, 4H), 6.99-7.05 (m, 4H), 6.91-6.95 (m, 4H), 6.80 (d, J=15.6 Hz, 1H), 6.62-6.69 (m, 4H), 3.73-3.77 (m, 6H).

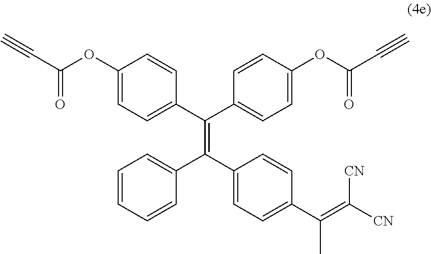 [SMILES: C#CC(=O)Oc1ccc(C(=C(c2ccccc2)c2ccc(C(C)=C(C#N)C#N)cc2)c2ccc(OC(=O)C#C)cc2)cc1]

To the solution of compound 4d (60 mg, 0.14 mmol) in dry dichloromethane (10 mL) was added propiolic acid (60 mg, 0.86 mmol), N,N′-dicyclohexylcarbidiimide (64 mg, 0.32 mmol) and dimethylaminopyridine (36 mg, 0.3 mmol) at −10° C. The reaction mixture was stirred at the same temperature for 1 h and then at room temperature for 1.5 h. The reaction was filtered to remove the un-dissolved solid and the filtrate was washed with water (20 mL) twice, brine (20 mL) once and dried with sodium sulfite. The organic phase was collected by filtration and concentrated under reduced pressure. The residue was purified with chromatography to yield the desired product 3 (15 mg, 19.2%) as yellow solid. ¹H NMR (400 MHz, CDCl₃): δ 7.33-7.35 (m, 2H), 7.11-7.16 (m, 5H), 6.98-7.05 (m, 6H), 6.91-6.95 (m, 4H), 3.06 (s, 1H), 3.04 (s, 1H), 2.58 (s, 3H); HRMS (ESI) calcd for [M+Na]⁺: 581.1477, found: 581.1483.

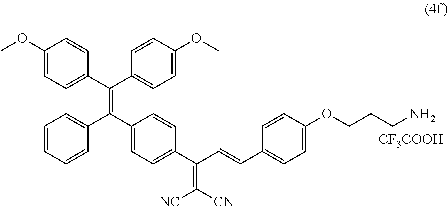 [SMILES: COc1ccc(C(=C(c2ccccc2)c2ccc(C(/C=C/c3ccc(OCCCN)cc3)=C(C#N)C#N)cc2)c2ccc(OC)cc2)cc1 ; "CF3COOH"]

To the solution of compound 4c (40 mg, 0,083 mmol) in isopropanol (5 ml) was added compound 1 (30 mg, 0.11 mmol) and piperidine (0.68 mg, 0.008 mmol). The resulting solution was refluxed for 24 hours. Then the solvent was removed under reduced pressure. The desired residue was purified with chromatography (hexane:ethyl acetate=5:1) to give a red oil. This oil was further treated with the mixture of dichloromethane (5 ml) and trifluoroacetic acid (1 ml) for 8 hours. The solvent was removed under reduced pressure. The residue was purified with reverse HPLC using acetonitrile and water as the mobile phase to give the desired product (yellow solid, 12 mg, 23.0%). ¹H NMR (400 MHz, DMSO-d₆) δ 7.79 (brs, 2H), 7.63 (d, J=8.8 Hz, 2H), 7.40 (d, J=15.2 Hz, 1H), 7.27 (d, J=8.4 Hz, 2H), 7.13-7.20 (m, 2H), 7.15 (m, 3H), 7.02-7.06 (m, 4H), 6.87-6.92 (m, 4H), 6.67-6.73 (m, 5H), 4.16 (d, J=6.0 Hz, 2H), 3.68 (s, 6H), 2.95-3.00 (m, 2H), 2.00-2.04 (m, 2H); ¹³C NMR (120 MHz, DMSO-d₆) δ 170.8, 161.3, 157.9, 148.5, 146.8, 142.9, 141.4, 137.8, 135.2, 135.0, 132.2, 132.0, 131.0, 130.8, 130.7, 128.6, 128.0, 126.9, 126.5, 121.9, 115.3, 113.2, 113.1, 79.2, 65.0, 54.9 (d), 26.7; MS (ESI) calcd for [M+H]⁺:644.2913, found: 644.2926.

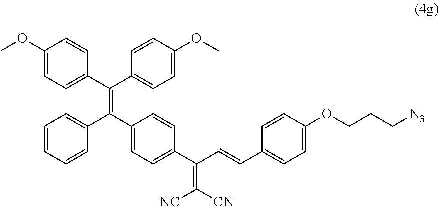 [SMILES: COc1ccc(C(=C(c2ccccc2)c2ccc(C(/C=C/c3ccc(OCCCN=[N+]=[N-])cc3)=C(C#N)C#N)cc2)c2ccc(OC)cc2)cc1]

To the solution of compound 4c (40 mg, 0.083 mmol) in isopropanol (5 ml) was added compound 1 (30 mg, 0.11 mmol) and piperidine (0.68 mg, 0.008 mmol). The resulting solution was refluxed for 24 hours. Then the solvent was removed under reduced pressure. The desired residue was purified with chromatography (hexane:ethyl acetate=5:1) to give the product as a red oil (15 mg, 27.3%). ¹H NMR (500 MHz, CDCl₃) δ 7.48 (d, J 9.0 Hz, 2H), 7.42 (d, J=15.5 Hz, 1H), 7.08-7.18 (m, 9H), 6.91-6.98 (m, 6H), 6.76 (d, J=15.5 Hz, 1H), 6.69 (d, J=8.5 Hz, 2H), 6.66 (d, J=8.5 Hz, 2H), 4.12 (t, J=6.0 Hz, 2H), 3.76 (s, 3H), 3.75 (s, 3H), 3.55 (t, J=6.5 Hz, 2H), 2.08 (m, 2H); ¹³C NMR (125 MHz, CDCl₃) δ171.2, 161.6, 158.5, 158.4, 148.8, 148.7, 143.2, 142.0, 138.0, 135.7, 135.5, 132.7, 132.5, 131.7, 131.3, 130.8, 128.4, 127.9, 127.4, 126.5, 122.3, 115.1, 113.2, 113.0, 80.1, 64.8, 55.2, 55.1, 48.0; 28.6.

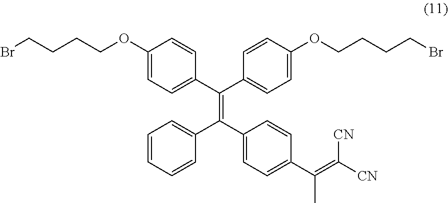 [SMILES: CC(=C(C#N)C#N)c1ccc(C(=C(c2ccc(OCCCCBr)cc2)c2ccc(OCCCCBr)cc2)c2ccccc2)cc1]

Compound 10 (20 mg, 0.03 mmol), malononitrile (21 mg, 0.32 mmol) and ammonium acetate (36 mg, 0.46 mmol) were dissolved in the mixture of dichloromethane (5 mL) and methanol (1 mL). Then silica gel (404 mg) was added to the above mixture, and the solvent was removed under reduced pressure. The resulting mixture was heated at 100° C. for 40 minutes. The mixture was cooled down and subsequently separated with chromatography (hexane/ethyl acetate (v/v)=20/1) to give the desired product as orange solid (16 mg, 74.0% yield). ¹H NMR (500 MHz, CDCl₃) δ 7.33 (d, J=8.5 Hz, 2H), 7.14 (m, 5H), 7.01 (m, 2H), 6.92 (dd, J₁=3.0 Hz, J₂=8.5 Hz, 4H), 6.64 (d, J=8.5 Hz, 2H), 6.62 (d, J=9.0 Hz, 2H), 3.93 (q, J=6.0 Hz, 4H), 3.48 (dt, J₁=3.0 Hz, J₂=7.0 Hz, 4H), 2.57 (s, 3H), 2.01-2.05 (m, 4H), 1.89-1.91 (m, 4H); ¹³C NMR (125 MHz, CDCl₃) δ 174.4, 157.9, 157.7, 149.2, 143.3, 142.5, 137.5, 135.6, 135.4, 133.0, 132.7, 132.6, 131.8, 131.3, 128.0, 126.9, 126.5, 113.8, 113.5, 70.5, 66.7, 66.6, 33.5, 33.4, 29.4, 27.8, 23.8; HRMS (ESI⁻) m/z: 721.1046 (Calcd for [M−H]⁻: 721.1071).

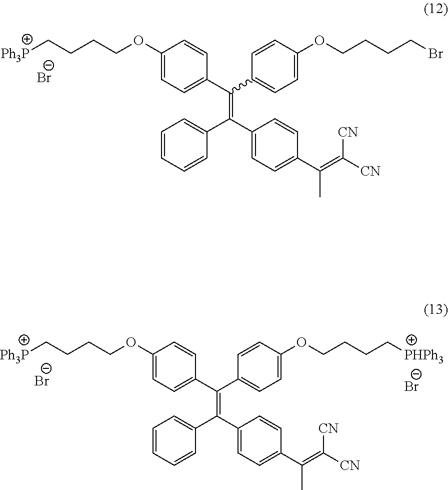

To the solution of compound 11 (16 mg, 0.022 mmol) in acetonitrile (5 mL) was added triphenylphosphine (64 mg, 0.24 mmol). The resulting mixture was refluxed for 48 hours. Then the solvent was removed under reduced pressure. The residue was washed with hexane (10 mL) and the remaining residue was purified with HPLC to give the product 12 (3 mg, orange oil), ¹H NMR (500 MHz, Methanol-d₄) δ 7.90 (q, J=7.0 Hz, 3H), 7.81-7.71 (m, 12H), 7.42 (d, J=8.5 Hz, 1H), 7.36 (d, J=8.5 Hz, 1H), 7.14 (m, 5H), 7.01 (d, J=7.0 Hz, 1H), 6.89-6.93 (m, 4H), 6.60-6.68 (m, 4H), 4.00 (q, J=5.5 Hz, 2H), 3.94 (m, 2H), 3.51 (q, J=7.0 Hz, 2H), 3.44 (m, 2H), 2.58 (s, 1.5H), 2.55 (s, 1.5H), 1.97-2.03 (m, 4H), 1.88 (m, 4H); HRMS (ESI) m/z: 905.2900 (Calcd for [M-Br]⁺: 905.2866); and 13 (5 mg, orange oil), ¹H NMR (500 MHz, DMSO-d₆) δ 7.90 (t, J=7.5 Hz, 6H), 7.80-7.71 (m, 24H), 7.47 (d, J=8.5 Hz, 6H), 7.06-7.17 (m, 3H), 7.07 (d, J=8.0 Hz, 2H), 6.98 (d, J=7.0 Hz, 2H), 6.86 (dd, =2.5 Hz, J₂=8.5 Hz, 4H), 6.65 (d, J=8.0 Hz, 4H), 3.95-3.90 (m, 4H), 2.53 (s, 3H), 1.86 (m, 4H), 1.66 (m, 4H); HRMS (ESI) m/z: 544.2281 (Calcd for [M-2Br]²⁺: 544.2294).

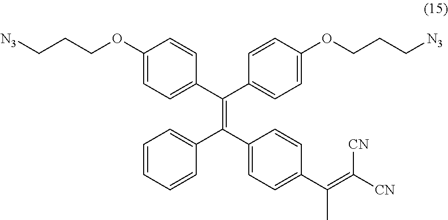 [SMILES: CC(=C(C#N)C#N)c1ccc(C(=C(c2ccc(OCCCN=[N+]=[N-])cc2)c2ccc(OCCCN=[N+]=[N-])cc2)c2ccccc2)cc1]

Compound 14 (25 mg, 0.05 mmol), malononitrile (15 mg, 0.20 mmol) and ammonium acetate (20 mg, 0.26 mmol) were dissolved in the mixture of dichloromethane (5 mL) and methanol (1 mL). Then silica gel (300 mg) was added to the above mixture. After the solvent was removed under reduced pressure, the resulting mixture was heated at 100° C. for 40 minutes. The mixture was cooled down and subsequently separated with column chromatography (hexane/ethyl acetate=20/1) to give the desired product (19 mg, 61.2% yield) as a reddish orange oil. ¹H NMR (500 MHz, DMSO-d₆) δ 7.50 (d, J=8.5 Hz, 2H), 7.09-7.19 (m, 5H), 7.00 (m, 2H), 6.89 (dd, J₁=2.0 Hz, J₂=8.5 Hz, 4H), 6.70-6.73 (m, 4H), 3.96 (t, J=6.0 Hz, 4H), 3.49 (dt, J₁=2.5 Hz, J₂=6.5 Hz, 4H), 2.56 (s, 3H), 1.91-1.96 (m, 4H); ¹³C NMR (125 MHz, DMSO-d₆) δ 176.4, 157.7, 157.5, 148.5, 143.5, 142.1, 138.0, 135.7, 135.6, 133.8, 132.5, 132.4, 131.4, 131.2, 128.5, 127.9, 127.0, 114.3, 114.1, 113.9, 82.9, 64.9, 48.1, 28.6, 28.5, 24.3; HRMS (EI) calcd. for [M]⁺: 620.2648, found: 620.2634.

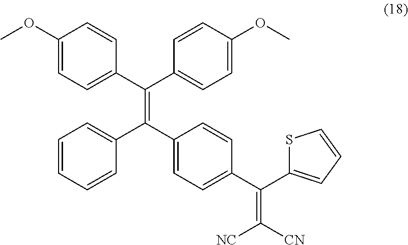 [SMILES: COc1ccc(C(=C(c2ccccc2)c2ccc(C(=C(C#N)C#N)c3cccs3)cc2)c2ccc(OC)cc2)cc1]

To the solution of compound 17 (0.26 g, 0.52 mmol) and malononitrile (45 mg, 0.68 mmol) in dichloromethane (10 mL) was added titanium tetrachloride (0.20 mL, 1.8 mmol) slowly at 0° C. After the reaction mixture was stirred for 30 min, pyridine (0.15 mL, 1.8 mmol) was injected and stirred for another 30 min. Then the mixture was heated at 40° C. for 4 h. After the mixture was cooled down to room temperature, the reaction was quenched by water (10 mL) and the mixture was extracted with dichloromethane. The collected organic layer was washed by brine (20 mL), dried over MgSO₄ and concentrated under reduced pressure: The desired residue was purified by column chromatography (hexane/ethyl acetate=50/1-10/1) to give the desired product as red solid (230 mg, 81.0% yield). ¹H NMR (400 MHz, CDCl₃) δ 7.80 (dd, J₁=1.2 Hz, J₂=5.2 Hz, 1H), 7.73 (dd, J₁=1.2 Hz, J₂=5.2 Hz, 1H), 7.13-7.22 (m, 8H), 7.06 (m, 2H), 8.91-8.98 (m, 4H), 8.64-8.68 (m, 4H), 3.75 (s, 6H); ¹³C NMR (100 MHz, CDCl₃) δ 164.8, 158.6, 158.4, 148.7, 143.2, 142.4, 138.7, 137.7, 136.1, 135.7, 135.5, 133.5, 132.6, 132.5, 131.5, 131.3, 129.1, 128.8, 127.9, 126.5, 114.5, 113.8, 113.2, 113.0, 55.1, 55.0. MS (EI) calcd for [M]⁺: 550.1709, found: 550.1708.

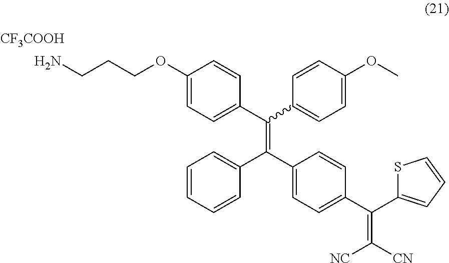 [SMILES: "CF3COOH" ; COc1ccc(C(=C(c2ccccc2)c2ccc(C(=C(C#N)C#N)c3cccs3)cc2)c2ccc(OCCCN)cc2)cc1]

To the solution of compound 20 (28 mg, 0.04 mmol) in dichloromethane (5 mL) was added trifluoroacetic acid (1 mL). The resulting mixture was stirred at room temperature for 6 h. Then the mixture was concentrated under reduced pressure to give the product (10.0 mg as red solid, 43.4% yield): ¹H NMR (500 MHz, CDCl₃) δ 8.27 (dd, J₁=1.0 Hz, J₂=5.0 Hz, 1H), 7.77 (brs, 3H), 7.67 (dd, J₁=1.5 Hz, J₂=4.0 Hz, 1H), 7.38 (dd, J₁=4.0 Hz, J₂=5.0 Hz, 1H), 7.35 (m, 2H), 7.17-7.20 (m, 2H), 7.10-7.14 (m, 3H), 7.01-7.03 (m, 2H), 6.84-6.90 (m, 4H), 6.68-6.72 (m, 4H), 3.96 (t, J=6.0 Hz, 2H), 3.68 (s, 3H), 2.95 (m, 2H), 1.98 (m, 2H); HRMS (ESI) calcd for [M+H]⁺: 594.2210, found: 594.2215.

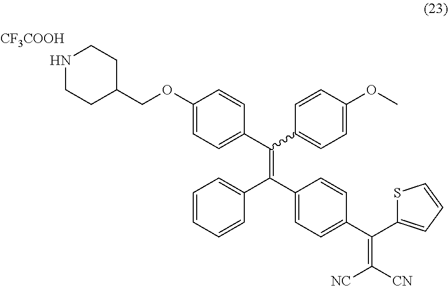 [SMILES: "CF3COOH" ; COc1ccc(C(=C(c2ccccc2)c2ccc(C(=C(C#N)C#N)c3cccs3)cc2)c2ccc(OCC3CCNCC3)cc2)cc1]

To the solution of compound 22 (30 mg, 0.04 mmol) in dichloromethane (5 mL) was added trifluoroacetic acid (1 mL). The resulting mixture was stirred at room temperature for 6 h. Then the mixture was concentrated under reduced pressure to give the product (17.0 mg as red solid, 56.6% yield). MS (ESI) calcd for [M+H]⁺: 633.24, found: 634.20.

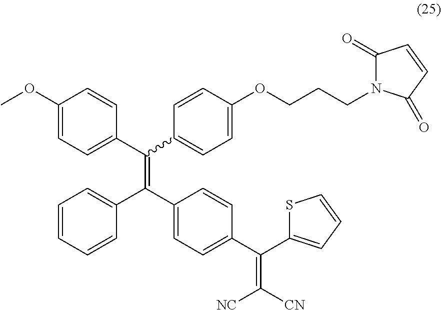 [SMILES: COc1ccc(C(=C(c2ccccc2)c2ccc(C(=C(C#N)C#N)c3cccs3)cc2)c2ccc(OCCCN3C(=O)C=CC3=O)cc2)cc1]

Compound 24 (48 mg, 0.064 mmol) was dissovled in toluene (10 mL). The resulting solution was refluxed for 24 h. Then the solvent was removed under reduced pressure. The desired residue was purified with chromatography (hexane/ethyl acetate=50/1-5/1) to give the desired product as red solid (36 mg, 83.7%). HRMS (ESI) calcd for [M+Na]+: 696.1927, found: 696.1937.

¹O₂ Quantum Yield Measurements

The ¹O₂-sensitive indicator, 9,10-anthracenediyl-bis(methylene) dimalonic acid (ABDA), was used as the ¹O₂-trapping agent, and Rose Bengal (RB) was used as the standard photosensitizer. In these experiments, 10 μL of ABDA solution (2 M) was added to 1 mL of sample solution, and white light (400-800 nm) with a power density of 0.25 W cm⁻² was employed as the irradiation source. The absorbance of ABDA at 378 nm was recorded at different irradiation time to obtain the decay rate of the photosensitizing process. The ¹O₂ quantum yield of the PS in water (Φ_{PS}) was calculated using the following formula:

$${Φ}_{PS} = {Φ}_{RB} \frac{{K}_{PS} * {A}_{RB}}{{K}_{RB} * {A}_{PS}} \qquad ( 2 )$$

Where K_{PS} and K_{RB} are the decomposition rate constants of ABDA by the PSs and RB, respectively. A_{PS} and A_{RB} represent the light absorbed by the PSs and RB, respectively, which are determined by integration of the optical absorption bands in the wavelength range 400-800 nm. Φ_{RB} is the ¹O₂ quantum yield of RB, and Φ_{RB}=0.75 in water.

To assess capabilities of PPDC, TPPDC, and TPDC in ¹O₂ generation, a commercial ¹O₂ probe ABDA was used as an indicator and Rose Bengal (RB) was used as the standard photosensitizer (¹O₂ quantum yield O_{RB}=0.75 in water). In the presence of PSs or RB under irradiation with white light, the absorbance of the ABDA solution at 378 nm, decreases with prolonged irradiation time, indicating the degradation of ABDA by ¹O₂ generated by PSs. Among these compounds, PPDC exhibited the largest degradation rate of ABDA (0.0032), of which for TPPDC and TPDC is 0.0018 and 0.0013, with a smallest absorption integrated area (4.68) in white light region. Thus, the ¹O₂ quantum yield of PPDC, TPPDC and TPDC was calculated to be 0.89, 0.32 and 0.28, respectively. These findings agree well with the prediction based on eq. (1).

### Photodynamic Therapy.

Low cytotoxicity in dark conditions but high toxicity upon exposure to light irradiation is useful for particle use of phototherapy. Quantitative evaluation of the therapeutic effect of TAT-TPDC NPs and TAT-PPDC NPs was studied by standard MTT assay. The cytotoxicity of HeLa cells upon incubation with TAT-TPDC NPs and TAT-PPDC NPs in dark conditions was first evaluated. After 24 h incubation, no significant cytotoxicity is observed in dark. However, after exposure to light irradiation, a dose-dependent cytotoxicity is observed in HeLa cells. The half-maximal inhibitory concentrations (IC₅₀) of TAT-TPDC NPs and TAT-PPDC NPs for HeLa cells are 3.44 and 1.28 μg mL⁻¹, respectively. The lower IC₅₀ of TAT-PPDC NPs relative to that for TAT-TPDC NPs can be attributed to more ROS generation upon light irradiation. Although the difference is not as significant as that in the solution study, the 2.6-fold lower of IC₅₀ of TAT-TPDC NPs is reckoned considerable in cancer cell inhibition. Furthermore, to validate the exposure time and light power dependent PDT, the TAT-TPDC NPs and TAT-PPDC NPs incubated HeLa cells were irradiated with light for different time durations or at different power densities. Enhanced inhibition of cell viability is observed as a result of longer laser irradiation time or higher light power density for both NPs. These results indicate that the therapeutic efficiency can be regulated by controlling the laser irradiation time or the light power density. Furthermore, TAT-PPDC NPs showed stronger inhibition of cell viability than TAT-TPDC NPs in both cases.

The apoptosis pathway of TAT-TPDC NPs and TAT-PPDC NPs treated HeLa cells after light exposure was then studied by costaining with Fluorescein isothiocyanate (FITC)-tagged Annexin V. FITC-tagged Annexin V is commonly used to distinguish viable cells from apoptotic ones as the Annexin V can selectively bind to the exposed phosphatidylserines on the outer cytoplasmic membrane of apoptotic cells. After incubation of HeLa cells with TAT-TPDC NPs or TAT-PPDC NPs followed by light irradiation and FITC-tagged Annexin V costaining, strong green fluorescence attributed to FITC is clearly observed in cell membranes, indicating the cells undergoing apoptosis process. On the other hand, no green fluorescence signal is observed in the same HeLa cells in dark conditions, indicating the TAT-TPDC NPs and TAT-PPDC NPs do not cause observable cell toxicity.

### Example 2

Specific Light-Up Bioprobe with AIE and Activatable Photoactivity for the Targeted and Image-Guided Photodynamic Ablation of Cancer Cells

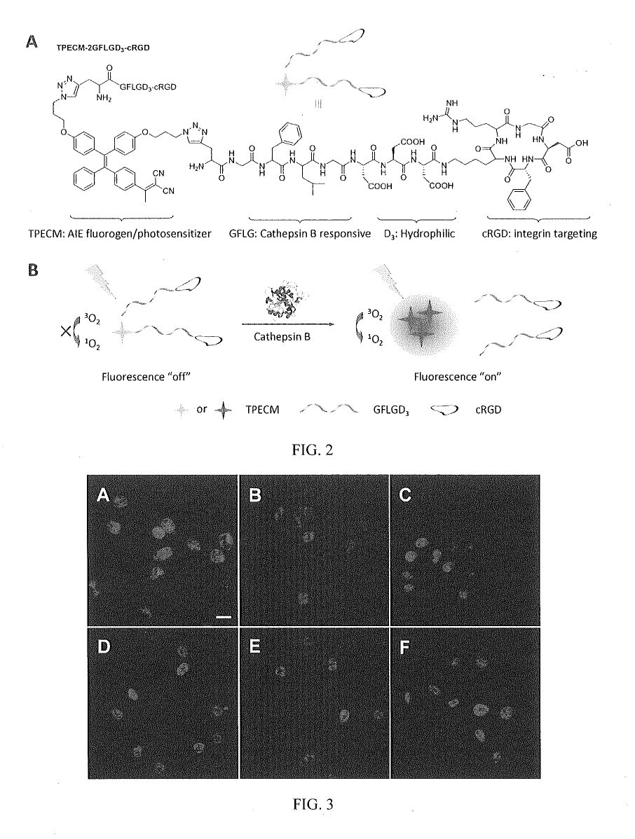

In another example embodiment, the present invention is an activatable photosensitizer illustrated in FIG. 2A useful for image-guided photodynamic ablation of cancer cells. FIG. 2A illustrates a synthetic route to the functionalizable TPE derivative TPECM-2N3 and the bioprobe TPECM-2GFLGD3-cRGD.

Cathepsin B is a lysosomal protease overexpressed in many types of tumors. It can specifically cleave substrates with a -Gly-Phe-Leu-Gly-(GFLG) peptide sequence and has been used for enzyme-responsive drug delivery. On the other hand, cyclic arginine-glycine-aspartic acid (cRGD), which can selectively interact with avb3 integrin overexpressed in cancer cells, has been used for targeted drug delivery.

In an example embodiment, the probe is composed of four parts: 1) an orange fluorescent AIE fluorogen as an imaging reagent and photosensitizer, 2) a GFLG peptide substrate that is responsive to cathepsin B, 3) a hydrophilic linker with three Asp (D) units to increase the hydrophilicity of the probe, and 4) a cRGD-targeting moiety. This probe is referred to as Fluorogen 1. The probe is almost nonfluorescent with a very low ROS-generation ability in aqueous media owing to the consumption of excitonic energy by free intramolecular motions. After cancer-cellular uptake, cleavage of the GFLG substrate by cathepsin B will lead to enhanced fluorescence signal output concomitant with activated photoactivity for image-guided PDT. Therefore, the probe design offers a good opportunity to develop activatable PSs without incorporating any quencher or energy acceptor. Enhanced fluorescence and phototoxicity is then observed in the aggregate state upon activation by tumor-related stimuli. FIG. 2B illustrates probe activation by cathepsin B with fluorescence “turn-on” and activated photoactivity to generate reactive oxygen species (ROS) upon irradiation with light.

Fluorogen 1 shows orange-red emission in aggregates and can be excited by both 405 and 457 nm lasers. ROS generation of the AIE fluorogen 1 upon irradiation with light by using 1,3-diphenylisobenzofuran (DPBF) and 2′,7′-dichlorodihydrofluorescein diacetate (DCFDA) as the ROS indicators was then studied. DPBF can readily undergo 1,4-cycloaddition reactions with ROS, which results in decreased absorbance at 418 nm, whereas DCFDA is nonfluorescent but can be rapidly oxidized by ROS to the fluorescent molecule dichlorofluorescein (DCF).

To demonstrate cell-specific light-up imaging, the probe was incubated with MDA-MB-231 cells overexpressing avb3 integrin and used MCF-7 and 293T cells as negative controls. Upon incubation with the probe, the red fluorescence in MDA-MB-231 cells intensified gradually as the incubation time increased (as seen in FIG. 3A). The specific fluorescence light-up of the probe in cells was also confirmed by flow cytometry analysis, which revealed receptor-mediated probe uptake by MDA-MB-231 cells. Furthermore, the fluorescence intensity in the cells intensified when the probe was incubated at a higher concentration, thus indicating the potential for semiquantification of the activated AIE probe inside cells, as illustrated in FIG. 3A-C.

FIGS. 3 A-F are confocal images of A) MDA-MB-231 cells (colored blue and red), B) MCF-7 cells (colored only blue), C) 293T cells (colored only blue), D) MDA-MB-231 cells pretreated with free cRGD (colored blue and red), E) MDA-MB-231 cells pretreated with CA-074-Me (colored blue and red), and F) MDA-MB-231 cells pretreated with both cRGD and CA-074-Me after incubation with the probe (5 mm) for 4 h (colored only blue). The blue fluorescence is from the cell nuclei dyed with 4′,6-diamidino-2-phenylindole (DAPI; Ex=405 nm; Em=430-470 nm), the red fluorescence is from the probe (Ex=405 nm; Em>560 nm). All images share the same scale bar (20 mm).

### Example 3

Image-Guided Combination Chemotherapy and Photodynamic Therapy Using a Mitochondria-Targeted Molecular Probe with AIE Induced Emission Characteristics

In another example embodiment, the present invention is AIE probe with zero, one or two triphenylphosphine ligands, the probe being able to selectively target the mitochondria. An example embodiment of a probe with zero PPh₃ ligands is TPECM-2Br, which is represented by the following structure:

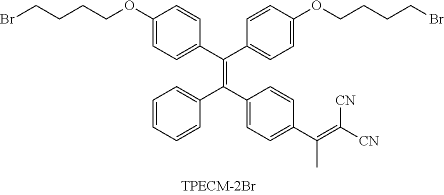 [SMILES: CC(=C(C#N)C#N)c1ccc(C(=C(c2ccc(OCCCCBr)cc2)c2ccc(OCCCCBr)cc2)c2ccccc2)cc1]

An example of a probe with one PPh₃ ligand is TPECM-1TPP, represented by the following structure:

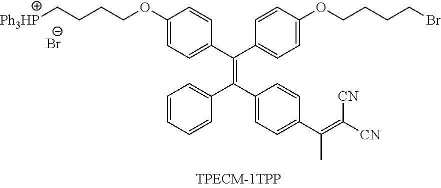

An example of a probe with two PPh₃ ligands is TPECM-2TPP, represented by the following structure:

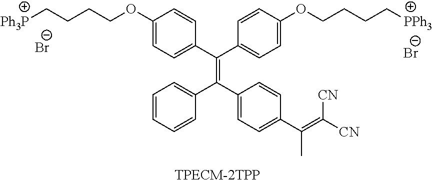

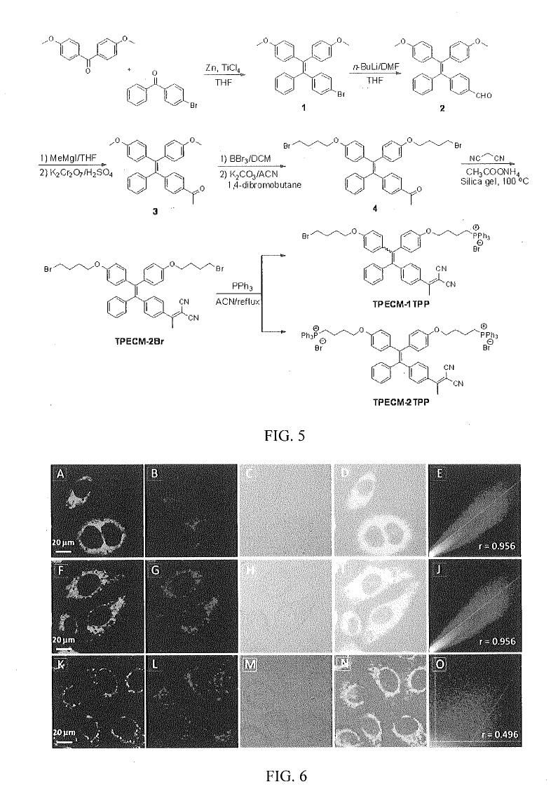

A synthetic route to the above compounds can be seen in FIG. 5.

Lipophilic triphenylphosphonium as a mitochondria targeting moiety was selected to conjugate to TPECM-2Br because it possesses a delocalized positive charge and can selectively accumulate in cancer cell mitochondria by trans-membrane potential gradient. The obtained TPECM-1TPP and TPECM-2TPP are almost non-emissive in aqueous media, but they emit strong red fluorescence in aggregated state. TPECM-2TPP is found to be able to depolarize mitochondria membrane potential and selectively exert potent chemo-cytotoxicity on cancer cells. Furthermore, the probe can efficiently generate reactive singlet oxygen with strong photo-toxicity upon light illumination, which further enhances the anti-cancer effect.

The probes of TPECM-2Br, TPECM-1TPP and TPECM-2TPP were synthesized according to FIG. 4C. Two different benzophenone derivatives were, reacted in the presence of Zn and TiCl₄ to give 1 in 27.2% yield, which was subsequently treated with n-BuLi and DMF to give 2 in 59.7% yield. 2 was first reacted with the Grignard reagent and the resulted secondary alcohol was further oxidized to generate 3 in 61.5% yield. 3 was subsequently treated with boron tribromide, followed by reaction with 4-dibromobutane to give 4 in 13.5% yield. The mixture of 4, ammonium acetate and malononitrile adsorbed on silica gel was heated at 100° C. for 40 minutes to give TPECM-2Br in 74.0% yield, which was then reacted with triphenylphosphine to generate TPECM-1TPP in 13.8% yield and TPECM-2TPP in 18.2% yield. The purified intermediates and products were well characterized by NMR and mass spectroscopies which confirmed their right structures with high purity.

The photophysical properties are as follows for TPECM-2Br. TPECM-2Br has an absorption maximum at 410 nm in DMSO/water (v/v=1:199). The photoluminescence (PL) spectra of TPECM-2Br were studied in DMSO/water mixtures with different water fractions (f_{w}). TPECM-2Br is faintly fluorescent in DMSO. However, with gradual increasing f_{w}, TPECM-2Br becomes highly emissive with an emission maximum at 628 nm, showing a characteristic AIE phenomenon. TPECM-1TPP and TPECM-2TPP in DMSO/water (v/v=1:199) showed similar absorption profiles to that of TPECM-2Br. However, their emission spectra in water are very different. To test the AIE characteristics of TPECM-1TPP and TPECM-2TPP, the mixtures of hexane and isopropyl alcohol were applied to study their fluorescent signals. TPECM-1TPP and TPECM-2TPP become highly emissive when the volume fraction of hexane is gradually increased to more than 80% and the nano-aggregates formation was also confirmed by laser light scattering (LLS). These results indicate that all the three probes are AIE active.

Additionally, TPECM-1TPP was also found to be able to visualize the mitochondria morphological changes under high oxidative stress induced by light-irradiation. Under the dark condition, mitochondria in TPECM-1TPP-treated cells were tubular-like. But after white light irradiation, mitochondria adopted small round shapes. The swelling of mitochondria is another evidence to indicate the depolarization of the mitochondrial membrane potential. As such, TPECM-1TPP is not only a good PS, but also an imaging tool to monitor the mitochondria morphological change during PDT.

FIG. 6 illustrates confocal images of HeLa cells after incubation with 2 μM TPECM-1TPP (A-D), TPECM-2TPP (F-I) and TPECM-2Br (K-N), co-stained with 100 nM Mito-tracker green. The green fluorescence in FIGS. 6A, 6F, and 6K, is from Mito-tracker green, λₑₓ=488 nm and λₑₘ=520 nm±20 nm, the red fluorescence in FIGS. 6B, 6G, and 6L is from the probes, λₑₓ=405 nm, λₑₘ>560 nm long pass filter. All images share the same scale bar of 20 μm. Co-localization scatter plots for TPECM-1TPP (E), TPECM-2TPP (J) and TPECM-2Br (O) in mitochondria of HeLa cells.

### Intracellular Localization of TPECM-2Br, TPECM-1TPP and TPECM-2TPP

HeLa cells were cultured in the chambers (LAB-TEK, Chambered Coverglass System) at a density of 5×10⁵ per mL for 18 h. The culture medium was removed, and the cells were rinsed with PBS. HeLa cells were incubated with TPECM-2Br (2 μM), TPECM-1TPP (1, 2 and 5 μM), TPECM-2TPP (1, 2 and 5 μM) at 37° C. for 3 h. For co-localization study, cells were washed with PBS, 200 nM of Mito-Tracker green was added and incubated at 37° C. for 45 min. After washing with PBS for 3 times, cells were placed on ice and imaged by confocal laser scanning microscope (CLSM, Zeiss LSM 410, Jena, Germany). For TPECM-2Br, TPECM-1TPP and TPECM-2TPP, the excitation was 405 nm, and the band filter was 560 nm; for Mito-Tracker imaging, the excitation was 488 nm, and the emission filter was 510-560 nm.

To study photo-induced mitochondria morphology change, the MDA-MB-231 cells were cultured in the chamber at a density of 5×10⁵ per mL for 18 h. After incubation with 5 μM of TPECM-1TPP for 3 h in the dark, the cells were irradiated for 8 min at the power density of 0.25 W cm⁻². Then the cells were stained with 200 nM Mito-Tracker green at 37° C. for 45 min and immediately imaged by confocal laser scanning microscope (CLSM, Zeiss LSM 410, Jena, Germany).

FIG. 7 illustrates the mitochondrial morphology change of MDA-MB-231 cells after treatment with TPECM-1TPP (5 μM) under dark (A-C) or light irradiation (0.1 W cm⁻², 8 min) (D-F). A and D are images from Mito-tracker green, λₑₓ=488 nm; λₑₘ=520 nm±20 nm. B and E are images from TPECM-1TPP, λₑₓ=405 nm; λₑₓ>560 nm long pass filter (colored red). C and F are overlay images from Mito-tracker green and TPECM-1TPP (colored yellow).

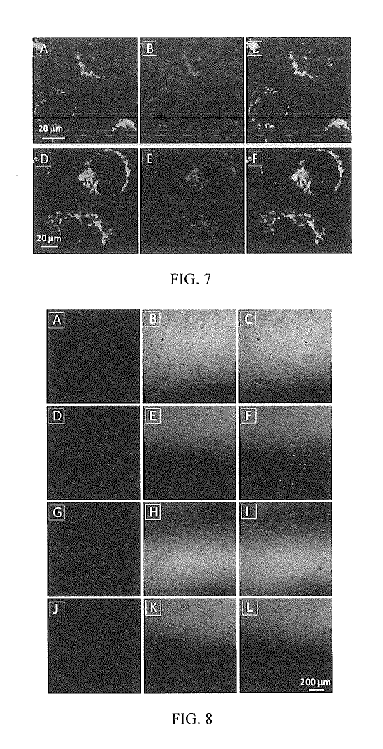

FIG. 8 is confocal fluorescence (A, D, G and J), bright field (B, E, H and K) and overlay fluorescence and bright field (C, F, I and L) images of PI stained HeLa cells after incubation of the cells without TPECM-2TPP (A, B and C), or with TPECM-2TPP (1 μM) in dark for 24 h (D, E and F) or with TPECM-2TPP (1 μM) for 3 h in dark followed by washing-away of the probe, white light irradiation (8 min, 0.10 W cm²) and further incubation for 24 h (G, H and I) or with TPECM-2TPP (1 μM) for 3 h in dark followed by washing-away of the probe, pre-incubation with Vitamin C (100 μM, 15 min), white light irradiation (8 min, 0.10 W cm⁻²) and further incubation for 24 h (J, K and L).

### Example 4

### Photoactivatable AIE Polymer: Concurrent Endo-/Lysomal Escaping and DNA Unpacking

In another embodiment, a ROS-responsive polymer for image-guided and spatiotemporally controlled gene delivery was developed. The polymer contains an AIE PS conjugated with oligoethylenimine (OEI) (800 Da) via a ROS-cleavable aminoacrylate (AA) linker. Low-molecular-weight OEIs were selected as the arm because they have reduced toxicity than high-molecular-weight PEI, and the OEI conjugates have shown good DNA binding ability. PEG was further grafted to fine-tune the water-solubility of the polymer. The polymer can self-assemble into nanoparticles (NPs) in aqueous media with bright red fluorescence, which bind to DNA through electrostatic interactions. Upon single light irradiation, the generated ROS can facilitate the vectors to escape from endo-/lysosomes by disruption its membrane. Concurrently, the ROS also breaks the polymer and promotes reversion of the high molecular weight complex back to their low molecular weight counterparts, leading to quick DNA unpacking. This work represents a promising spatiotemporally controlled and image-guided platform for concurrent endo-/lysosomal escaping and DNA unpacking, which are indispensable steps for efficient gene delivery.

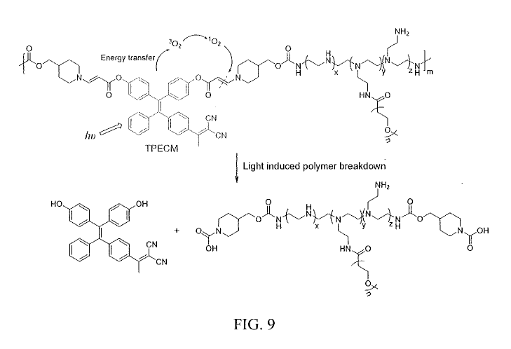

A proposed synthetic route to the ROS-responsive polymer, which is not intended to be limiting in theory is shown in FIG. 9. Under light illumination, TPECM can generate ROS to cleave the ROS-responsive linker, leading to breakdown of the polymer. The amphiphilic P(TPECM-AA-OEI)-g-mPEG can self-assemble in aqueous media to form ROS sensitive NPs (denoted as S-NPs), which were studied by dynamic light scattering (DLS) and transmission electron microscopy (TEM). S-NPs show spherical morphology with a diameter of 134±12 nm. The absorption and emission spectra of S-NPs are centered at 410 nm and 615 nm, respectively. The control polymer P(TPECM-OEI)-g-mPEG was synthesized without the ROS responsive linker, and the self-assembled NPs=denoted as inS-NPs.

The ROS generation of S-NPs and inS-NPs upon light irradiation was evaluated using dichlorofluorescein diacetate (DCF-DA) as an indicator. DCF-DA is non-fluorescent, but it can be rapidly oxidized by ROS to yield fluorescent dichlorofluorescein (DCF).

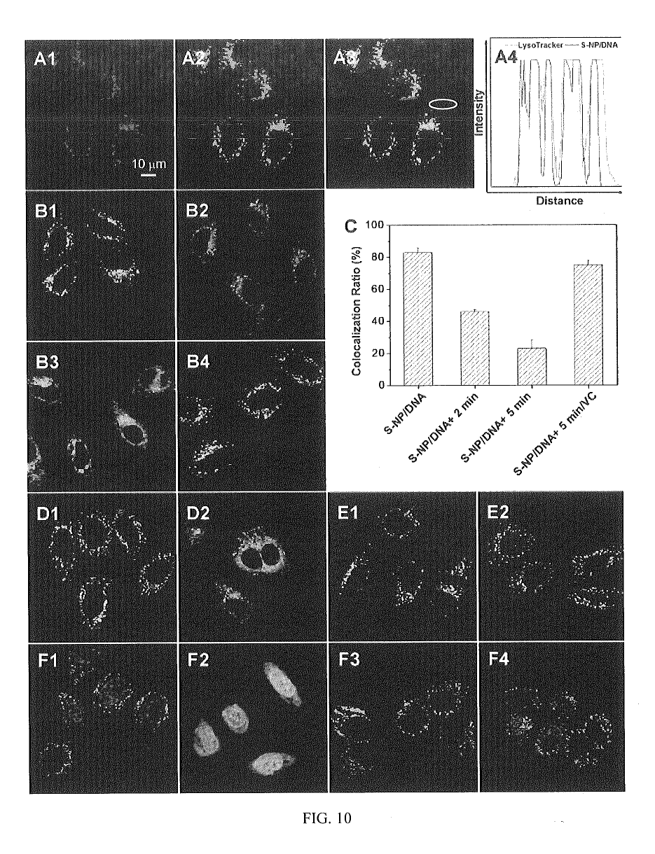

FIGS. 10A-F4 illustrate (A) CLSM images of HeLa cells stained with S-NPs/DNA (A1, Eₓ: 405 nm, Eₘ: >560 nm) and LysoTracker green (A2, Eₓ: 488 nm, Eₘ: 505-525 nm); (A3) overlay of the images A1 and A2; (A4) intensity profiles of region of interest (circled area in image A3). (B) CLSM images of HeLa cells incubated with S-NPs/YOYO-1-DNA complexes (B1) in dark, with light irradiation for (B2) 2 min, (B3) 5 min and (B4) 5 min in the presence of VC. Green: YOYO-1 fluorescence (Eₓ: 488 nm; Eₘ: 505-525 nm); Red: S-NPs fluorescence (Eₓ: 405 nm; Eₘ: >560 nm). Yellow: co-localization of red and green pixels. (C) Changes in co-localization ratios between the fluorescence of YOYO-1 and S-NPs after different treatment. (D, E) CLSM images of HeLa cells incubated with (D)S-NPs/YOYO-1-DNA pretreated with chloroquine (CQ), (E) inS-NPs/YOYO-1-DNA in dark (D1, E1) or with 5 min light irradiation (D2, E2). (F) CLSM images illustrating localization of YOYO-1-DNA after different treatments with further 4 h incubation. S-NPs/DNA in dark (F1), S-NPs/DNA with light irradiation (F2), S-NPs/DNA in the presence of VC with light irradiation (F3) and inS-NPs/DNA with light irradiation (F4). Green: YOYO-1 fluorescence (Eₓ: 488 nm; Eₘ: 505-525 nm); Red: nuclei living stained with DRAQ5 (Eₓ: 633 nm; Eₘ: >650 nm); Yellow: co-localization of red and green pixels. All images share the same scale bar of 10 μm.

The intracellular trafficking profile of S-NPs/DNA complexes was subsequently evaluated by confocal laser scanning microscopy (CLSM). Human cervix carcinoma HeLa cells were incubated with S-NPs/DNA for 4 h and co-stained with endo-/lysosome selective marker LysoTracker green DND-26. As shown in FIGS. 10A3 and 10A4, the red fluorescence from the complex co-localizes well with the green fluorescence from DND-26, indicating that the complexes are entrapped in endo-/lysosomes.

The ROS generation of S-NPs/DNA in HeLa cells was first confirmed by using DCF-DA as the indicator. When the cells were incubated with S-NPs/YOYO-1-DNA in dark, the red fluorescence of S-NPs and green fluorescence of YOYO-1 labeled DNA are largely overlaid as yellow dots (FIG. 10B1). However, upon light irradiation, the cells exhibit notably enhanced separation of green fluorescence from the red (FIG. 10B), indicating light induced intracellular DNA release. The unpacked DNA strains spread to the entire cytoplasm, which is indicates successful escape from the endo-/lysosomes.

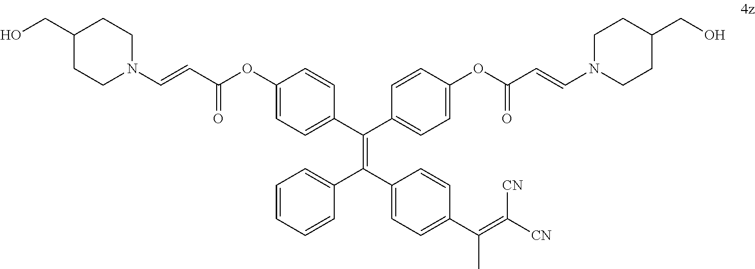 [SMILES: CC(=C(C#N)C#N)c1ccc(C(=C(c2ccc(OC(=O)/C=C/N3CCC(CO)CC3)cc2)c2ccc(OC(=O)/C=C/N3CCC(CO)CC3)cc2)c2ccccc2)cc1]

To the solution of compound 4e (above) (0.054 mmol) in THF (0.75 mL) was added 4-piperidinemethanol (12.3 mg, 0.108 mmol). The mixture was stirred at room temperature for 1 h and used directly in the next step without further purification. HRMS (ESI) calcd for [M+Na]+: 811.3472, found: 811.3492.

### Synthesis of the Polymer P(TPECM-AA-OEI)-g-mPEG.

The polymer was prepared according to a similar procedure reported before. Compound 4z (10 mg, 12.7 μmol) and CDI (8.2 mg, 50.7 μmol) were dissolved in 0.2 mL of anhydrous DMF. The mixture was stirred at room temperature for 1 h under nitrogen and then precipitated into cold diethyl ether twice. The resulting product was centrifuged, redissolved in 1 mL of anhydrous DMSO and added quickly to the solution of OEI800 (7.6 mg, 12.7 μmol) in DMSO (1 mL) in the presence of DIPEA (10 μL) with vigorous stirring. After the reaction was conducted for 5 h, MPEG-NHS (12.7 mg, 6.3 μmol) in anhydrous DMSO (0.5 mL) was added under N2 atmosphere and the mixture was stirred at room temperature for 24 h. After the reaction, the mixture was dialyzed (molecular weight cutoff of 8,000 Da, Spectrum Laboratories, Rancho Dominguez, Calif., USA) against deionized (DI) water. The polymer P(TPECM-AA-OEI)-g-mPEG was obtained as yellow powder after freeze drying (13.3 mg, 43%).

DNA Unpacking from S-NPs/DNA (N/P Ratio of 20) Studied by YOYO-1.

DNA was first labeled with the intercalating dye YOYO-1 iodide at a dye/base pair ratio of 1:50 and incubated at room temperature for 2 h.4 The complexes were formed at an N/P ratio of 20 by complexing YOYO-1 labeled DNA with the nanoparticles. The complexes were then transferred to a quartz cuvette and irradiated with white light (50 mW cm-2) for specific periods of time. The fluorescence of YOYO-1 after different duration of light irradiation was measured upon excitation at 488 nm and the emission was collected at 509 nm. The fluorescence of YOYO-1 in S-NPs/DNA after different time of light irradiation was then compared to the fluorescence intensity of free YOYO-1 labeled DNA.

Detection of ROS Generation from the Nanoparticles in Solution.

A ROS-sensitive indicator, dichlorofluorescein diacetate (DCF-DA), was used in our experiment to detect the ROS generation upon light irradiation according to a reported procedure.5 Briefly, to convert dichlorofluorescein diacetate (DCF-DA) to dichlorofluorescein, 0.5 mL of 1 mM DCF-DA in ethanol was added to 2 mL of 0.01 N NaOH and allowed to stir at room temperature for 30 min. The hydrolysate was then neutralized with 10 mL of 1×PBS at pH 7.4, and stored on ice until use. The nanoparticles in the above solution (0.1 mg mL-1) were exposed to light irradiation for different time intervals at a power density of 50 mW cm-2. The fluorescence change in the solution was measured upon excitation at 488 nm and the emission was collected from 500 to 600 nm. The fluorescence intensity at 530 nm (Amax) was plotted against the irradiation time.

### Confocal Imaging.

HeLa cells were cultured in the 8 wells chamber at 37° C. After 80% confluence, the culture medium was removed and washed twice with 1×PBS buffer. Following incubation with the complexes formed from S-NPs and YOYO-1-DNA at the N/P ratio of 20 for 4 h, the medium was refreshed and cells were irradiated with white light (50 mW cm-2) for different time intervals. For some experiments, the cell nuclei were living stained with DRAQ5 following the standard protocols of the manufacturer (Biostatus). For S-NPs detection, the excitation was 405 nm, and the emission was collected above 560 nm; for YOYO-1 detection, the excitation was 488 nm, and the emission filter was 505-525 nm; for DRAQ5 detection, the excitation was 633 nm, and the emission was collected above 650 nm. For the lysosomal membrane damage study, HeLa cells were incubated with S-NPs and unlabeled DNA with exactly the same procedure as described above and stained with acridine orange (AO, 5 μM) for 10 min and then washed twice with 1×PBS. The cells were imaged immediately by confocal laser scanning microscope (CLSM, Zeiss LSM 410, Jena, Germany). The excitation was 488 nm, and the emission filter was 505-525 nm (green) and 610-640 nm (red). The images were analyzed by Image J 1.43×program (developed by NIH, http://rsbweb.nih.gov/ij/).

### DNA Transfection.

HeLa cells were seeded on 24-well plates at 5×104 cells per well and incubated for 24 h prior to transfection studies. The medium was then replaced by FBS-free DMEM medium, into which S-NPs complexed with eGFP-encoding plasmid DNA at 5 μg DNA mL-1 at an N/P ratio of 20 were added. For PEI25K/DNA complex, the N/P ratio is 10. After incubation for, 4 h, the medium was replaced by fresh one and cells were irradiated by white light (50 mW cm-2) for 5 min. Subsequently, cells were allowed to be cultured in fresh DMEM medium containing 10% FBS for another 44 h before assessment of GFP expression using flow cytometry (DakoCytomation) and CLSM. For flow cytometry, the mean fluorescence was determined by counting 10,000 events.

### Cytotoxicity Studies.

3-(4,5-Dimethythiazol-2-yl)-2,5-diphenyltetrazolium bromide (MTT) assays were used to assess the metabolic activity of HeLa cells. The cells were seeded in 96-well plates (Costar, IL, USA) at an intensity of 1×104 cells per well. After 24 h incubation, the medium was replaced with S-NPs/DNA complexes at an N/P ratio of 20 or PEI25K/DNA complexes at an N/P ratio of 10. Following incubation at 37° C. for 4 h, the cells were washed twice with 1×PBS and then exposed to light irradiation for 5 min at a power density of 50 mW cm-2. The cells were further incubated for 44 h and then washed twice with 1×PBS buffer, and 100 μL of freshly prepared MTT (0.5 mg mL-1) solution in culture medium was added into each well. The MTT medium solution was carefully removed after 3 h incubation in the incubator at 37° C. DMSO (100 μL) was then added into each well and the plate was gently shaken to dissolve all the precipitates formed. The absorbance of MTT at 570 nm was monitored by a microplate reader (Genios Tecan). Cell viability was expressed by the ratio of absorbance of the cells incubated with S-NPs/DNA to that of the cells incubated with culture medium only.

### Example 5

Light-Up Probes Based on a Fluorogen with AIE Characteristics for Live Cell and Nucleus Imaging and Targeted Cell Imaging

In another embodiment, the invention is an AIE fluorogen-based light-up probe for live cell imaging with nuclear targeting capability. Specifically, in an example embodiment, the present invention is an AIE probe able to selectively light-up HT-29 cells. As a proof of concept, the typical AIE fluorogen TPE is selected and functionalized with a water soluble cell-penetrating peptide with nuclear localization signal (NLS). Derived from trans-activator of transcription (TAT) viral proteins, the peptide sequence used Gly-Arg-Lys-Lys-Arg-Arg-Gln-Arg-Arg-Arg (SEQ ID NO: 5) is rich in positively charged arginine and lysine that facilitate cell uptake. The nuclear permeable AIE probe is water soluble and exhibits light-up response in nucleus through binding with nucleus components such as nucleic acids and nucleus proteins. In addition, a light-up probe for imaging of a specific type of cells was also demonstrated by conjugation with a cell targeting peptide.

### Click Synthesis of TPE-NLS Probe.

The azide-functionalized tetraphenylethene (TPE-N3) was prepared according to previous the report. TPE-N3 (3.5 mg, 9 μmol) and A-NLS (10 mg, 6.8 μmol) are dissolved in DMSO. Sodium ascorbate (0.7 mg, 3 μmol) and CuSO4 (0.3 mg, 1.5 μmol) dissolved in Milli-Q water are added into the DMSO mixture to initiate the click chemistry. The reaction is allowed to proceed at room temperature under shaking for ˜2 days. The product was obtained in ˜50% yield after HPLC purification. The final product is purified by preparative HPLC and characterized by LCMS-IT TOF and 1H NMR. IT-TOF-MS: m/z [M+3H]3+ calc. 622.037, found 622.038. 1H NMR (400 MHz, DMSO-d6, ppm) δ: 8.29 (b, 1H), 8.15 (b, 2H) 8.04-7.97 (m, 6H), 7.85 (s, 1H), 7.77-7.63 (m, 13H), 7.43 (s, 1H), 7.34 (s, 1H), 7.13-7.08 (m, 12H), 7.02-7.01 (m, 2H), 6.96-6.91 (m, 9H), 5.44 (s, 2H), 4.24-4.14 (m, 11H), 3.08-3.07 (m, 13H), 2.73 (b, 4H), 2.97 (m, 2H), 1.64-1.22 (m, 34H).

### Cell Culture.

MCF-7 breast cancer cells, U87MG brain tumor cells, and SKBR-3 cancer cells were cultured in DMEM containing 10% fetal bovine serum and 1% penicillin streptomycin at 37° C. in a humidified environment containing 5% CO2. Before experiment, the cells were pre-cultured until confluence was reached.

### Titration of TPE-NLS Probe Against Nucleus Components.

TPE-NLS DMSO stock solution is diluted with 1×PBS buffer in microplate wells. In each well varying amount of titrating agents, including as-hybridized double stranded DNA (dsDNA), histone and cell nucleus lysate are added into the solution. The final concentration of TPE-NLS is maintained as 10 μM. The fluorescence of the solution is recorded at excitation wavelength of 312 nm and emission wavelength of 480 nm.

### Cytotoxicity of TPE-NLS.

The metabolic activity of MCF-7 breast cancer cells was evaluated using methylthiazolyldiphenyltetrazolium bromide (MTT) assays. MCF-7 breast cancer cells were seeded in 96-well plates (Costar, IL, USA) at an intensity of 4×104 cells/mL, respectively. After 24 h incubation, the medium was replaced by TPE-NLS-contained FBS-Free medium at 50 μM, and the cells were then incubated for 4, 12 and 24 h, respectively. The wells were them washed twice with 1×PBS buffer and 100 μL of freshly prepared MTT (0.5 mg/mL) solution in culture medium was added into each well. The MTT medium solution was carefully removed after 3 h incubation in the incubator. Filtered DMSO (100 μL) was then added into each well and the plate was gently shaken for 10 min at room temperature to dissolve all the precipitates formed. The absorbance of MTT at 570 nm was monitored by the microplate reader (Genios Tecan). Cell viability was expressed by the ratio of the absorbance of the cells incubated with TPE-NLS to that of the cells incubated with culture medium only.

### Click Synthesis of TPE-GVH and TPE-D5G Probes.

Following a similar protocol for TPE-NLS, TPE-VHL and TPE-D5V probes were synthesized from TPE-N3 (2 mg, 5.2 μmol) and Alkyne-(Gly-Val-His-Leu-Gly-Tyr-Ala-Thr) (SEQ ID NO: 6) (6.9 mg, 7.8 μmol) or Alkyne-(Asp-Asp-Asp-Asp-Asp-Val-His-Leu-Gly-Tyr-Ala-Thr) (SEQ ID NO: 7) (11 mg, 7.8 μmol) via copper catalyzed click reaction, respectively. The reactions were allowed to proceed at room temperature under shaking for ˜2 days. The probe products TPE-GVH and TPE-DSG were obtained in ˜30% and ˜25% yield after HPLC purification. The final product were purified by preparative HPLC and characterized by HR-MS: m/z [M+2H]2+ calc. 909.8843, found 909.8805.

### Targeted Cell Imaging.

The HT-29, HeLa cancer cells and NIH-3T3 fibroblast cells were precultured in the chambers (LAB-TEK, Chambered Coverglass System) at 37° C. After 80% confluence, the medium was removed, and the adherent cells were washed twice with 1×phosphate buffered saline (PBS) buffer. The TPE-GVH or TPE-D5G probes in FBS-Free medium (1 μM) were then added to the chamber. After incubation for 4 h, respectively for these three cell lines, the cells were washed twice with 1×PBS buffer and used for confocal imaging. The fluorescence signal was collected between 430 and 605 nm upon excitation at 405 nm.

TPE-NLS is synthesized via click reaction between the azide-functionalized TPE and alkyne-bearing TAT NLS peptide (Alkyne-(Gly-Arg-Lys-Lys-Arg-Arg-Gln-Arg-Arg-Arg (SEQ ID NO: 5), A-NLS). The reaction takes place in a DMSO/water mixture under catalysis of sodium ascorbate and CuSO4 and the crude product is purified by HPLC. FIG. 11 illustrates the synthetic route for TPE-NLS.

The optical properties of TPE-NLS as well as its precursor TPE-N3 were studied by measuring their absorption and emission at the same concentration. Both TPE-NLS probe and TPE-N3 have absorption maxima in the region of 300-320 nm and emission maxima around 480 nm attributed to TPE moiety. The slight red shift in absorption maximum of TPE-N3 compared to TPE-NLS is due to the aggregate formation. As expected, TPE-NLS is virtually non-fluorescent in the DMSO/water mixture due to its good water solubility. In contrast, TPE-N3 emits strongly in the same solvent as their free molecular rotations are restricted in the aggregated state, which opens up radiative channels as the AIE phenomenon kicks in.

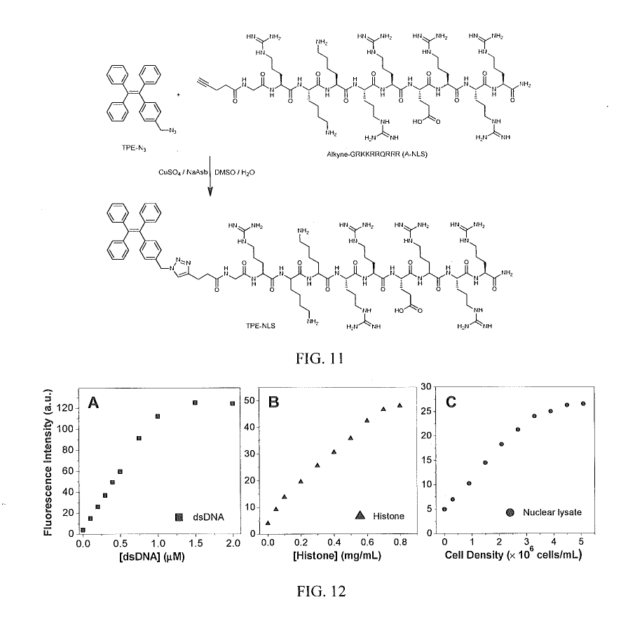

FIG. 12 illustrates the fluorescence intensity of 10 μM TPE-NLS upon addition of cellular components: dsDNA (A), histone (B) and nuclear lysate (C) at different concentrations in DMSO/1×PBS (1:99 v/v). λex=312 nm, λem=480 nm.

The addition of histone to TPE-NLS, on the other hand, also induces substantial fluorescence enhancement (FIG. 12B). As witnessed by the LLS measurements, the mean effective diameter of the probe/histone complex was found to be 238.7±38 nm, although a much weaker signal was obtained than that for dsDNA. As the protein has positive net charges (pKa ˜11.7) in PBS buffer, the interaction between histone and the probe is estimated to be mainly hydrophobic in nature. Therefore, in addition to the weak electrostatic interaction that causes aggregation of the TPE-NLS, the probe might also be engulfed by the hydrophobic pockets of the protein which restricted its molecular motion to activate AIE mechanism.

### Example 6

Light-Up Probe for Targeted and Activatable Photodynamic Therapy with Real-Time In-Situ Reporting of Sensitizer Activation and Therapeutic Responses

In another example embodiment, the present invention is a dual-targeted probe for real-time and in-situ self-reporting of photosensitizer activation and therapeutic responses. This probe allows multiplexed cellular imaging for traceable cancer cell ablation with single wavelength excitation. The probe can be cleaved by intracellular glutathione (GSH) to result in the red fluorescence turn-on for the PS activation monitoring and simultaneously release of the apoptosis sensor. The activated PS can generate ROS upon light irradiation to induce the cell apoptosis and activation of the caspase enzyme, which can be monitored by the AIEgen with green fluorescence turn-on.

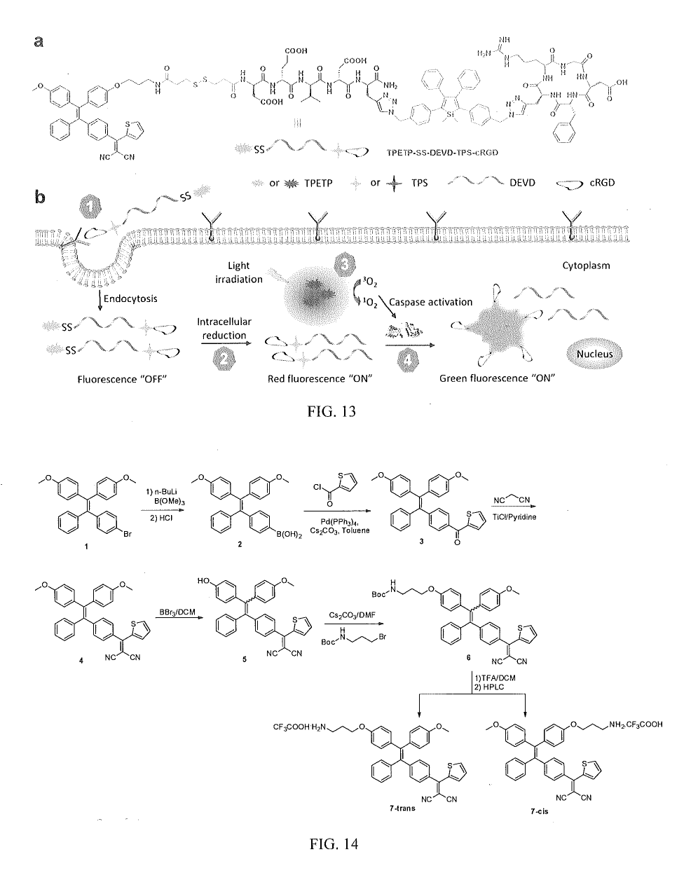

FIG. 13 is a schematic illustration of the dual-targeted theranostic probe. (a) The chemical structure of the probe. The probe was containing a photosensitizer/imaging agent with aggregation-induced emission (AIE) characteristic and a built-in light-up apoptosis sensor for noninvasive self-reporting of the photodynamic therapeutic responses in-situ. (b) The probe was non-fluorescent in aqueous media, but after uptake by cancer cells through receptor mediated endocytosis (1), the disulfate group can be cleaved through intracellular reduction by glutathione (GSH) to release the photosensitizer with red fluorescence turn-on as well as the apoptosis sensor which still maintained as off state (2). After light irradiation, the generated reactive oxygen species (ROS) can activate caspase enzymes (3), which can act on the apoptosis sensor to turn on the green fluorescence (4). The red fluorescence can be used for the image-guided photodynamic therapy while the green fluorescence can be used for the therapeutic response imaging.

Probe Design Principle.

It is known that AIE fluorogens highly emissive in aggregate state but their fluorescence is much weaker in molecularly dissolved state. It is rationalized that the propeller-shaped structure of AIE fluorogens and the free rotations of the phenyl rings can nonradiatively deactivated their excited states in molecularly dissolved state. However, the intramolecular rotations is restricted in the aggregates due to the physical constraint, which activates the radiative decay channel to result in fluorescence on. The fluorescence of AIEgens can be reduced after attaching with hydrophilic moiety which gives new possibilities of develop light-up probes without incorporating any quencher moieties. As shown in FIG. 13, the probe is composed of five components: (1) a dual functional red emissive tetraphenylethene (TPE) derivative with AIE characteristics to serve as an imaging agent and a PS; (2) a disulfate group that can be cleaved by high concentration of GSH in cancer cells; (3) a highly water soluble DEVD substrate that can be specifically cleaved by caspase-3/-7; (4) a green emissive AIE fluorogen for sensing caspase-3/-7 and (5) a hydrophilic cyclic arginine-glycine-aspartic acid (cRGD) for targeting cancer cells with overexpressed αᵥβ₃ integrin. The probe is water-soluble and shows very weak fluorescence in aqueous media due to the consumption of excitonic energy by the active intramolecular rotations. It is hypothesized, but not limited to the theory that, the probe can be selectively uptaken by αᵥβ₃ integrin overexpressed cancer cells through receptor mediated endocytosis and the AIE fluorogen with red fluorescence can be turn-on as an indication of PS activation due to the cleavage of the disulfate group by intracellular GSH and release the apoptosis sensor simultaneously. Upon light irradiation, the generated ROS can induce the cell apoptosis and activate the caspase-3/-7 which can cleave the DEVD substrate and lead to the green fluorescence of TPS. The green fluorescence turn-on can be used for real-time self-reporting of therapeutic response of photodynamic therapy.

Syntheses of TPETP-NH₂ and Identification of the Isomer.

Synthesis of the isomers is illustrated in FIG. 14. Compound 1 was treated with n-butyllithium, trimethyl borate and acid to yield compound 2 with a functional group of boronic acid, which underwent palladium-catalyzed coupling with acyl chlorides to yield compound 3. 3 was treated with TiCl₄ and malanonitirile to generate compound 4 with a dicyanovinyl group, which was subsequently treated with BBr₃ to generate 5 with one free hydroxyl group reaction between 5 and 3-(Boc-amino)propyl bromide yield compound 6. The compounds 1-6 were characterized by ¹H NMR, ¹³C NMR and mass spectroscopes. 6 was reacted with trifluoroacetic acid (TFA) to remove the Boc group to give the mixture of cis and trans isomers. The two isomers were separated with preparative high-performance liquid chromatography (HPLC) as red powders after freeze drying.

Syntheses of the Probe.

Bifunctionalized azide tetraphenylsilole (TPS-2N₃) was prepared according to methods known in the art. The double “click” reactions between TPS-2N₃ and alkyne-functionalized cRGD or DEVD were catalyzed by CuSO₄/sodium ascorbate in DMSO/water mixture (v/v=10/1), which afforded the apoptosis sensor DEVD-TPS-cRGD in 42% yield after HPLC purification. The purity and identity of DEVD-TPS-cRGD was well characterized by HPLC and mass characterization. Furthermore, the asymmetric functionalization of dithiobis(succinimidyl propionate) (DSP) with TPETP-NH₂ and amine-functionalized DEVD-TPS-cRGD in the presence of N, N-diisopropylethylamine (DIPEA) afforded the final probe TPETP-SS-DEVD-TPS-cRGD in 32% yield as red powders. The HPLC and mass characterization confirmed the right structure of the probe with high purity.

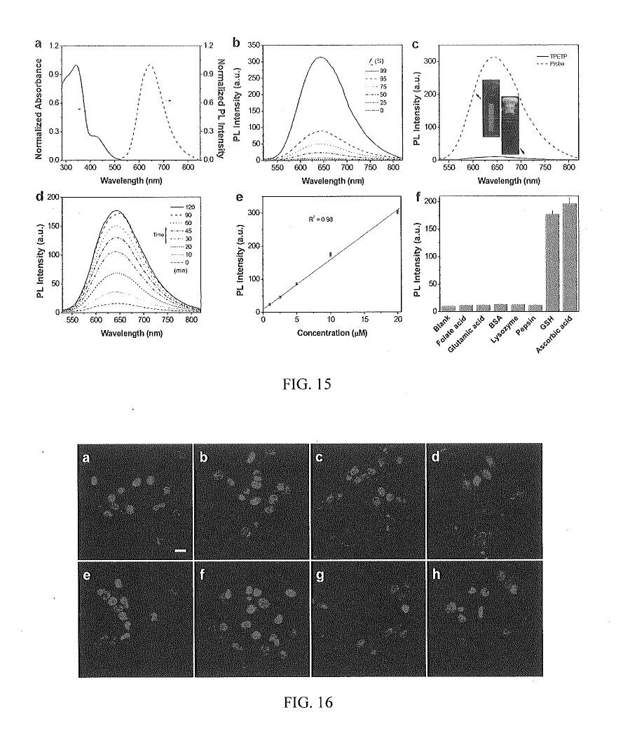

FIG. 15 illustrates the Reduction Responsiveness of the Probe.

(a) Normalized UV-vis absorption and PL spectra of TPETP in DMSO/water (v/v=1/199). (b) PL spectra of TPETP in DMSO/water mixtures at different water fractions (f_{w}). (c) PL spectra of TPETP and the probe in DMSO/PBS mixtures (v/v=1/199). Inset: the corresponding photographs taken under illumination of a UV lamp at 365 nm. (d) Time-dependent PL spectra of the probe (10 μM) incubated with GSH (0.1 mM). (e) Plot of PL intensity at 650 nm versus concentrations of the probe with the incubation of GSH (0.1 mM) for 75 min in DMSO/PBS (v/v=1/199). (f) Fluorescence response of the probe (10 μM) toward glutamic acid, folate acid, lysozyme, bovine serum albumin (BSA), pepsin, ascorbic acid or glutathione in DMSO/PBS (v/v=1/199). The excitation wavelength is 430 nm. Data represent mean values±standard deviation, n=3.

Prototypical Properties of the Probe and Activation by GSH.

The UV-vis absorption and photoluminescence (PL) spectra of TPETP in DMSO/PBS (v/v=1/199) buffer are shown in FIG. 15a. The UV-vis absorption of TPETP is in the range of 400-500 nm with an absorption maxima at 430 nm. The emission spectrum is ranged from 550 nm to 850 nm with the maximum at 640 nm. To study whether the TPETP retains its AIE properties, the PL intensities of TPETP in DMSO and water mixtures with different water fractions (f_{w}) were studied. As shown in FIG. 15b, TPETP is almost non-fluorescent in DMSO solution (f_{w}=0) which should be due to the free rotation of the TPE phenyl rings in molecularly dissolved state. However, the fluorescence intensity of TPETP increased steadily when the f_{w} is increased. The fluorescence intensity of TPETP at f_{w} of 99% is 120-fold higher than that in DMSO. This fluorescence intensity increase with the f_{w} increase is due to that TPETP molecules tend to aggregate in poor solvents and result in the restriction of the intramolecular motion. The results above clearly demonstrated that TPETP retains its AIE characteristic.

After attaching hydrophilic peptides, the probe TPETP-SS-DEVD-TPS-cRGD is almost non-fluorescent in DMSO/PBS (v/v=1/199). In contrast, TPETP shows intense red fluorescence in the same mixture solvent. The significant difference in the PL intensities of the disulfate group containing probe and TPETP offers good opportunity for the development of cancer cell specific light-up probe due to the elevated concentration of GSH compared to normal cells. The response of the probe to GSH was studied by monitoring the fluorescence intensity change of the probe incubated with GSH over time in DMSO/PBS (v/v=1/199). A quick and steady red fluorescence increase is observed over time after the addition of GSH to the probe solution. The fluorescence intensity reaches a plateau after 90 min incubation which is 14-fold higher than the intrinsic fluorescence intensity of the probe itself. The gradual red fluorescence intensity increase after incubating with GSH should be due to the increased amount of cleaved TPETP residues and forms aggregates in aqueous media to lead to red fluorescence turn-on. The molecular dissolution of the probe and the aggregation of the TPETP residue were confirmed by laser light scattering (LLS) measurements. No LLS signals could be detected from the probe while the TPETP residue after the GSH treatment tends to aggregate with an average diameter of 148±12.2 nm. The aggregation formation clearly explains the probe fluorescence turn-on after incubation with GSH. Subsequently, the probe at different concentrations were incubated with GSH for 90 min and the corresponding fluorescence change were recorded. The probe selectivity studies show that the fluorescence was only increased in the presence of the reducing agent while the probe incubated with other bio-acids and proteins showed negligible fluorescence change. These results indicate that the red fluorescence turn-on is attributed to the reduction of the disulfate group of the probe with the release of the TPETP residue upon encountering with reducing agent such as GSH or ascorbic acid.

The generation of ROS upon light irradiation of the PS is the key step for efficient photodynamic therapy. The ROS generation of the released TPETP residue was studied by measuring the absorption decrease of the mixture of the probe and the ROS indicator 9,10-anthracenediyl-bis(methylene)dimalonic acid (ABDA) in DMSO/PBS (v/v=1/199) upon light irradiation. It should be noted that the absorbance of the probe does not contribute to the measured absorbance change due to its very low concentration. The absorption peaks at 358 nm, 378 nm and 400 nm attributed to the anthracene moiety in ABDA decreased gradually upon light irradiation, as a result of fast reaction between ROS and the anthracene moiety. With light irradiation of the solution for 12 min, the absorption at 400 nm is decreased from 100% to 22.4% of its original value, indicative of efficient ROS generation. However, when vitamin C (VC, a well-known ROS scavenger) was added, the absorbance decrease was remarkably inhibited (from 100% to 93.8% of its original value after 12 min of light irradiation), further confirming the ROS generation.

Caspase-3/-7 activation of the released apoptosis sensor. The absorption maximum of TPS is 365 nm and the emission maximum is 480 nm. TPS also shows the AIE characteristic, which was demonstrated by the PL intensity of TPS in different fw in DMSO/water mixture. Both the GSH-pretreated probe and the apoptosis sensor DEVD-TPS-cRGD shows limited green fluorescence as compared to TPS at the same concentrations in DMSO/PBS (v/v=1/199). These results indicate that the release of the apoptosis sensor activated by GSH will not yield obvious fluorescence of TPS. However, fluorescence intensity increase of TPS is recorded for GSH-pretreated probe (10 μM) upon further treatment with recombinant human caspase-3/-7. As caspase-3/-7 can specifically cleave the DEVD substrate, which leads to the release of hydrophobic TPS residues with green fluorescence turn-on. The TPS fluorescence intensity reaches a plateau after 60 min treatment of caspase-3 (100 pM), which is 18-fold higher than the intrinsic emission of the GSH-pretreated probe. However, the fluorescence intensity change is prohibited in the presence of 5-[(S)-(+)-2-(methoxymethyl)pyrrolidino]sulfonylisatin, a highly specific inhibitor of caspase-3/-7,37 confirming that the TPS fluorescence increase is due to the specific cleavage of DEVD substrate. The aggregation formation of the cleaved TPS residue of the apoptosis sensor was studied by LLS, which showed an average diameter of 134±14.6 nm. The caspase-3 concentration dependent TPS fluorescence change was further monitored to check whether it is possible to quantify the caspase concentration through fluorescence intensity change

The selectivity of the apoptosis sensor was studied by incubating the GSH-pretreated probe with lysozyme, pepsin and bovine serum albumin (BSA) and other caspase enzymes. Only caspase-3/-7 treated groups display fluorescence intensity increase, confirming that DEVD substrate is specifically cleaved by caspase-3/-7. As there are many kinds of enzymes exist in the cells, we further incubated the probe with cellular lysate of normal and apoptotic MDA-MB-231 cancer cells, which were obtained by treating the cells with staurosporine (STS, 2 μM), a commonly used cell apoptosis inducer, to activate the caspase-3/-7 enzyme.38 The cell lysates of normal and apoptotic cells were directly incubated with the probe (10 μM) and the fluorescence intensity at 640 and 480 nm was monitored over time. The fluorescence intensity at 640 increases quickly in both the normal cells and the apoptotic cells. However, the fluorescence at 480 nm only showed fluorescence increase in apoptotic cells while a minimum fluorescence changed in normal cell lysate. These results indicate that the red fluorescence of TPETP can be activated by normal and apoptotic cells while the green fluorescence of TPS can only be activated in apoptotic cells.

The PL spectra of TPETP and the probe in DMSO and phosphate buffered saline (PBS, pH=7.4) mixtures (v/v=1/199) are shown in FIG. 15c. After attaching hydrophilic peptides, the probe TPETP-SS-DEVD-TPS-cRGD is almost non-fluorescent in DMSO/PBS (v/v=1/199). In contrast, TPETP shows intense red fluorescence in the same mixture solvent. The significant difference in the PL intensities of the disulfate group containing probe and TPETP offers good opportunity for the development of cancer cell specific light-up probe due to the elevated concentration of GSH compared to normal cells.

Intracellular Red Fluorescence Turn-on.

To demonstrate the specific αᵥβ₃ integrin overexpressed cancer cell light-up imaging, the probe was incubated with αᵥβ₃ integrin overexpressed MDA-MB-231 breast cancer cells and low αᵥβ₃ integrin expressed MCF-7 breast cancer cells as well as 293T normal cells as the negative control. As shown in FIGS. 16A-H, the red fluorescence which should be attributed to the released TPETP residues in MDA-MB-231 cells upon incubation with the probe intensifies gradually with the increase of incubation time. In addition, the red fluorescence signal in MDA-MB-231 is much stronger than those in MCF-7 and 293T cells under the identical experimental conditions, which should be due to the lower densities of receptors on the cell surface of the later. This was further confirmed by pre-treatment of the MDA-MB-231 cells with free cRGD prior to the probe incubation which also shows dramatically reduced fluorescence intensity. The release of the TPETP residues was confirmed by the pretreatment of the MDA-MB-231 cells with an inhibitor of g-glutamylcysteine synthetase buthionine sulfoximine (BSO) to inhibit the cells from synthesizing GSH, which also shows very weak red fluorescence intensity. These specific red fluorescence light-up in cells were also confirmed by flow cytometry analysis and the results am in well accordance with the confocal images. These results clearly demonstrate that the probe can be specifically uptake by MDA-MB-231 cells through receptor mediated endocytosis and the red fluorescence can be turn-on in the presence of intracellular GSH, which can be used for the monitoring of the PS activation and the specific imaging of cancer cells.

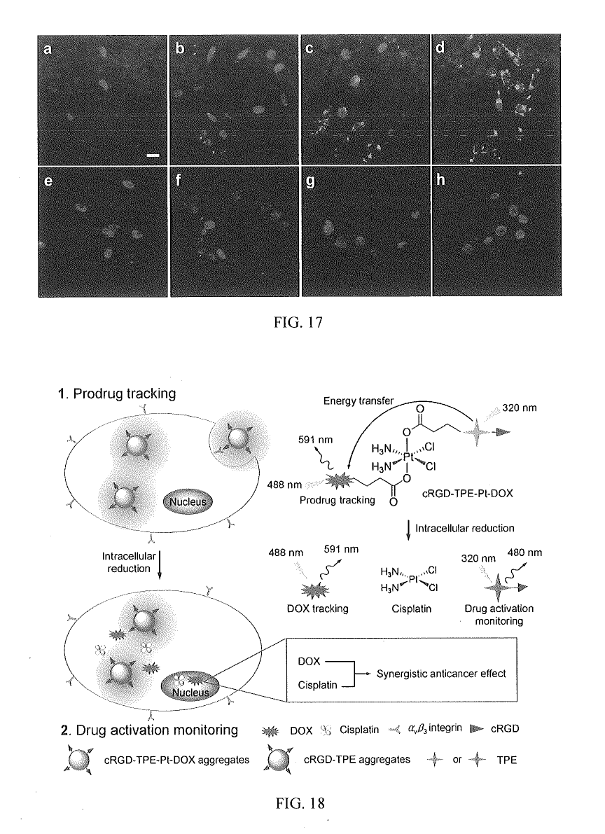

FIGS. 17A-H illustrate the real-time cell apoptosis imaging. Confocal images of MDA-MB-231 cells (a-f), MCF-7 cells (g), 293T cells (h) or MDA-MB-231 cells treated with cRGD (e) or VC (f) and incubated with the probe for 4 h with light irradiation of 1 min (a), 2 min (b), 4 min (c), 6 min (d-h). The blue fluorescence from the nuclei of cells were living stained with Hoechst (Eₓ: 405 nm; Eₘ: 430-470 nm); the green fluorescence is from the TPS (Eₓ: 405 nm; Eₘ: 505-525 nm). All images share the same scale bar (20 μm).

Synthesis of amine functionalized DEVD-TPS-cRGD through “click” reactions. TPS-2N₃ (10.0 mg, 19.2 μmol), alkyne-functionalized cRGD (10.8 mg, 19.2 μmol) and alkyne-functionalized DEVD (10.8 mg, 19.2 μmol) were dissolved in a mixture of DMSO/H₂O solution (v/v=10/1, 2.0 mL). Then CuSO₄ (9.4 mg, 38.4 μmol) and sodium ascorbate (15.2 mg, 38.4 μmol) were sequential added to the above mixture solution. The reaction was continued with stirring overlight. The final product was obtained after purification using preparative HPLC and lyophilized under vacuum to yield the amine functionalized DEVD-TPS-cRGD as white powders in 41% yield (13.1 mg). HPLC (λ=320 nm): purity 98.6%, retention time 11.2 minutes. ESI-MS: m/z [M+H]⁺ calc. 1665.845, found 1665.046.

Synthesis of the Probe TPETP-SS-DEVD-TPS-cRGD.

Detailed description of the synthesis and characterization of TPETP-NH₂ can be found in the Supplementary Methods. Amine terminated DEVD-TPS-cRGD (10.0 mg, 6.0 μmol) and TPETP-NH₂ (3.6 mg, 6.0 μmol) were dissolved in anhydrous DMSO (1.0 mL) in the presence of DIPEA (1.0 μL). The mixture was stirred for 10 min at room temperature. Then dithiobis(succinimidyl propionate) (DSP, 2.4 mg, 6.0 μmol) in DMSO (0.5 mL) was added quickly to the above solution. The reaction was continued with stirring at room temperature for another 24 h. The final product was obtained after purification using preparative HPLC and lyophilized under vacuum to yield the probe TPETP-SS-DEVD-TPS-cRGD as yellow powders in 32% yield (4.7 mg). HPLC (λ=320 nm): purity 97.3%, retention time 12.3 minutes; ESI-MS: m/z [M+2H]²⁺ calc. 1216.945, found 1215.916.

Referring again to FIG. 14, the following synthetic methods are described.

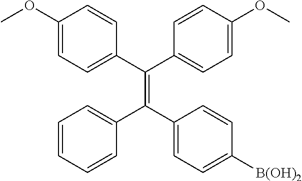

To the solution of compound 1 (7.7 g, 16.3 mmol) in THF (150 mL) was added n-butyllithium (1.6 M in hexane, 16.0 mL) at −78° C. The mixture was stirred at the same temperature for 2 h. Then trimethyl borate (3.8 mL, 33.4 mmol) was added. The reaction mixture was then allowed to warm up and stirred at room temperature for 3 h. The reaction was quenched by addition of HCl solution (3 M, 45 mL) and the resulting solution was stirred at room temperature for 5 h. Then the mixture was diluted with ethyl acetate (100 mL) and brine (200 mL). The organic phase was separated, washed with brine (100 mL×2), and dried over MgSO4. The mixture was filtered and the filtrate was concentrated under reduced pressure. The desired residue was subjected to flash chromatography (hexane/ethyl acetate=10/1-2/1) to yield compound 2 as white solid (2.9 g, 40.8% yield), which was used directly in the next step without further purification.

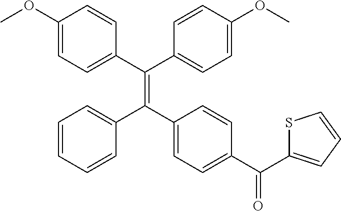 [SMILES: COc1ccc(C(=C(c2ccccc2)c2ccc(C(=O)c3cccs3)cc2)c2ccc(OC)cc2)cc1]

To the suspension of compound 2 (2.9 g, 6.5 mmol) in toluene (80 mL) was added anhydrous cesium carbonate (5.3 g, 16.2 mmol) and tetrakis(triphenylphosphine) palladium(0) (228 mg, 0.32 mmol). Thiophene-2-carbonyl chloride (2.0 g, 13.6 mmol) was added to the above mixture. Then the mixture was stirred at 100° C. for 12 h. After it was cooled down to room temperature, the mixture was washed with water (50 mL) and brine (50 mL). The organic layer was dried over MgSO₄, filtered and filtrate was concentrated and purified by chromatography (hexane/ethyl acetate=50/1-10/1) to give the desired product as orange solid (2.8 g, 85.8% yield). 1H NMR (400 MHz, CDCl3) δ 7.68 (dd, J1=1.2 Hz, J2=4.8 Hz, 1H), 7.64 (m, 2H), 7.60 (dd, J1=1.2 Hz, J2=4.0 Hz, 1H), 7.11-7.15 (m, 6H), 7.05 (m, 2H), 6.94-6.97 (m, 4H), 6.63-6.67 (m, 4H), 3.75 (s, 3H), 3.74 (s, 3H); 13C NMR (100 MHz, CDCl3) δ 187.0, 158.4, 158.3, 143.7, 143.6, 141.8, 138.1, 135.8, 135.7, 135.4, 1343, 133.7, 132.6, 132.5, 131.4, 131.3, 128.8, 127.8, 127.7, 126.4, 113.2, 113.0, 55.1, 55.0; HRMS (EI) calcd for [M]+: 502.1603, found: 502.1605.

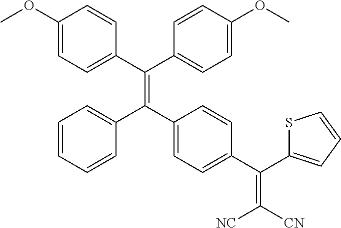 [SMILES: COc1ccc(C(=C(c2ccccc2)c2ccc(C(=C(C#N)C#N)c3cccs3)cc2)c2ccc(OC)cc2)cc1]

To the solution of compound 3 (0.26 g, 0.52 mmol) and malononitrile (45 mg, 0.68 mmol) in dichloromethane (10 mL) was added titanium tetrachloride (0.20 mL, 1.8 mmol) slowly at 0° C. After the reaction mixture was stirred for 30 min, pyridine (0.15 mL, 1.8 mmol) was injected and stirred for another 30 min. Then the mixture was heated at 40° C. for 4 h. After the mixture was cooled down to room temperature, the reaction was quenched by water (10 mL) and the mixture was extracted with dichloromethane. The collected organic layer was washed by brine (20 mL), dried over MgSO₄ and concentrated under reduced pressure. The desired residue was purified by column chromatography (hexane/ethyl acetate=50/1-10/1) to give the desired product as red solid (230 mg, 81.0% yield). 1H NMR (400 MHz, CDCl3) δ 7.80 (dd, J1=1.2 Hz, J2=5.2 Hz, 1H), 7.73 (dd, J1=1.2 Hz, J2=5.2 Hz, 1H), 7.13-7.22 (m, 8H), 7.06 (m, 2H), 8.91-8.98 (m, 4H), 8.64-8.68 (m, 4H), 3.75 (s, 6H); 13C NMR (100 MHz, CDCl3) δ 164.8, 158.6, 158.4, 148.7, 143.2, 142.4, 1383, 137.7, 136.1, 135.7, 135.5, 133.5, 132.6, 132.5, 131.5, 131.3, 129.1, 128.8, 127.9, 126.5, 114.5, 113.8, 113.2, 113.0, 55.1, 55.0. MS (EI) calcd for [M]+: 550.1709, found: 550.1708.

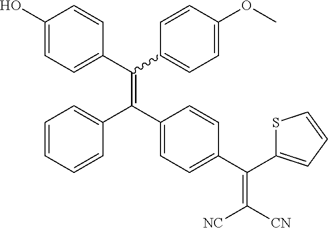 [SMILES: COc1ccc(C(=C(c2ccccc2)c2ccc(C(=C(C#N)C#N)c3cccs3)cc2)c2ccc(O)cc2)cc1]

To the solution of compound 4 (170 mg, 0.31 mmol) in dichloromethane (10 mL) was added boron tribromide (1.0 M in dichloromethane, 0.50 mmol) at 0° C. Then the reaction mixture was stirred at room temperature for 3 h. The reaction was quenched by addition of water (5 mL) under ice-water bath. The organic layer was taken, washed with brine (15 mL), dried over MgSO₄ and concentrated under reduced pressure. The desired residue was purified by column chromatography (hexane/ethyl acetate=20/1-5/1) to give the desired product as red solid (43 mg, 25.8% yield). 1H NMR (400 MHz, CDCl3) δ 7.79-7.81 (m, 1H), 7.71-7.73 (m, 1H), 7.11-7.22 (m, 8H), 7.06-7.08 (m, 2H), 6.90-6.99 (m, 4H), 6.64-6.68 (m, 2H), 6.57-6.61 (m, 2H), 3.75 (s, 3H); 13C NMR (100 MHz, CDCl3) δ 165.2, 164.9, 158.5, 158.4, 154.9, 154.7, 148.9, 148.7, 143.1, 142.5, 142.3, 138.7, 138.6, 137.7, 137.6, 136.4, 136.2, 136.1, 135.9, 135.5, 135.4, 135.3, 133.5, 133.4, 132.8, 132.7, 132.6, 132.5, 131.5, 131.3, 129.1, 128.9, 128.8, 127.9, 126.5, 114.9, 114.6, 114.4, 11.3.8, 113.7, 113.2, 113.0, 55.1, 55.0; HRMS (EI) calcd for [M]+: 536.1658, found: 536.1654.

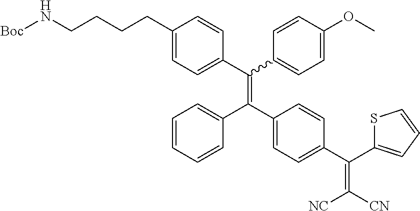 [SMILES: COc1ccc(C(=C(c2ccccc2)c2ccc(C(=C(C#N)C#N)c3cccs3)cc2)c2ccc(CCCCNC(=O)OC(C)(C)C)cc2)cc1]

To the solution of compound 5 (40 mg, 0.075 mmol) in DMF (5 mL) was added 3-(Boc-amino)propyl bromide (35 mg, 0.15 mmol) and caesium carbonate (50 mg, 0.15 mmol). The mixture was stirred at room temperature for 6 h. Then ethyl acetate (50 mL) and brine (50 mL) were added to the reaction mixture. The organic layer was taken, washed with brine (50 mL×4), dried over MgSO₄ and concentrated under reduced pressure. The desired residue was purified by column chromatography (hexane/ethyl acetate=30/1-8/1) to give the desired product as red solid (28 mg, 53.3% yield). 1H NMR (400 MHz, CDCl3) δ 7.81 (dd, J1=1.2 Hz, J2=5.2 Hz, 0.5H), 7.80 (dd, J1=1.2 Hz, J2=5.2 Hz, 0.5H), 7.71-7.73 (m, 1H), 7.12-7.22 (m, 8H), 7.05-7.07 (m, 2H), 6.89-6.98 (m, 4H), 6.62-6.67 (m, 4H), 3.96 (t, d=6.0 Hz, 2H), 3.74 (s, 3H), 3.32 (m, 2H), 1.94 (m, 2H), 1.44 (s, 4.5H), 1.43 (s, 4.5H); HRMS (ESI) calcd for [M+H]+: 694.2734, found: 694.2731.

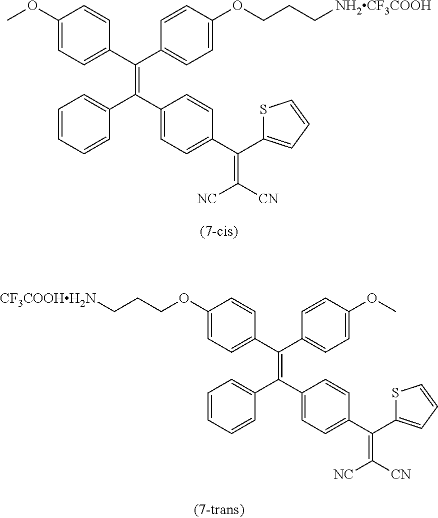

To the solution of compound 6 (28 mg, 0.04 mmol) in dichloromethane (5 mL) was added trifluoroacetic acid (1 mL). The resulting mixture was stirred at room temperature for 6 h. Then the mixture was concentrated under reduced pressure. The desired oil was separated by high performance liquid chromatography (HPLC) using acetonitrile and water as gradient elution buffer to give 7-cis (10.0 mg as red solid, 43.4% yield): 1H NMR (500 MHz, CDCl3) δ 8.27 (dd, J1=1.0 Hz, J2=5.0 Hz, 1H), 7.77 (brs, 3H), 7.67 (dd, J1=1.5 Hz, J2=4.0 Hz, 1H), 7.38 (dd, J1=4.0 Hz, J2=5.0 Hz, 1H), 7.35 (m, 2H), 7.17-7.20 (m, 2H), 7.10-7.14 (m, 3H), 7.01-7.03 (m, 2H), 6.84-6.90 (m, 4H), 6.68-6.72 (m, 4H), 3.96 (t, J=6.0 Hz, 2H), 3.68 (s; 3H), 2.95 (m, 2H), 1.98 (m, 2H); HRMS (ESI) calcd for [M+H]+: 594.2210, found: 594.2215; 7-trans (8.0 mg as red solid, 34.8% yield): 1H NMR (500 MHz, CDCl3) δ 8.27 (dd, J1=1.0 Hz, J2=5.0 Hz, 1H), 7.73 (brs, 3H), 7.66 (dd, J1=1.0 Hz, J2=4.0 Hz, 1H), 7.38 (dd, J1=4.0 Hz, J2=5.0 Hz, 1H), 7.35 (m, 2H), 7.18-7.21 (m, 2H), 7.09-7.15 (m, 3H), 7.02-7.04 (m, 2H), 6.92 (m, 2H), 6.85 (m, 2H), 6.68-6.73 (m, 4H), 3.98 (t, J=6.0 Hz, 2H), 3.67 (s, 3H), 2.96 (m, 2H), 1.98 (m, 2H); HRMS (ESI) calcd for [M+H]+: 594.2210, found: 594.2212.

### Example 7

### Aggregation-Induced Emission Fluorogens for Drug Tracking and Delivery

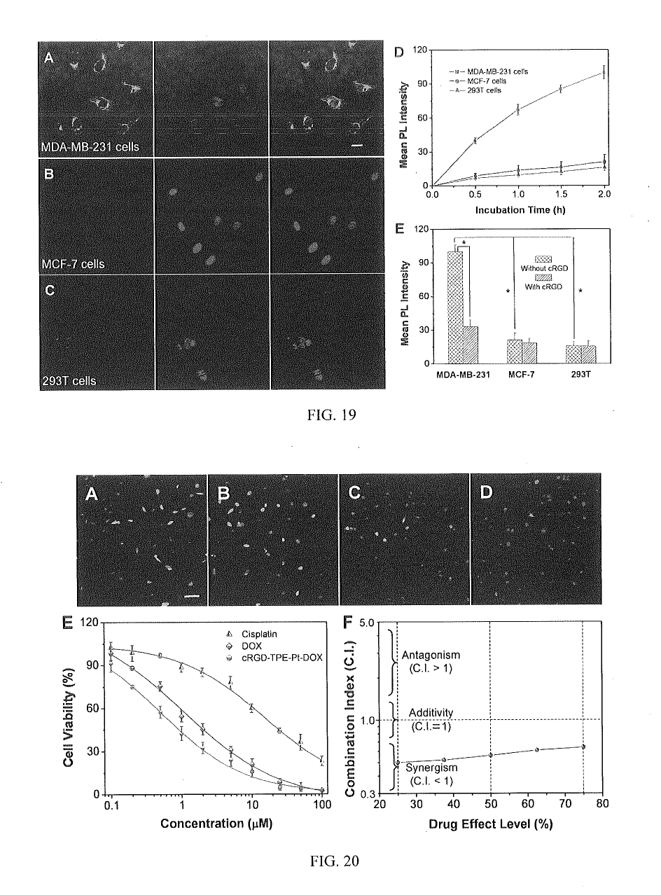

In another example embodiment, the present invention is a simple targeted theranostic delivery system containing two prodrugs which can be utilized for prodrug tracking, dual-drug activation monitoring with reduced side effects and enhanced therapeutic efficiency. The prodrug is composed of a targeted cRGD moiety, a luminogen tetraphenylene (TPE) with AIE characteristics as an energy donor, and a fluorescent anticancer drug doxorubicin (DOX) as an energy receptor using a chemotherapeutic Pt(IV) prodrug as the linker. The prodrug can accumulate preferentially in cancer cells with overexpressed αvβ3 integrin and release the active drug Pt(II) (cisplatin) and DOX simultaneously for their respective biological actions upon intracellular reduction. FIG. 18 illustrates the targeted dual-acting prodrug for real-time drug tracking and activation monitoring.

Difunctionalized azide tetraphenylethene was firstly synthesized according to methods known by those of skill in the art. Two consecutive “click” reactions of TPE-2N3 with alkyne-functionalized cRGD and propargylamine using CuSO4/sodium ascorbate as the catalyst in DMSO/water (v/v=1/1) afforded cRGD-TPE in 53% yield after HPLC purification.

Commercially available anticancer drug cisplatin was modified to be used as the linker between cRGD-TPE and doxorubicin (DOX). N-Hydroxysuccinimide (NHS) activated cis, cis, transdiamminedichlorodisuccinatoplatinum(IV) complex (NHS-Pt-NHS) as the linker was prepared. Asymmetric functionalization of the activated Pt(IV) linker with cRGD-TPE and DOX in the presence of N, N-diisopropylethylamine (DIPEA) in anhydrous DMSO afforded cRGD-TPE-Pt-DOX in 36% yield after HPLC purification.

Cancer-targeted drug delivery can increase the drug accumulation in targeted tissues. To demonstrate the feasibility of achieving cancer-targeted delivery of the prodrug, cRGD-TPE-Pt-DOX was incubated with MDA-MB-231, MCF-7 breast cancer cells and normal 293T cells. MDA-MB-231 cells with overexpressed integrin αvβ3 on cellular membrane were chosen as integrin-positive cancer cells, while MCF-7 and 293T cells with low αvβ3 integrin expression were used as the negative controls. The confocal imaging results are shown in FIG. 19. After 2 h incubation, strong fluorescence (colored as green) from cRGD-TPE-Pt-DOX in cytoplasm is observed in MDA-MB-231 cells (FIG. 19A), which is much brighter than those in MCF-7 (FIG. 19B) and 293T cells (FIG. 19C). Semi-quantitative fluorescence intensity analysis of cRGD-TPEPt-DOX in these cells was monitored at different incubation time points (FIG. 19D).

FIGS. 19A-E is an evaluation of the targeting effect of cRGD-TPE-Pt-DOX to different cells: confocal images of MDA-MB-231 (A), MCF-7 (B) cancer cells and 293T (C) normal cells after incubation with cRGD-TPE-Pt-DOX for 2 h. The masked green color represents fluorescence from cRGD-TPE-Pt-DOX (λex=488 nm) and the red color represents fluorescence from the nuclei of cells stained by DRAQ5. All images share the same scale bar (20 μm). (D) Relative fluorescence intensity of cRGD-TPE-Pt-DOX (λex=488 nm) determined in MDA-MB-231, MCF-7 and 293T cells at different incubation time. (E) Relative fluorescence intensity of cRGD-TPE-Pt-DOX determined in MDA-MB-231, MCF-7 and 293T cells with and without cRGD (50 μM) pretreatment. The error is the standard deviation from the mean (n=3, * is P<0.05).

Subsequently, cRGD-TPE, free DOX and cRGD-TPE-Pt-DOX were incubated with MDAMB-231 breast cancer cells and the drug activation was studied by CLSM. As shown in FIG. 20A, both free DOX and the succinic acid modified DOX (“green” color) can quickly diffuse to the cell nucleus where its anti-cancer functions are executed after 6 h incubation, which has good coincidence with DRAQ5 stained nucleus (“red” color). The cRGD-TPE-Pt-DOX upon incubation with MDA-MB-231 cells was studied and the CLSM images at different incubation time were collected. As shown in FIG. 20C, after 1 h incubation, the DOX fluorescence (“green”) can be clearly observed, which indicates efficient cellular uptake of the prodrug. Meanwhile, a weak “blue” fluorescence of TPE is detected due to the initial drug activation in the cells.

FIG. 20 illustrates CLSM images of MDA-MB-231 cells after incubation with cRGD-TPE (A), cisplatin (B), DOX (C), and cRGD-TPE-Pt-DOX (D) for 72 h. Viable cells were stained green with calcein-AM, and dead cells were stained red with PI. All images share the same scale bar (50 μm). (E) Dose-effect profiles for MDA-MB-231 breast cancer cells after incubation with cisplatin, DOX, and cRGD-TPE-Pt-DOX for 72 h. (F) Combination index (CI) plots for cRGD-TPE-Pt-DOX against MDA-MB-231 cells at different drug effect levels.

The toxicity of cRGD-TPE-Pt-DOX to different cells was also studied using MDA-MB-231, MCF-7 and 293T cells as examples. After incubation with cRGD-TPE-Pt-DOX for 6 h, the cells were stained with Annexin V-FITC/Propidium Iodide (PI), which are commonly used fluorescent probes to distinguish viable cells from apoptosis ones. Only MDA-MB-231 cells show strong apoptotic fluorescence, and the fluorescence from MCF-7 and 293T cells is negligible, which indicates that cRGD-TPE-Pt-DOX is able to selectively kill integrin overexpressed cancer cells. This should be due to the integrin mediated endocytosis, which leads to selective cellular uptake of the prodrug cRGD-TPE-Pt-DOX.

To confirm the drug synergy in cRGD-TPE-Pt-DOX, the combination index (C.I.) was calculated. The C.I. derived from the dose-effect profiles was plotted against drug effect level, which provided quantitative information of the combination drug effect, where C.I. values lower than, equal to, or higher than 1 denote synergism, additivity, or antagonism, respectively. As shown in FIG. 20F, C.I. plots for cRGD-TPE-Pt-DOX clearly demonstrate synergistic effect against MDA-MB-231 cells over a wide range of drug effect levels from 75% to 25%. These results prove that the delivery of cisplatin and DOX in the form of prodrug in cRGD-TPE-Pt-DOX has resulted in enhanced cancer cell killing effect.

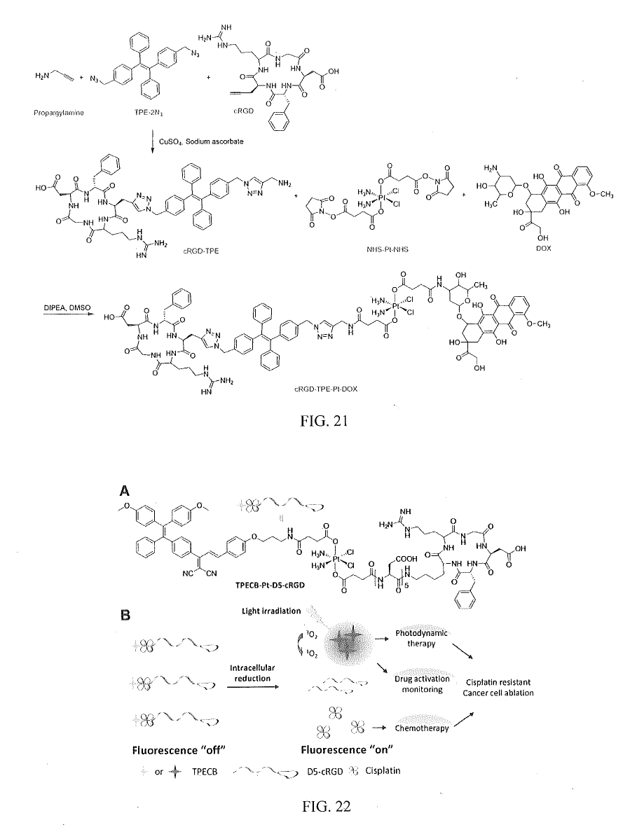

The synthetic route of the compounds described above is illustrated in FIG. 21.

Synthesis of Amine Functionalized cRGD-TPE Through Two Consecutive “Click” Reactions.

TPE-2N3 (15 mg, 34 μmol) and alkyne-functionalized cRGD (19.4 mg, 34 μmol) were dissolved in a mixture of DMSO/H2O solution (v/v=1/1, 2.0 mL). The “click” reaction was initiated by sequential addition of CuSO4 (19.2 mg, 12 μmol) and sodium ascorbate (4.8 mg, 24 μmol). The reaction was continued with shaking at room temperature for 12 h. Then propargylamine (4.4 μL, 68 μmol), CuSO4 (19.2 mg, 12 μmol), sodium ascorbate (4.8 mg, 24 μmol) was added sequentially and reacted at room temperature for another 24 h. The final product was purified by preparative HPLC and lyophilized under vacuum to yield the amine functionalized cRGD-TPE as white powders in 53% yield (19.2 mg). HPLC (λ=320 nm): purity 98.6%, retention time 10.3 minutes. ¹H NMR (DMSO-d6, 400 MHz), δ (TMS, ppm): 12.24 (s, 1H), 8.22 (m, 3H), 8.01 (m, 2H), 7.78 (s, 2H), 7.10 (m, 11H), 6.94 (m, 12H), 5.43 (m, 4H), 4.62 (t, 1H), 4.41 (m, 2H), 4.10 (m, 2H), 3.13 (m, 4H), 2.90 (m, 3H), 2.65 (m, 2H), 2.38-2.27 (m, 2H), 1.75 (m, 1H), 1.46 (m, 2H), 1.35 (m, 2H). ESI-MS: m/z [M+H]+ calc. 1068.495, found 1068.806.

Synthesis of Theranostic Dual-Acting Prodrug cRGD-TPE-Pt-DOX

Amine terminated cRGD-TPE (10.7 mg, 10 μmol) and doxorubicin hydrochloride (5.8 mg, 10 μmol) were dissolved in anhydrous DMSO (1.0 mL) with a catalytic amount of DIPEA (1.0 μL). The mixture was stirred at room temperature for 10 min. Then N-hydroxysuccinimide-activated platinum(IV) complex (7.3 mg, 10 μmol) in DMSO (0.5 mL) was added quickly to the above mixture. The reaction was continued with stirring at room temperature for another 24 h. The final product was purified by preparative HPLC and lyophilized under vacuum to yield the prodrug cRGD-TPE-Pt-DOX as red powders in 36% yield (7.6 mg). HPLC=320 nm): purity 97.3%, retention time 17.2 minutes. ¹H NMR (DMSO-d6, 400 MHz): 12.24 (s, 1H), 8.38 (t, 1H), 8.24 (m, 3H), 8.08-7.88 (m, 4H), 7.72 (m, 1H), 7.57 (d, 1H), 7.20-6.98 (m, 12H), 6.96-6.79 (m, 12H), 6.46 (m, 6H), 5.43 (m, 4H), 5.24 (s, 1H), 4.93 (m, 1H), 4.57-4.68 (m, 2H), 4.30-4.51 (m, 4H), 4.15-4.07 (m, 1H), 4.07 (m, 2H), 3.97 (s, 3H), 3.79 (m, 1H), 3.62-3.53 (m, 3H), 3.15 (m, 2H), 2.95 (m, 2H), 2.84 (m, 2H), 2.65 (m, 3H), 2.12 (m, 2H), 2.35-2.27 (m, 2H), 1.83 (d, 1H), 1.77-1.65 (m, 3H), 1.60-1.36 (m, 4H), 1.13 (m, 3H); ESI-MS: m/z [M+H]+ calc. 2109.642, found 2109.698.

### Determination of Combination Index (C.I).

The combination therapy of cisplatin and DOX towards MDA-MB-231 cells was evaluated by the combination index (C.I.) analysis. The C.I. was calculated as follows: C.I.=D1/Df1+D2/Df2+D1D2/Df1Df2. Where. Df1 is the dose of Drug-1 required to produce x percent effect alone and D1 is the dose of Drug-1 required to produce the same x percent effect in combination with Drug-2; similarly, Df2 is the dose of Drug-2 required to produce x percent effect alone and D2 is the dose of Drug-2 required to produce the same x percent effect in combination with Drug-1. Theoretically, C.I. is the ratio of the combination dose to the sum of the single-drug doses at an isoeffective level. Consequently, C.I. values <1 indicate synergism, values >1 show antagonism, and values=1 indicate additive effects.

Statistical analysis: The statistical analysis of the samples was undertaken using a Student's t-test, and p-values <0.05 were considered statistically significant. All data reported are means±standard deviations, unless otherwise noted.

### Example 8

Platinum Prodrug Conjugated with Photosensitizer from AIE Characteristics for Drug Activation Monitoring and Combinatorial Photodynamic-Chemotherapy Against Cisplatin Resistant Cancer Cells

A targeted platinum(IV) prodrug conjugated with a mono-functionalized AIE PS for selectively and real-time monitoring of drug activation in-situ as well as the combinatorial photodynamic-chemotherapy against cisplatin resistant cancer cells was developed. The two axial positions of the platinum(IV) prodrug were modified with an AIE PS and a hydrophilic peptide with dual functions to endow the targeting ability and water solubility of the prodrug (FIG. 22). FIG. 22 illustrates (A) Chemical structure of the prodrug TPECB-Pt-D5-cRGD; (B) Schematic illustration of TPECB-Pt-D5-cRGD used for cisplatin activation monitoring and image-guided combinatorial photodynamic therapy and chemotherapy for the ablation of cisplatin resistant cancer cells.

The prodrug is non-emissive in aqueous media and can be uptake by αvβ3 integrin overexpressed cancer cells through receptor mediated endocytosis. Then the prodrug can be activated by intracellular glutathione (GSH) concomitantly with the fluorescence turn-on from the released AIE PS, which can be used for drug activation monitoring and cancer cell imaging. Upon image-guided light irradiation, the AIE PS can generate ROS efficiently for photodynamic therapy. Our prodrug design thus offers good opportunity for prodrug activation monitoring and image-guided chemo-photodynamic combination therapy for cisplatin-resistance cancer cells.

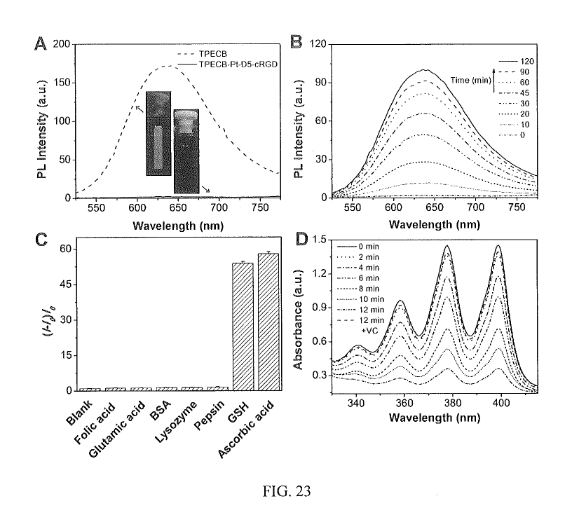

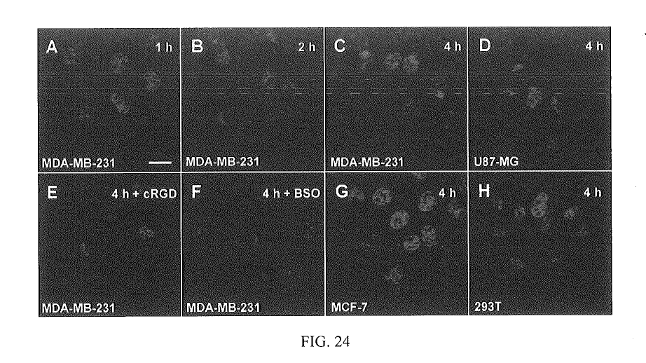

FIG. 23 illustrates (A) Photoluminescence (PL) spectra of TPECB and TPECB-Pt-D5-cRGD (10 μM) in DMSO/PBS (v/v=1/199). Inset shows the photographs taken under a hand-held 365 nm lamp. (B) Fluorescence spectra of TPECB-Pt-D5-cRGD (10 μM) incubated with GSH (100 μM) in DMSO/PBS (v/v 1/199) after different time durations. (C) Fluorescence response of TPECB-Pt-D5-cRGD (10 μM) toward 100 μM of different analysts in DMSO/PBS (v/v=1/199). (D) UV-vis absorption changes of ROS indicator 9,10-anthracenediyl-bis(methylene)dimalonic acid (ABDA) mixed with GSH-pretreated prodrug for different time duration of light irradiation. VC stands for ROS scavenger vitamin C. Data represent mean values±standard deviation, n=3. The selectivity of TPECB-Pt-D5-cRGD towards other biological related analytes was studied by monitoring the fluorescence change. As shown in FIG. 23C, an intense fluorescence increase was only observed when the prodrug was incubated with reducing agent (GSH or ascorbic acid), indicating the high selectivity of the prodrug.

The ROS generation of the AIE residue was studied by measuring the absorption spectra of the mixture of TPECB-Pt-D5-cRGD and 9,10-anthracenediyl-bis(methylene)dimalonic acid (ABDA) in DMSO/PBS (v/v=1/199) upon light irradiation. It should be noted that the absorbance of TPECB-Pt-D5-cRGD is low and will not disturb the absorbance change of ABDA. As depicted in FIG. 23D, the absorption peaks at 358 nm, 378 nm and 400 nm attributed to the anthracene moiety in ABDA decreased gradually upon light irradiation. This is due to the fact that ABDA can efficiently trap ROS by fast reaction with the anthracene moiety. Upon light irradiation, the absorption at 400 nm is significantly decreased from 100% to 24.1% of its original value after 12 min of light irradiation (0.25 W cm-2).

The drug activation of TPECB-Pt-D5-cRGD in cells was studied by incubating the prodrug with MDA-MB-231 and U87-MG cancer cells with overexpressed αᵥβ₃ integrin and MCF-7 cancer cells with low integrin αᵥβ₃ expressed as well as 293T normal cells as the negative control. FIG. 24 illustrates confocal images of prodrug incubated MDA-MB-231 cells (A-C, E, F), U87-MG cells (D), MCF-7 cells (G), 293T cells (H) for different time durations. For E and F, the cells were pretreated with free cRGD or buthionine sulfoximine (BSO), respectively. The red fluorescence is from TPETB (Ex: 405 nm; Em: >560 nm); the blue fluorescence is from cell nucleus dyed with Hoechst (Ex: 405 nm; Em: 430-470 nm). All images share the same scale bar (20 μm).

As shown in FIG. 24, the red fluorescence attribute to the cleaved AIE residues in prodrug incubated MDA-MB-231 cells increases gradually with incubation time, which was also confirmed by flow cytometric studies. However, when the prodrug was incubated with cRGD-pretreated MDA-MB-231 cells, the red fluorescence signal is very weak after 4 h incubation, indicating that the prodrug was uptaken by the cells through receptor-mediated endocytosis. When the MDA-MB-231 cells were pretreated with buthionine sulfoximine (BSO) to inhibit GSH synthesize in the cells, the fluorescence is also significantly decreased. The result reveals that the fluorescence is directly related to the intracellular GSH concentration, which is the major reducing agent for drug activation. The U87-MG cells also showed intense red fluorescence after 4 h incubation. Only weak fluorescent signals in MCF-7 and 293T cells can be detected after 4 h incubation, which should be due to low αᵥβ₃ integrin expressed on the cell surface. The flow cytometric studies also confirmed that the prodrug uptake is more for MDA-MB-231 cells than MCF-7 and 293T cells.

Upon light irradiation, the ROS generation of the AIE residues in the cells was studied using a cell permeable fluorescent ROS indicator 2′,7′-dichlorodihydrofluorescein diacetate (DCF-DA). DCF-DA is non-fluorescent but can be readily oxidized by ROS to the highly fluorescent product dichlorofluorescein (DCF). The fluorescent signal of DCF decreases significantly when VC was added, further confirming the generation of ROS upon light irradiation.

Under light irradiation, the toxicity of TPECB-Pt-D5-cRGD to MDA-MB-231, U87-MG, MCF-7 and 293T cells were studied. After incubation of the prodrug with the cells for 4 h, the prodrug was removed by washing with fresh medium and further exposed with light irradiation and stained with FITC-tagged Annexin V, which is a fluorescent indicator to distinguish apoptotic cells from viable cells. MDA-MB-231 and U87-MG cells show strong green fluorescence from FITC, indicating that the MDA-MB-231 and U87-MG cells are undergoing apoptosis process.

Subsequently, the anti-proliferative properties of TPECB-Pt-D5-cRGD towards MDA-MB-231 cells, U87-MG cells, MCF-7 cells and 293T cells were evaluated by MTT assays. The MDA-MB-231 cells are cisplatin resistant while U87-MG cells are cisplatin-sensitive. This is also evidenced by the half-maximal inhibitory concentration (IC₅₀) of cisplatin to MDA-MB-231 cells is 33.4 μM, which is comparable to that of the cisplatin resistance cancer cells. In contrast, the IC₅₀ value of cisplatin to U87-MG cells is much lower (5.4 μM). It should be noted that the cytotoxicity of cisplatin to both cells was not affected by the light irradiation. The prodrug TPECB-Pt-D5-cRGD showed similar cytotoxicity with cisplatin to MDA-MB-231 cells under dark conditions (37.1 μM), but its cytotoxicity was enhanced remarkably upon light irradiation (IC₅₀=4.2 μM). These results clearly demonstrated hat the anti-proliferative effect of TPECB-Pt-D5-cRGD against cisplatin-resistant MDA-MB-231 cancer cells has been greatly enhanced by the synergistic effect achieved via both chemotherapy and photodynamic therapy. In contrast, the prodrug shows minimum cytotoxicity to MCF-7 and 293T cells in dark or with light irradiation.

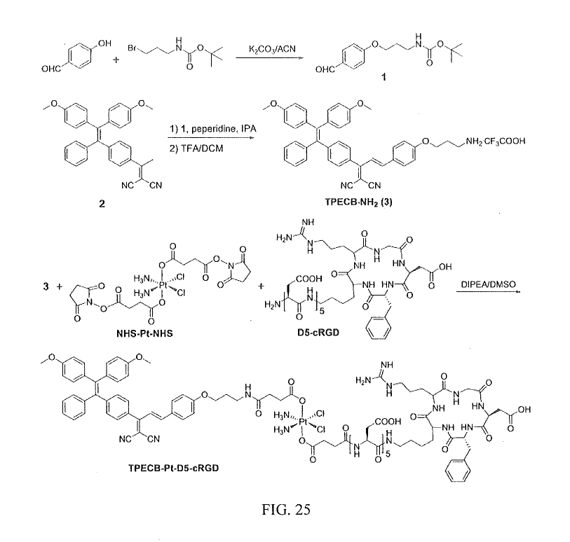

The synthetic route is described in FIG. 25

### Synthesis of Compound 1

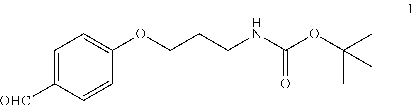 [SMILES: CC(C)(C)OC(=O)NCCCOc1ccc(C=O)cc1]

To the solution of 4-hydroxybenzaldehyde (360 mg, 2.95 mmol) in acetonitrile (10 mL) was added tert-butyl N-(3-bromopropyl)carbamate 980 mg, 4.11 mmol) and K2CO3 (480 mg, 3.48 mmol). The resulting mixture was stirred at reflux overnight. After the mixture was cooled down to room temperature, the mixture was filtered and the filtrate was concentrated and purified with chromatography (hexane:ethyl acetate v/v=3:1) to give the desired product (colorless oil, 390 mg, 47.4%). ¹H NMR (300 MHz, CDCl3) δ 9.85 (s, 1H), 7.81 (dd, J1=1.6 Hz, J2=5.6 Hz, 2H), 6.98 (dd, J1=1.6 Hz, J2=5.6 Hz, 2H), 4.78 (brs, 1H), 4.08 (t, J=4.8 Hz, 2H), 3.32 (m, 2H), 2.02 (m, 2H), 1.41 (s, 9H).

### Synthesis of TPECB-NH₂.

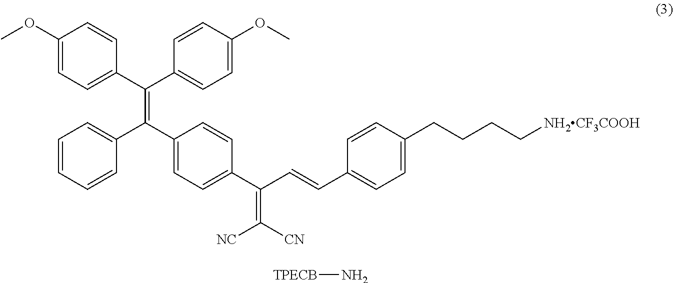

To the solution of compound 2 (40 mg, 0.083 mmol) in isopropanol (5 mL) was added compound 1 (30 mg, 0.11 mmol) and piperidine (0.68 mg, 0.008 mmol). The resulting solution was refluxed for 24 hours. Then the solvent was removed under reduced pressure. The desired residue was purified with chromatography (hexane: ethyl acetate v/v=5:1) to give a red oil. This oil was further treated with the mixture of dichloromethane (5 mL) and trifluoroacetic acid (1 mL) for 8 hours. Then the solvent was removed under reduced pressure. The residue was purified with reverse HPLC using acetonitrile and water as the mobile phase to give the desired product (yellow solid, 12 mg, 23.0%). ¹H NMR (400 MHz, DMSO-d6) δ 7.79 (brs, 2H), 7.63 (d, J=8.8 Hz, 2H), 7.40 (d, J=15.2 Hz, 1H), 7.27 (d, J=8.4 Hz, 2H), 7.13-7.20 (m, 2H), 7.15 (m, 3H), 7.02-7.06 (m, 4H), 6.87-6.92 (m, 4H), 6.67-6.73 (m, 5H), 4.16 (d, J=6.0 Hz, 2H), 3.68 (s, 6H), 2.95-3.00 (m, 2H), 2.00-2.04 (m, 2H). MS (ESI) calcd for [M+H]+: 644.2913, found: 644.2926.

### Synthesis and Purification of the Prodrug TPECB-Pt-D5-cRGD

In a typical reaction, TPECB-NH2 (5.0 mg, 7.8 μmol) and amine-functionalized D5-cRGD (9.2 mg, 7.8 μmol) were dissolved in anhydrous DMSO (0.5 mL) with DIPEA (1.0 μL) and the mixture was stirred at room temperature for 10 min. Then NHS-Pt-NHS (5.6 mg, 7.8 μmol) in anhydrous DMSO (0.5 mL) was added quickly to the above solution. The reaction was continued with stirring at room temperature for another 24 h. The final product was purified by prep-HPLC (solvent A: water with 0.1% TFA, solvent B: CH3CN with 0.1% TFA) and lyophilized under vacuum to yield the prodrug as yellow powders in 38% yield (6.6 mg).

General procedure for drug activation monitoring. DMSO stock solution of TPECB-Pt-D5-cRGD (1 mM) were diluted into a mixture solvent of DMSO and PBS (v/v=1/199). Then the prodrug was incubated with GSH at room temperature and the fluorescence change was studied. The solution was excited at 405 nm, and the emission was collected from 525 to 775 nm.

### Example 9

### Light-Harvesting Conjugated Polyelectrolytes (CPEs)

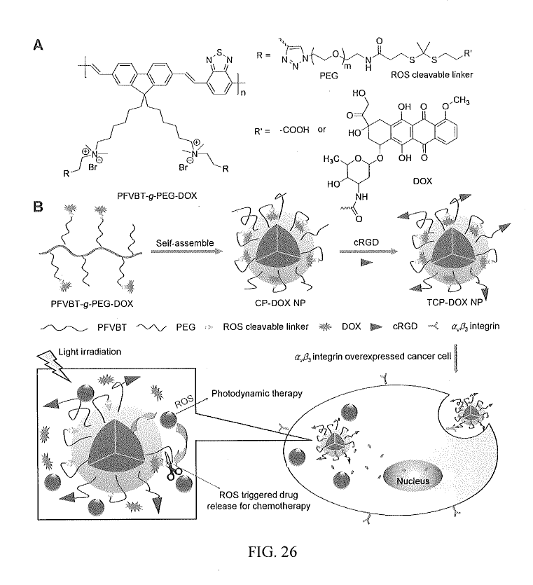

A CPE-doxorubicin (DOX) conjugate polyprodrug for targeted cell imaging guided on-demand photodynamic therapy and chemotherapy upon one light irradiation was developed. The anticancer drug DOX was covalently conjugated to a PEGylated polymeric photosensitizer CPE through a ROS cleavable linker. FIG. 26 is an illustration of (A) Chemical structure of the PEGylated polyprodrug PFVBT-g-PEG-DOX and (B) schematic illustration of the light regulated ROS activated on-demand drug release and the combined chemo-photodynamic therapy. The obtained polyprodrug could self-assemble into nanoparticles (NPs) in aqueous media and the surface was further functionalized with cRGD that targets αvβ3 integrin overexpressed cancer cells. Under white light irradiation, these NPs can generate ROS efficiently for photodynamic therapy. Meanwhile, the generated ROS around the NPs can quickly cleave the linker that covalently linked to the chemotherapeutic drug for specific on-demand drug release. As compared to the existing systems, our “all-in-one” polyprodrug based on a single CPE contains all the functionalities for imaging, therapy and on-demand drug release. It possesses the following advantages: (1) smart design of a polymeric photosensitizer as drug carrier, (2) targeted cancer cell imaging for imaging guided therapy, (3) efficient ROS generation under light irradiation for photodynamic therapy, (4) on-demand drug release for chemotherapy, and (5) single light controlled chemo- and photodynamic combination therapy for efficient cancer treatment.

The synthesis of PFVBT-g-PEG-DOX is as follows. First, the ROS cleavable thioketal (TK) linker was prepared and one of its carboxyl groups was reacted with the amine group of a bifunctional poly(ethylene glycol) (N3-PEG-NH2) to yield N3-PEG-TK. The carboxyl group of N3-PEG-TK was further reacted with the amino group of DOX. After reaction, the mixture was dialyzed and freeze dried to yield the product denoted as N3-PEG-TK-DOX. An equal molar of N3-PEG-TK and DOX was used for conjugation and about 70% of the carboxyl groups were reacted based on NMR spectra. The unreacted carboxyl groups allowed for further attachment of target moiety after polymer self-assembly. On the other hand, poly[9,9-bis(N-(but-3′-ynyl)-N,N-dimethylamino)hexyl))fluorenyl divinylene-alt-4,7-(2′,1′,3′-benzothiadiazole) dibromide] (PFVBT) with alkyne side groups was synthesized according to our previous reports. This polymer allows for subsequent click reaction with azide functionalized N3-PEG-TK-DOX to yield the brush copolymer PFVBT-g-PEG-DOX. The DOX content in the conjugate was calculated to be 12.3 wt % based on the integrated areas between the peak at 3.62 ppm (assigned to the methylene protons of PEG) and the peak at 0.56 ppm (assigned to the methylene protons secondly close to the 9-position of fluorene) in the NMR spectrum. Brush polymer without conjugation of DOX was also prepared and denoted as PFVBT-g-PEG.

High performance liquid chromatography (HPLC) was used to monitor the drug release from N3-PEG-TK-DOX in the presence of ROS, which was produced by reacting H₂O₂ with Fe²⁺. N3-PEG-TK-DOX exhibits a monodispersed peak at an elution time of 3.5 min. Since the elution of HPLC has 0.1% trifluoroacetic acid, we also incubated N3-PEG-TK-DOX in water at pH 1.0 for 6 h, which showed no degradation of the compound, demonstrating good stability of the thioketal linker under acidic conditions. Treatment of N3-PEG-TK-DOX with ROS completely degraded the thioketal linker, resulting in a single peak with an elution time of 4.9 min, which shows a mass-to-charge ratio (m/z) of 632.266 determined from ESI-Mass, corresponding to the sulfhydryl modified doxorubicin. Although a short thiol ligand (3-mercapto-propanone) is attached to DOX after the drug release, previous reports demonstrated that the DOX derivative did not reduce the potency of the drug.

The PFVBT-g-PEG-DOX can self-assemble into micellar NPs through a dialysis method (denoted as CP-DOX NPs). As carboxyl groups are located at the terminal of the hydrophilic PEG side chain, upon NP formulation, the carboxyl groups should present on the NP surface, making them available for surface chemistry. NPs can be further functionalized with a cyclic arginine-glycine-aspartic acid (cRGD) tripeptide for targeting integrin αvβ3 overexpressed cancer cells to achieve cancer-targeted drug delivery. The target CP-DOX NPs are denoted as TCP-DOX NPs. NPs self-assembled from PFVBT-g-PEG denoted as TCP NPs. The TCP-DOX NPs have an absorption maximum at 502 nm and an emission maximum at 598 nm with a Stokes shift of ˜96 nm. The hydrodynamic diameter of TCP-DOX NPs was investigated by laser light scattering (LLS), which shows a volume average hydrodynamic diameter of 120±11 nm.

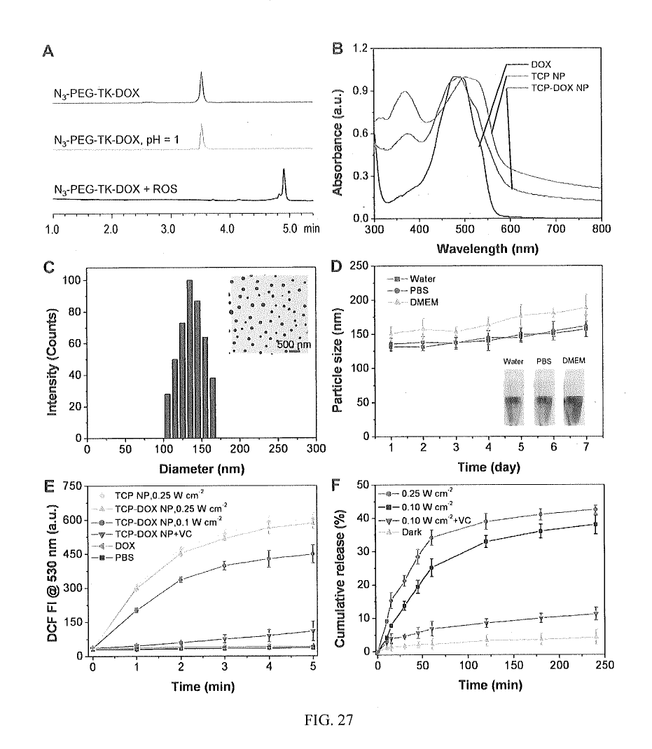

FIG. 27 is (A) Analyses of the stability and degradation of N3-PEG-TK-DOX in the presence of ROS detected at absorbance of 254 nm by HPLC. (B) Normalized UV-vis absorption spectra of DOX, TCP NPs and TCP-DOX NPs. (C) Size distribution and TEM image (inset) of TCP-DOX NPs. (D) Average hydrodynamic diameter changes of TCP-DOX NPs when incubated in water, PBS buffer or DMEM at 37° C. for 7 days (the inset digital photograph shows TCP-DOX NPs dispersed in water, PBS buffer or DMEM, indicating good dispersity). (E) Dichlorofluorescein (DCF) fluorescence intensity at 530 nm in PBS, DOX, TCPDOX NPs and TCP NPs after light irradiation for different time. VC stands for ROS scavenger vitamin C. (F) Cumulative release profiles of DOX from TCPDOX NPs without and with the light irradiation. Standard deviations are shown as error bars from three parallel experiments.

The ROS generation was determined by the fluorescence signal of a ROS-sensitive probe, dichlorofluorescein diacetate (DCF-DA). DCF-DA is non-fluorescent, but it can be rapidly oxidized to a fluorescent molecule (dichlorofluorescein, DCF) by ROS. Since PFVBT has a broad absorption spectrum, white light is able to induce the production of ROS. The ROS production is more efficient with the increased power density. Upon irradiation of TCP-DOX NPs for 5 min at a power density of 0.1 W cm-2, a 11.5-fold enhancement in fluorescence intensity of DCF is detected at 530 nm, while the control groups without the NPs remains at the original level. When vitamin C (VC, a well-known ROS scavenger) was added, the fluorescence from the DCF was remarkably inhibited, further confirming the ROS generation after light irradiation.

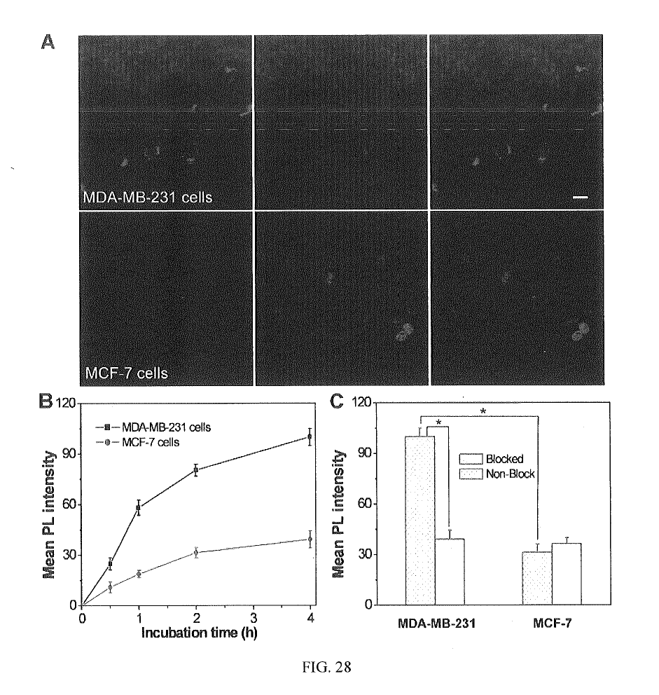

To demonstrate the feasibility of achieving cancer targeted delivery of DOX, TCP-DOX NPs were incubated with MDA-MB-231 and MCF-7 cancer cells expressing different levels of αvβ3 integrin receptor and the fluorescence of PFVBT-g-PEGDOX were monitored at different incubation time points. MDAMB-231 cells with overexpressed integrin αvβ3 on cellular membrane were chosen as integrin-positive cancer cells, while MCF-7 cells with low αvβ3 integrin expression were used as the negative control. The confocal imaging results are shown in FIG. 28. FIG. 28 is evaluation of the targeting effect of TCP-DOX NPs to different cancer cells: (A) Confocal microscopy images of MDA-MB-231 and MCF-7 cells after incubation with the NPs for 4 h. The blue fluorescence is from the nuclei of cells stained by Hoechst 33342, the red fluorescence is from PFVBT-g-PEG-DOX. All images share the same scale bar (20 μm); (B) Integrated fluorescence intensity of PFVBT-g-PEG-DOX determined in MDA-MB-231 and MCF-7 cells at different incubation time; (C) fluorescence intensity of PFVBT-g-PEG-DOX determined in MDA-MB-231 and MCF-7 cells with and without cRGD (50 μM) pretreatment. The error is the standard deviation from the mean (n=3, * is P<0.05).

After 4 h incubation, both red fluorescence from PFVBT-g-PEG-DOX in cytoplasm and blue emission from Hoechst in cell nucleus were observed in MDA-MB-231 cells, which are much brighter than those in MCF-7 cells. Semi-quantitative fluorescence intensity analysis of red fluorescence in these cells confirms that the uptake of cRGD modified NPs in MDA-MB-231 cells is 2.9 times higher than that in MCF-7 cells (FIG. 28B). We also noticed that the fluorescence intensities in both cells gradually enhanced with the increase of incubation time, and at each time point, a higher fluorescence is observed in MDA-MB-231 cells. FIG. 28C shows that the fluorescent signal is dramatically reduced in MDA-MB-231 cells when integrin was initially blocked by excess cRGD. Semi-quantitative fluorescence analysis in MDA-MB-231 cells demonstrates that there is significant difference (p<0.05) in the cellular uptake of TCP-DOX NPs when integrin was blocked, indicating the αvβ3 integrin receptor mediated cell uptake. Under light irradiation, the ROS production by TCP-DOX NPs inside the cancer cells was studied using a cell permeable fluorescent dye DCF-DA. Strong green fluorescence of DCF is observed when the cells are loaded with TCP-DOX NPs and after light irradiation. When ROS scavenger VC (50 μM) is added, the fluorescent signal of DCF decreases significantly, which further confirms the generation of ROS inside the cells during light irradiation.

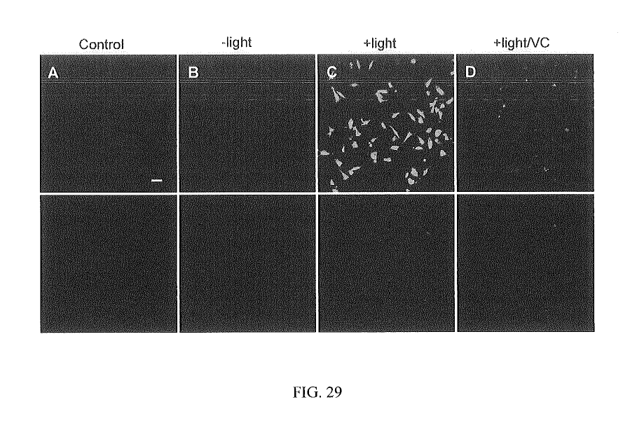

FIG. 29 is detection of intracellular reactive oxygen species (ROS) production using DCF-DA staining in MDA-MB-231 cells incubated with (A) DCF-DA; (B) TCP-DOX NPs; (C) TCP-DOX NPs and DCF-DA; (D) TCP-DOX NPs and DCFDA in the presence of ROS scavenger (VC, 50 μM). Green: ROS indicator DCF; Red: PFVBT-g-PEG-DOX fluorescence. All images share the same scale bar (50 μm).

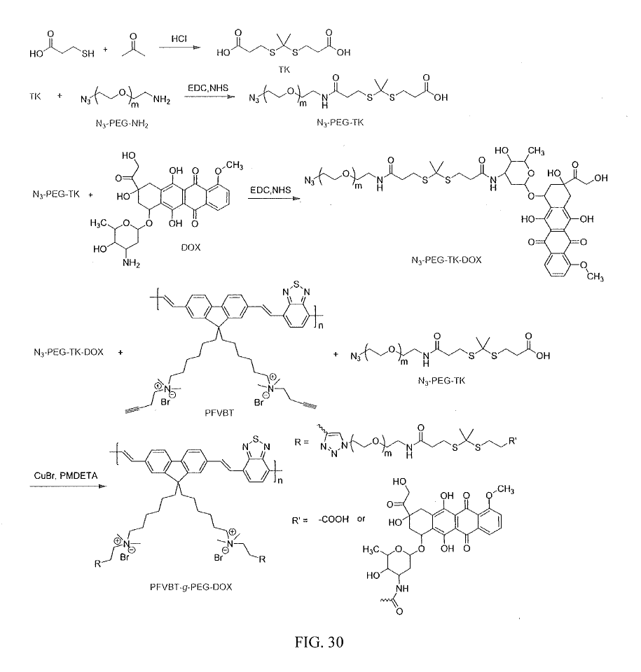

FIG. 30 is the synthetic scheme of PFVBT-g-PEG-DOX.

### Synthesis of ROS-Cleavable Thioketal Linker (TK)

In a typical reaction, a mixture of anhydrous 3-mercaptopropionic acid (5.2 g, 49.1 mmol) and anhydrous acetone (5.8 g, 98.2 mmol) were saturated with dry hydrogen chloride and stirred at room temperature for 6 h. After the reaction, the flask was stoppered and chilled in an ice-salt mixture until crystallization was complete. The crystals were filtered, washed with hexane and cold water, the product was obtained after drying in a vacuum desiccator (80%). ¹H NMR (400 MHz, CD3OD, δ): 2.85 (t, 4H), 2.58 (t, 4H), 1.58 (s, 6H). ESI-MS (m/z): [M+H]+ calcd, 252.049; found, 252.140.

### Synthesis of N3-PEG-TK Conjugate.

A mixture of N3-PEG-NH2 (205.9 mg, 0.1 mmol) and TK (252.1 mg, 1.0 mmol) in anhydrous DMF (2 mL) was stirred at room temperature for 10 min. Then EDC (57.3 mg, 0.3 mmol) and NHS (34.5 mg, 0.3 mmol) dissolved in anhydrous DMF (1 mL) was added to the above solution under nitrogen atmosphere. The reaction was performed under nitrogen atmosphere for 24 h at room temperature. After that, the reaction mixture was extensively dialyzed (SpectraPor 6, molecular weight cutoff of 1,000) against deionized water to remove EDC and NHS. The polymer was obtained as white powders after freeze-drying under vacuum. Then the crude product was redissolved in DMF (1 mL) and dropped into 100 mL of cold diethyl ether under stirring to precipitate the N3-PEG-TK conjugate. This procedure was repeated once more and the final product was obtained after dried in vacuum (75%). ¹H NMR (400 MHz, CDCl3, δ): 3.58-3.72 (m, 160H), 2.87 (t, 4H), 2.63 (t, 2H), 2.54 (t, 2H), 1.58 (s, 6H).

### Preparation of N3-PEG-TK-DOX Conjugate.

The carboxyl group of N3-PEG-TK was conjugated with the amine group of DOX under the catalysis of EDC and NHS according to a similar procedure. Briefly, a mixture of N3-PEG-TK (112.1 mg, 48.7 μmol), doxorubicin (28.2 mg, 48.7 μmol) and triethylamine (14.1 μL, 97.4 μmol) in anhydrous DMF (1 mL) was stirred at room temperature for 10 min to obtain a clear solution. Then EDC (18.6 mg, 97.4 μmol) and NHS (11.2 mg, 97.4 μmol) dissolved in anhydrous DMF (1 mL) was added to the above solution under nitrogen atmosphere. The reaction was performed under nitrogen atmosphere at room temperature for 24 h. After that, unreacted DOX was removed by dialyzing the mixture against DMSO (SpectraPor 6, molecular weight cutoff=1,000) with further ultrafiltration against Milli-Q water and freeze-dried under vacuum to obtain N3-PEG-TK-DOX conjugate.

### Synthesis of PFVBT-g-PEG-DOX

PFVBT (6 mg, 10 μmoL alkyne) and N3-PEG-TK-DOX (56.6 mg, 20 μmoL) were dissolved in DMF (5 mL). The mixture was degassed, and then N, N, N′, N″, N′″-pentametyldiethylenetriamine (PMDETA) (3.5 mg, 20 μmoL) and CuBr (2.9 mg, 20 μmoL) were added. After reaction at 65° C. under nitrogen for 24 h, the reaction mixture was cooled to room temperature and filtered through a 0.45 μm syringe driven filter. The filtrate was precipitated into a mixture of methanol and diethyl ether (v/v=1/5) three times to give red powders. The crude product was redissolved in DMF and further purified by dialysis against distilled water using a Spectra/Por dialysis tubing (molecular weight cutoff of 12,000 Da, Spectrum Laboratories, Rancho Dominguez, Calif., United States) for 48 h with changes of water. After freeze-drying, PFVBT-g-PEG-DOX (30.1 mg, 48%) was obtained as red powders. ¹H NMR (400 MHz, DMSO-d6, δ): 8.35-7.65 (m, 18H), 5.20 (s, 0.9H), 5.02 (m, 0.9H), 4.58 (d, 0.9H), 4.15-4.09 (m, 0.9H), 3.97 (s, 2H), 3.78-3.46 (m, 120H), 2.96 (m, 10H), 2.84-2.56 (m, 5H), 2.24-2.12 (m, 2H), 1.58 (s, 3.5H), 1.29-0.95 (m, 12H). 0.92-0.78 (m, 6H), 0.56 (br, 4H).

### Preparation of the Nanoparticles.

The nanoparticles of the brush copolymers were prepared by a dialysis method. In a typical process, 2 mg of the brush copolymer was dissolved in 2 mL of DMSO. Under moderate stirring, the predetermined volume (3 mL) of ultrapurified water (Millipore, 18.2 MΩ) was added slowly. The mixture was left stirring for an additional 3 h. The solvents were then removed by dialysis (molecular weight cutoff of 3,500 Da, Spectrum Laboratories, Rancho Dominguez, Calif., USA) against Milli-Q water to obtain the nanoparticles. The final volume was adjusted to 2 mL by ultrafiltration (20,000 MWCO, Amicon, Millipore Corporation, Bedford, USA) for further experiments.

Conjugation of cRGD to the Nanoparticles.

Amine functionalized cRGD was conjugated to the surface of the CP-DOX NPs using an EDC/sulfo-NHS technique. The nanoparticles were suspended in deionized water (0.2 mg mL-1) and incubated with excess EDC (10 mM) and Sulfo-NHS (5 mM) at room temperature for 30 min. The resulted sulfo-NHS activated, nanoparticles were washed with Milli-Q water (3 mL×3 times) by ultrafiltration (20,000 MWCO, Amicon, Millipore Corporation, Bedford, USA) to remove the residual EDC and sulfo-NHS. The activated nanoparticles were allowed to react with amine functionalized cRGD (0.1 mg mL-1 in Milli-Q water) for 4 h under magnetic stirring. The cRGD functionalized nanoparticles were washed with Milli-Q water (3 mL×3 times) by ultrafiltration (20,000 MWCO, Amicon, Millipore Corporation, Bedford, USA), resuspended in Milli-Q water and stored at 4° C. for further use.

### Example 10

### Self-Assembled Theranostic Platform Based on PEGylated CPE

In another example embodiment, the present invention is a multifunctional nanoparticle based on PEGylated CPE, which serves as a chemotherapeutic drug carrier for targeted cancer cell imaging and chemotherapy and photodynamic therapy. The PEGylated CPE can easily self-assemble into NPs in aqueous media which can encapsulate commonly used hydrophobic chemotherapeutic drugs, such as paclitaxel (PTX) through hydrophobic-hydrophobic interaction. In addition, the polymer matrix itself can also serve as a photosensitizing unit for imaging and photodynamic therapy. To improve the specificity of the system, recognition element cyclic arginineglycine-aspartic acid (cRGD) tripeptide which is target to integrin αvβ3 overexpressed cancer cells was incorporated onto the self-assembled NPs for targeted cancer therapy. By combining these capabilities, the drug-loaded PEGylated CPE platform has the following distinct advantages: 1) easy to fabricate; 2) imaging guided therapy; 3) dual therapy (photodynamic therapy and chemotherapy) and 4) target ability.

The CPE of poly[9,9-bis(N-(but-3′-ynyl)-N,N-dimethylamino)hexyl)) fluorenyl divinylene-alt-4,7-(2′,1′,3′,-benzothiadiazole) dibromide] (PFVBT) with alkyne side groups was synthesized according to methods known to skill in the art. Subsequent click reaction between the polymer and α-azide-ω-caboxyl-poly(ethylene glycol) (N3-PEG-COOH) using copper(I) bromide (CuBr) and N, N, N′, N″, N′″-pentametyldiethylenetriamine (PMDETA) as the catalyst yielded the PEGylated brush copolymer of PFVBT-g-PEG, which corresponds to the structure below:

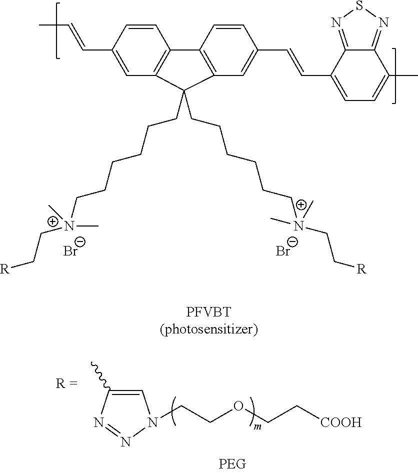

The PFVBT-g-PEG with hydrophobic backbone and hydrophilic PEG side chain can self-assemble into NPs in aqueous solution. The NPs encapsulated with hydrophobic anticancer drug paclitaxel (PTX) were prepared by a dialysis method to yield CP/PTX NPs. As the carboxyl group is located at the terminal end of the hydrophilic PEG block; upon NP formulation, the carboxyl groups should be exposed for subsequent surface chemistry. The NPs were also further functionalized with a cancer targeting cRGD tripeptide (denoted as TCP/PTX NPs) for targeting integrin αvβ3 overexpressed cancer cells to achieve cancertargeted drug delivery. The targeted NPs without loading of PTX were denoted as TCP NPs.

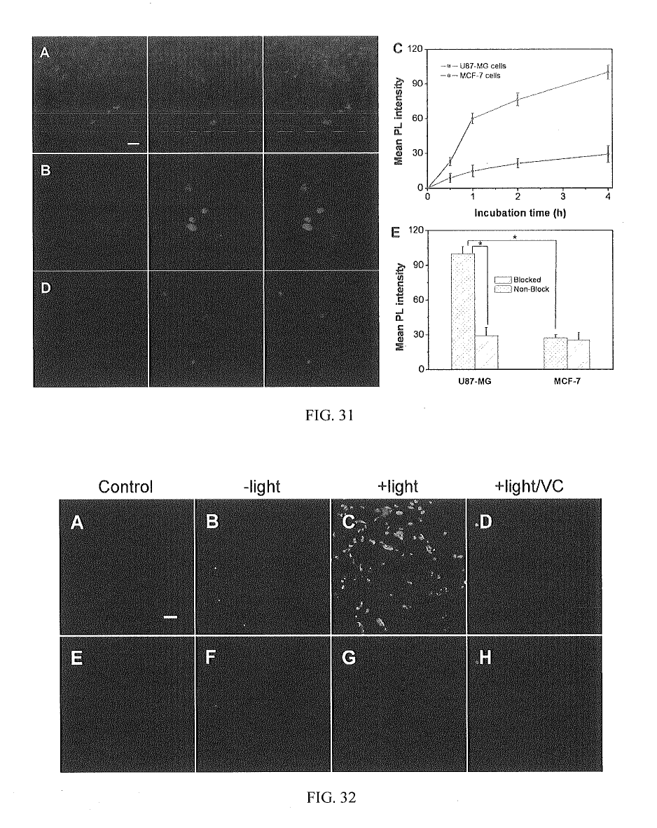

FIG. 31 illustrates the targeting effect of TCP/PTX NPs to different cancer cells: (A-B) confocal microscopy images of NPs uptake in U87-MG cells (A) with receptor overexpression and receptor negative MCF-7 cells (B), the images can be classified to blue fluorescence from the nuclei of cells dyed by Hoechst 33342, red fluorescence from TCP/PTX NPs, and the merged images of above. All images share the same scale bar (20 μm); (C) dynamic fluorescence intensity of TCP/PTX NPs determined in U87-MG and MCF-7 cells at different incubation time points; (D) confocal microscopy images of TCP/PTX NPs uptake in cRGD (50 μM) pretreated U87-MG cells and (E) mean fluorescence intensity of TCP/PTX NPs determined in U87-MG and MCF-7 cells with receptor blocking or nonblocking after 4 h incubation. The error is the standard deviation from the mean (n=3, * is P<0.05).

FIG. 32 illustrates detection of intracellular reactive oxygen production (ROS) by DCF-DA staining in U87-MG cells incubated with (A) DCF-DA with light excitation; (B) TCP/PTX NPs with light excitation; (C) TCP/PTX NPs and DCF-DA with light excitation; (D) TCP/PTX NPs and DCF-DA in the presence of ROS scavenger (vitamin C, 50 μM) with light excitation. E-H indicate the corresponding CP fluorescence. All images share the same scale bar (50 μM).

To evaluate the ROS production by TCP/PTX NPs after cancer cell uptake, we detected the ROS generation under light irradiation using a cell permeable fluorescent dye dichlorofluorescein diacetate (DCF-DA). As shown in FIG. 32, there is negligible fluorescence background when the cells are only loaded with DCF-DA or TCP/PTX NPs with the light irradiation. However, when the cells are loaded with both DCF-DA and TCP/PTX NPs, after the light irradiation, strong green fluorescence of DCF was observed inside the cells, demonstrating the efficient ROS generation from the TCP/PTX NPs. However, when ROS scavenger vitamin C (50 μM) is added, the fluorescence signal of DCF decreases significantly (FIG. 32D), further confirming the generation of ROS inside the cells during light irradiation process.

The biocompatibility of a drug delivery system is crucial for biomedical applications. We first tested the in vitro toxicity of the PFVBT-g-PEG nanoparticles without PTX loading (TCP NPs) in the dark. The standard methyl thiazolyl tetrazolium (MTT) assay was firstly carried out to determine the relative viabilities of U87-MG and MCF-7 cells after they were incubated with TCP NPs at various concentrations for 24 h and 48 h. No significant cytotoxicity of TCP NPs is observed for both cells even at high concentrations of up to 0.2 mg mL-1. To further look for any potential cell damage caused by the TCP NPs, the release of lactate dehydrogenase (LDH), an indicator of cell membrane damage, was also examined. Cells lysed by 1% Triton X-100 were used as positive controls.

### Example 11

Cellular and Mitochondria Dual Target Organic Dots with AIE Characteristics for Image-Guided Photodynamic Therapy

In another example embodiment, the present invention is targeted delivery of therapeutic agents towards organelles of targeted cancer cells. In another embodiment, the organelle is a mitochondria. Herein, the cellular and mitochondria dual-targeted organic dots for image-guided PDT based on a fluorogen with aggregation-induced emission characteristics (AIEgen) is reported. The synthesized AIEgen possesses enhanced red fluorescence and improved ROS production in aggregated state. The fabricated AIE dots are functionalized with folic acid and triphenylphosphine (TPP) at surface, which are able to selectively internalize into folate-receptor (FR) positive cancer cells, and subsequently accumulate at mitochondria. The direct ROS generation at mitochondria is found to depolarize mitochondrial membrane, affect cell migration, and lead to cell apoptosis and death with enhanced PDT effects as compared to ROS generated randomly in cytoplasm. This report demonstrates a simple and general nanocarrier approach for cellular and mitochondria dual-targeted PDT, which opens new opportunities for dual targeted delivery and therapy.

The new AIEgen, DPBA-TPE, shows characteristic AIE features. Under light illumination, the molecules emit strong red fluorescence and could efficiently generate ROS in aggregates. The corresponding AIE dots were then fabricated by a modified nano-precipitation method using lipid-PEG as encapsulation matrix. Bearing folic acid and TPP targeting ligands at the surface, the yielded FA-AIE-TPP dots are able to selectively internalize into folate-receptor (FR) positive cancer cells over other cells and subsequently accumulate in mitochondria. The dual targeted FA-AIE-TPP dots showed enhanced PDT effects as compared to sole cellular targeted or mitochondria targeted AIE dots. The NP formulation thus represents a more simple and general strategy for targeted cellular and subcellular delivery.

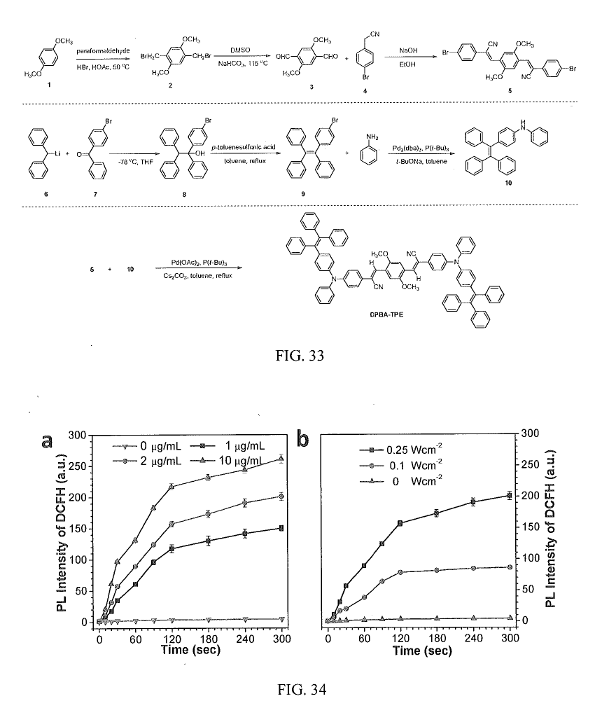

FIG. 33 illustrates the synthetic pathway to create DPBA-TPE.

To demonstrate the potential of AIE dots for cellular and mitochondria dual targeted image-guided PDT, we synthesized a new AIEgen, DPBA-TPE (FIG. 33). 3,3′-(2,5-Dimethoxy-1,4-phenylene)bis(2-(4-bromophenyl)acrylonitrile) (5) was prepared by Knoevenagel reaction from 2,5-dimethoxybenzene-1,4-dicarboxaldehyde (3) and bromophenylacetonitrile (4) under basic conditions. The final product was obtained with satisfactory yields by intermediate (5) and aryl amine (10) in the presence of palladium catalyst under basic conditions.

To fabricate the dual targeting AIE dots, a modified nano-precipitation method was used. Biocompatible block copolymers of lipid-PEG with different terminal groups, (1,2-distearoyl-sn-glycero-3-phosphoethanolamine-N-[amino(polyethylene glycol)-2000]) (DPSE-PEG-NH₂) and (1,2-distearoyl-sn-glycero-3-phosphoethanolamine-N-[folate(polyethylene glycol)-2000]) (DSPE-PEG-FA) were chosen as the encapsulation matrix due to their high loading efficiency, excellent colloidal stability of the formed dots as well as the ability to introduce the surface functional groups. To form the AIE dots, THF solution containing molecularly dissolved DPBA-TPE, DPSE-PEG-NH₂ and DSPE-PEG-FA was diluted into MilliQ water, immediately followed by ultrasound sonication using a microtip sonicator at a power output of 12 W for 120 s. During the mixing and sonication, the hydrophobic DSPE segments will interact and intertwine with the hydrophobic DPBA-TPE to form the core, while the hydrophilic PEG segments will extend outside towards water phase to form the protective shell. The presence of PEG shells not only stabilizes the AIE dots, but also provides the surface amino groups for further conjugation. To bring the AIE dots to mitochondria, cationic TPP, which is able to accumulate in mitochondria in response to high mitochondrial membrane potential (MMP), was then reacted with AIE dot suspension to yield FA-AIE-TPP dots. After the reaction, dialysis of the FA-AIE-TPP dots suspension against water using 6 to 8 kDa membrane is applied to remove excess TPP. Similar procedures were applied to fabricate folic acid mono-functionalized AIE dots (AIE-FA) and TPP mono-functionalized AIE dots (AIE-TPP).

FIG. 34 illustrates ROS generation of FA-AIE-TPP dots in aqueous solution at a) varied dot concentrations, and b) varied light powers upon irradiation for 300 s.

The PDT effect of the AIE dots is further studied by measuring the ROS generation efficiency under light irradiation using DCFH as an indicator. As shown in FIG. 34, the FA-AIE-TPP dot suspension is able to generate ROS very quickly and efficiently under white light irradiation, which is evidenced by the rapid increase of DCFH fluorescence intensity at 530 nm. Moreover, increasing the exposure time, AIE dot concentration, or light power will also increase the ROS generation (FIG. 34), indicating that ROS production of AIE dots is time-, concentration- and power-dependent. Such an efficient ROS generation capability makes the AIE dots a good candidate for image-guided PDT.

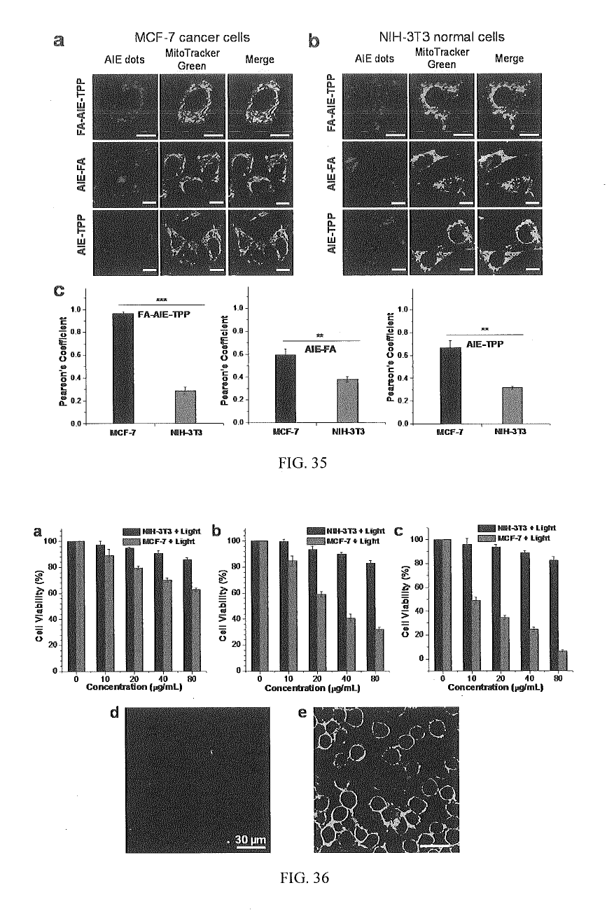

FIG. 35 illustrates CLSM images of a) MCF-7 cancer cells and b) NIH-3T3 normal cells after incubation with AIE dots and MitoTracker Green. AIE dots: Eₓ: 543 nm, Eₘ: >650 nm; MitoTracker Green: Eₓ=488, Eₘ=505-525 nm. c) Pearson's Coefficients between AIE dots and MitoTracker Green inside MCF-7 and NIH-3T3 cells. The scale bar size is 10 μm for all images.

The cellular targeting and mitochondria targeting capabilities of the three AIE dots were investigated by fluorescence imaging. FR-positive MCF-7 breast cancer cells were chosen as the target, with FR-negative NIH-3T3 fibroblast cells as the control. After incubating both cells for 4 h with the three AIE dots at 20 μg/mL based on DPBA-TPE mass concentration, the images were acquired by confocal laser scanning microscope (CLSM). FIG. 35 shows the intracellular localization of these AIE dots in either MCF-7 or NIH-3T3 cells. For FA-AIE-TPP or AIE-FA dots incubated cells, much stronger red fluorescence can be observed for MCF-7 cells than NIH-3T3 cells, revealing the targeting capability of folate decorated AIE dots towards FR-positive cells. However, AIE-TPP dots show very weak fluorescence inside both cell lines.

FIG. 36 illustrates viabilities of MCF-7 cancer cells and NIH-3T3 normal cells after incubation with a) AIE-TPP, b) AIE-FA, c) FA-AIE-TPP dots at varied concentrations, followed by white light irradiation. d) and e) Annexin V labeled MCF-7 cells after incubation with FA-AIE-TPP dots without (d) or with (e) light irradiations. d) and e) share the same scale bar.

The PDT effects of the three AIE dots on viabilities of NIH-3T3 and MCF-7 cells were then investigated by MTT assays. Upon incubation with the three. AIE dots in dark for 24 h, both NIH-3T3 and MCF-7 cells exhibit high cell viabilities of over 90% even at a high DPBA-TPE concentration of 80 μg/mL, indicating the low cytotoxicity of AIE dots without light irradiation. In the parallel experiments, incubating both cell lines with AIE dots for 4 h and followed by light irradiation (100 mWcm⁻²) for 10 min leads to large differences in cell viabilities (FIGS. 36a-c). All three AIE dots exhibit very low photo-toxicity towards NIH-3T3 cells, which should be due to the poor cellular uptake. As for MCF-7 cells, FA-AIE-TPP dots show the most efficient killing efficiency under light irradiation with a cell viability of less than 10% at the DPBA-TPE concentration of 80 μg/mL. While under the same condition, AIE-TPP and AIE-FA dots treated MCF-7 cells show cell viabilities of ˜60% and ˜32%, respectively. The half maximal inhibitory concentration (IC₅₀) was further apply to quantify the anticancer efficiency of the three dots under light irradiation. The IC₅₀ values are >80, ˜32, and ˜10 μg/mL for AIE-TPP, AIE-FA, and FA-AIE-TPP dots, respectively. As almost same amounts of AIE-FA and FA-AIE-TPP dots are internalized into MCF-7 cells as revealed by CLSM and flow cytometry (FIG. 35), the lower IC₅₀ of FA-AIE-TPP dots clearly indicates that localizing PS loaded nanocarriers in mitochondria helps enhance anticancer effects of PDT. The comparison between AIE-TPP and FA-AIE-TPP dots also reveals that the increased cellular uptake also helps increase the amount of NPs accumulated at mitochondria with enhanced PDT. Moreover, the killing efficiency of FA-AIE-TPP dots towards MCF-7 cells also increases with the exposure time and light power. PDT triggered cell death normally destroys the mitochondria membrane and triggers the release of cytochrome, leading to apoptosis process. We used fluorescein isothiocyanate (FITC)-tagged Annexin V to differentiate apoptotic cells from viable ones. As shown in FIGS. 36 d and e, incubation MCF-7 cells with FA-AIE-TPP dots in dark, almost no green fluorescence from Annexin V is observed, while upon light irradiation, bright green fluorescence originated from Annexin V can be observed from cell membrane, indicating that MCF-7 cells undergo apoptosis process in the presence of FA-AIE-TPP dots and light irradiation.

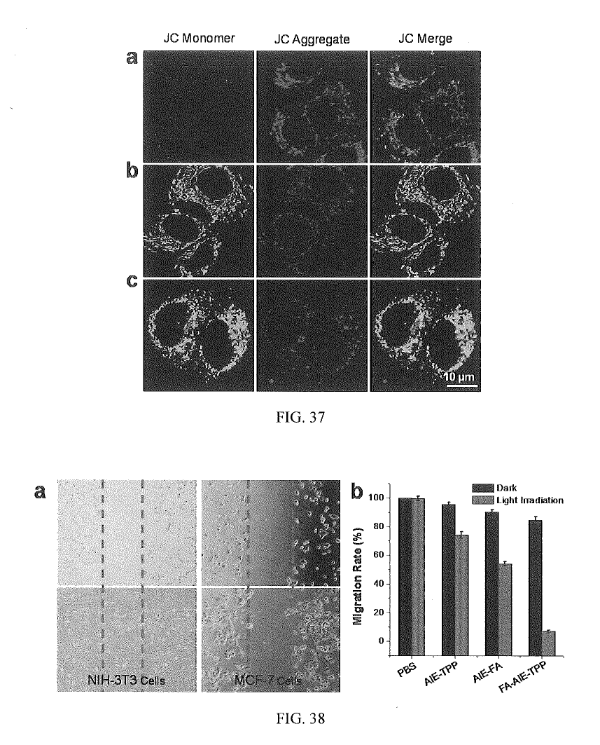

FIG. 37 illustrates mitochondria potential changes of FA-AIE-TPP dots treated MCF-7 cancer cells measured by JC 1 after light irradiation for a) 0, b) 5, and c) 10 min. All the images share the same scale bar.

PDT treatment on mitochondria can cause mitochondria damage, leading to cell apoptosis and death. One of the particular phenomena of mitochondria damage or dysfunction is the loss of mitochondria membrane potential (MMP), which will trigger the release of cytochrome at early stage of apoptosis. A membrane-permeable JC-1 dye to monitor MMPs changes during PDT treatment was used. JC-1 dye undergoes reversible fluorescence changes between its aggregate and monomer states. At high MMP level, JC-1 forms red emissive fluorescent aggregates on normal mitochondria, while it is shifted to green emissive monomer on depolarized mitochondria with low MMP. FIG. 37 shows the representative confocal images of JC-1 assays, and the green/red (G/R) ratio helps quantify the MMP loss of MCF-7 cells during PDT. The accumulation of FA-AIE-TPP dots in mitochondria in dark does not de-polarize the mitochondria membrane as evidenced by the dim green fluorescence and bright red fluorescence from JC-1 dye. Upon exposure to white light, the JC-1 staining changes, where green fluorescence increases at the expense of red fluorescence (G/R ratio changes from 0.46 to 3.59 and 4.37), indicating the loss of MMPs and damage of mitochondrial upon light irradiation. It should be noted that the red fluorescence emitted from FA-AIE-TPP dots is still observable during PDT treatment, which provides the opportunity to visualize the morphology changes of mitochondria from characteristic tubular-like structure to dot-like structures after light irradiation.

FIG. 38 illustrates a) White field image of FA-AIE-TPP dots treated NIH-3T3 and MCF-7 Cells before (up) and after 72 h culture (bottom). Cells were incubated with FA-AIE-TPP dots (20 μg/mL based on DPBA-TPE mass concentration) for 4 h, followed by light exposure (100 mW/cm²) for 10 min. b) The effects of AIE dots treatment on migration of MCF-7 cells with and without light irradiation.

As the powerhouse of cells, mitochondrion provides the major energy for cancer cell activities, including proliferation, migration and metastasis. It is postulated, but not intended to be limited to the theory that, the dysfunction of mitochondria highly affects the ATP production and hence the migration of cancer cells. A cell-scratch spatula method is used to study the effects of AIE dots on cell migration before and after light irradiation. A scratch was applied to the cell monolayer prior to 4 h incubation with these three AIE dots (20 μg/mL based on DPBA-TPE mass concentration) and light irradiation (100 mWcm⁻², 10 min). The migration ratio is determined by the number of cells migrated to the wound area after PDT treatment to that of control cells without AIE dots treatment and light irradiation after 72 h post-culture (FIG. 38). The AIE dots and light irradiation did not affect the migration ability of NIH-3T3 cells, as NIH-3T3 cells migrated into the wound area with a very high migration ratio of ˜100%. On the other hand, AIE dots in dark do not affect the migration ability of MCF-7 cells, but further light irradiation inhibited the wound closure of AIE dots treated MCF-7 cells, with migration ratios of 74.2%, 54.1%, and 6.8% for AIE-TPP, AIE-FA and FA-AIE-TPP dots, respectively (FIG. 38b). As cancer cells are highly metastatic, the inhibition of migration should also contribute to the anticancer therapy.

### REFERENCES

1. Zhao, Z., W. Y. Lam, J., & Zhong Tang, B., Aggregation-Induced Emission of Tetraarylethene Luminogens. Current Organic Chemistry, 14, 2109-2132 (2010).
2. Li, K. et al., Folic acid-functionalized two-photon absorbing nanoparticles for targeted MCF-7 cancer cell imaging. Chemical Communications, 47, 7323 (2011).
3. Li, K. et al., Organic Dots with Aggregation-Induced Emission (AIE Dots) Characteristics for Dual-Color Cell Tracing. Chemistry of Materials, 25, 4181-4187 (2013).
4. Ding, D. et al., Ultrabright Organic Dots with Aggregation-Induced Emission Characteristics for Real-Time Two-Photon Intravital Vasculature Imaging. Advanced Materials, 25, 6083-6088 (2013).
5. Li, K. et al., Biocompatible organic dots with aggregation-induced emission for in vitro and in vivo fluorescence imaging. Science China Chemistry, 56, 1228-1233 (2013).
6. Geng, J. et al., Eccentric Loading of Fluorogen with Aggregation-Induced Emission in PLGA Matrix Increases Nanoparticle Fluorescence Quantum Yield for Targeted Cellular Imaging. Small, 9, 2012-2019 (2013).
7. Li, K. et al., Gadolinium-Functionalized Aggregation-Induced Emission Dots as Dual-Modality Probes for Cancer Metastasis Study. Advanced Healthcare Materials, 2, 1600-1605 (2013).
8. Li, K. et al., Photostable fluorescent organic dots with aggregation-induced emission (AIE dots) for noninvasive long-term cell tracing. Sci. Rep., 3, (2013).
9. D. Ding, K. Li, B. Liu, B. Z. Tang, Acc. Chem. Res. 2013, 46, 2441-2453.
10. J. Wu, W. Liu, J. Ge, H. Zhang, P. Wang, Chem. Soc. Rev. 2011, 40, 3483.
11. Z. Chi, X. Zhang, B. Xu, X. Zhou, C. Ma, Y. Zhang, S. Liu, J. Xu, Chem. Soc. Rev. 2012, 41, 3878.
12. M. Wang, G. Zhang, D. Zhang, D. Zhu, B. Z. Tang, J. Mater. Chem. 2010, 20, 1858.
13. Z. Wang, S. Chen, J. W. Y. Lam, W. Qin, R. T. K. Kwok, N. Xie, Q. Hu, B. Z. Tang, J. Am. Chem. Soc. 2013, 135, 8238-8245.
14. H. Shi, R T. K. Kwok, J. Liu, B. Xing, B. Z. Tang, B. Liu, J. Am. Chem. Soc. 2012, 134, 17972-17981.
15. C. W. T. Leung, Y. Hong, S. Chen, E. Zhao, J. W. Y. Lam, B. Z. Tang, J. Am. Chem. Soc. 2013, 135, 62-65.
16. C. Li, T. Wu, C. Hong, G. Zhang, S. Liu, Angew. Chem. Int. Ed. 2012, 51, 455-459.
17. Yuan, Y.; Kwok, R. T. K.; Tang, B. Z.; Liu, B. J. Am. Chem. Soc. 2014, 136, 2546.
18. X. Xue, et al., Adv. Mater. 2014, 26, 712-717.
19. A. Duarte, K. Y. Pu, B. Liu, G. C. Bazan, Chem. Mater. 2010, 23, 501-515
20. L. Feng, C. Zhu, H. Yuan, L. Liu, F. Lv, S. Wang, Chem. Soc. Rev. 2013, 42, 6620.
21. K. Y. Pu, B. Liu, Adv. Funct. Mater. 2011, 21, 3408.
22. J. Liang, K. Li, B. Liu, Chem. Sci. 2013, 4, 1377.
23. H. Jiang, P. Taranekar, J. R. Reynolds, K. S. Schanze, Angew. Chem. 2009, 121, 4364.
24. T. S. Corbitt; J. R. Sommer; S. Chemburu; K. Ogawa; L. K. Ista; G. P. Lopez; D. G. Whitten; K. S. Schanze. ACS Appl. Mater. Interfaces 2009, 1, 48.
25. H. Yuan, H. Chong, B. Wang, C. Zhu, L. Liu, Q. Yang, F. Lv, S. Wang, J. Am. Chem. Soc. 2012, 134, 13184.
26. C. Zhu, Q. Yang, F. Lv, L. Liu, S. Wang, Adv. Mater. 2013, 25, 1203.
27. D. S. Wilson, G. Dalmasso, L. Wang, S. V. Sitaraman, D. Merlin, N. Murthy, Nat. Mater. 2010, 9, 923.
28. M. S. Shim, Y. Xia, Angew. Chem. 2013, 125, 7064; Angew. Chem. Int. Ed. 2013, 52, 6926.
29. U. Hersel, C. Dahmen and H. Kessler, Biomaterials 24 (24), 4385-4415 (2003).
30. Yuan, Y. Y.; Feng, G. X.; Qin, W.; Tang, B. Z.; Liu, B. Chem. Commun. 2014, 50, 8757.
31. Zhao, E. G.; Deng, H. Q.; Chen, S. J.; Hong, Y. N.; Leung, C. W. T.; Lam, J. W. Y.; Tang, B. Z. Chem. Commun. 2014, 50, 14451.
32. S. H. Lee, M. K. Gupta, J. B. Bang, H. Bae, H. J. Sung, Adv. Healthc. Mater. 2013, 2, 908.
33. Moses. B.; You, Y. Med. Chem. 2013, 3, 192.
34. Lee, S. H.; Gupta, M. K.; Bang, J. B.; Bae, H.; Sung, H. J. Adv. Healthc. Mater. 2013, 2, 908.
35. Fischer, D.; Bieber, T. Li, Y. X.; Elsasser, H. P.; Kissel, T. Pharm. Res. 1999, 16, 1273.
36. Qin, A. J.; Lam, J. W. Y.; Tang, L.; Jim, C. K. W.; Zhao, H.; Sun, J. Z.; Tang, B. Z. Macromolecules 2009, 42, 1421.
37. Zhao, L.; Wientjes, M. G.; An, J. L. S. Clin. Cancer Res. 2004, 10, 7994.
38. Z. Demko, K. Sharpless, Org. Lett. 2001, 3, 4091-4094.
39. R. A. Copeland, Enzymes: A Practical Introduction to Structure, Mechanism, and Data Analysis, WILEY-VCH, New York, 2000.
40. L. Bourret S. Thibaut, A. Briffaud, N. Rousset, S. Eleouet, Y. Lajat, T. Patrice, J. Photochem. Photobiol. B 2002, 67, 23-31.
41. T. M. Sun, J. Z. Du, Y. D. Yao, C. Q. Mao, S. Dou, S. Y. Huang, P. Z. Zhang, K. W. Leong, E. W. Song, J. Wang, ACS Nano 2011, 5, 1483-1494.
42. L. Z. Feng, K. Y. Li, X. Z. Shi, M. Gao, J. Liu, Z. Liu, Adv. Healthc. Mater. 2014, 3, 1261-1271.
43. A. P. Castano, P. Mroz, M. X. Wu, M. R. Hamblin, Proc. Natl. Acad. Sci. U.S.A. 2008, 105, 5495-5500.
44. D. E. Dolmans, D. Fukumura, R. K. Jain, Nat. Rev. Cancer 2003, 3, 380-387.
45. Gomes, A., Fernandes, E., Lima, J. J. Biochem. Biophys. Methods 2005, 65, 45.
46. Shi, H.; Liu, J.; Geng, J.; Tang, B. Z.; Liu, B. Journal of the American Chemical Society 2012, 134, 9569-9572.
47. Shi, H.; Kwok, R. T. K.; Liu, J.; Xing, B.; Tang, B. Z.; Liu, B. Journal of the American Chemical Society 2012, 134, 17972-17981.
48. Shi, H.; Zhao, N.; Ding, D.; Liang, J.; Tang, B. Z.; Liu, B. Organic & Biomolecular Chemistry 2013, 11, 7289-7296.
49. Huang, Y.; Hu, F.; Zhao, R.; Zhang, G.; Yang, H.; Zhang, D. Chemistry—A European Journal 2014, 20, 158-164.
50. Zhang, Y.; Chen, J.; Zhang, Y. Hu, Z.; Hu, D.; Pan, Y.; Ou, S.; Liu, G.; Yin, X.; Zhao, J.; Ren, L.; Wang, J. Journal of Biomolecular Screening 2007, 12, 429-435.
51. Ding, D., Li, K., Liu, B. & Tang, B. Z. Bioprobes Based on AIE Fluorogens. Accounts Chem Res 46, 2441-2453, doi:Doi 10.1021/Ar3003464 (2013).
52. Y. Yuan, C. J. Zhang, M. Gao, R Zhang, B. Z. Tang, B. Liu, Angew. Chem. Int. Ed., 54(6): 1780-86 (2015).
53. R. a J. Smith, R. C. Hartley and M. P. Murphy, Antioxid. Redox Signal., 2011, 15, 3021-3038.
54. F. Hu, Y. Huang, G. Zhang, R. Zhao, H. Yang and D. Zhang, Anal. Chem., 2014, 86, 7987-7995.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A fluorophore having the structure of Formula (XI): 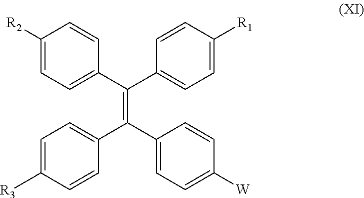
or a pharmaceutically acceptable salt thereof;
wherein W is a conjugated system;
R₁ and R₂ are H, OH, N(C₁-C₃)alkyl or O(C₁-C₆) alkyl optionally substituted with one or more substituents selected from halo, amino, PPh₃, 5-10 atom heterocycyl, N₃, —C(O)(C₂-C₆)alkynyl or X;
R₃ is H, OH, N(C₁-C₃)alkyl or O(C₁-C₆) alkyl optionally substituted with one or more substituents selected from halo, amino, PPh₃, 5-10 atom heterocycyl, N₃, —C(O)(C₂-C₆)alkynyl, X or W;
X is a moiety comprising a linking moiety, a plurality of hydrophilic peptides, a target recognition motif and optionally TPE2; and
the fluorophore exhibits aggregation-induced emission properties.

2. The fluorophore of claim 1, wherein the conjugated system comprises one or more aromatic rings, one or more heteroaromatic rings, one or more alkenes, one or more heteroatoms comprising a p-orbital, or a combination thereof.

3. The fluorophore of claim 1, wherein the conjugated system is: 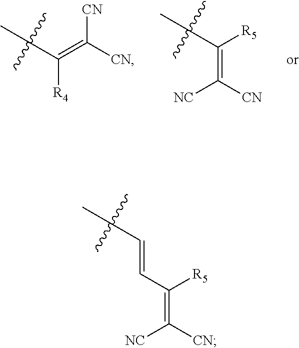
R₄ is (C₁-C₆) alkyl optionally substituted with N₃, amino, (C₁-C₃)alkynyl, —C(O)OH, halo, —SH, maleimide or OH;
R₅ is aryl, heteroaryl, (C₁-C₆) alkyl or (C₂-C₆) alkenyl optionally substituted with N₃, amino, (C₁-C₃)alkynyl, —C(O)OH, halo, —SH, maleimide, OH, aryl or heteroaryl, each further optionally substituted with —O—(C₁-C₆) alkylamino; and
R₆ is aryl or heteroaryl.

4. The fluorophore of claim 1, wherein the linking moiety the linking moiety comprises a chemical bond that breaks upon exposure to an external stimulus.

5. The fluorophore of claim 1, wherein the linker is

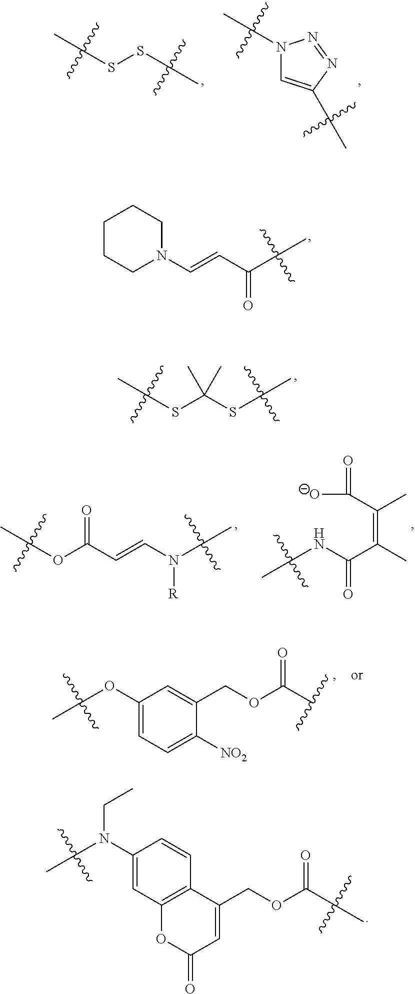

6.-12. (canceled)

13. The fluorophore of claim 1, wherein the fluorophore is encapsulated into a biocompatible matrix;
wherein the matrix comprises lipids, polyethylene glycol, chitosan, polyvinyl alcohol, poly(2-hydroxyethylmethacrylate) or bovine serum albumin;
wherein polyethylene glycol, chitosan, polyvinyl alcohol, poly(2-hydroxyethylmethacrylate) or bovine serum albumin is optionally functionalized by one or more lipids, maleimide, hydroxyl, amine, carboxyl, sulfhydryl or a combination thereof.

14. The fluorophore of claim 13, wherein an outer surface of the biocompatible matrix is functionalized with a cell penetrating peptide comprising an amino acid residue sequence of Arg-Lys-Lys-Arg-Arg-Gln-Arg-Arg-Arg-Cys (SEQ ID NO: 1), Arg-Arg-Arg-Arg-Arg-Arg-Arg-Arg (SEQ ID NO: 2), Lys-Arg-Pro-Ala-Ala-Thr-Lys-Lys-Ala-Gly-Gln-Ala-Lys-Lys-Lys-Leu (SEQ ID NO: 3), and Gly-Leu-Ala-Phe-Leu-Gly-Phe-Leu-Gly-Ala-Ala-Gly-Ser-Thr-Met-Gly-Ala-Trp-Ser-Gln-Pro-Lys-Lys-Lys-Arg-Lys-Val (SEQ ID NO: 4), or Val-His-Leu-Gly-Tyr-Ala-Thr (SEQ ID NO: 8), or a pharmaceutically acceptable salt thereof.

15. A method for visualization of a biological object, comprising:
incubating a biological sample containing the biologic object to be visualized with the fluorophore of claim 1 under conditions sufficient to form an incubated mixture;
irradiating the incubated mixture; and
visualizing the irradiated mixture by fluorescence.

16. A chemical composition, comprising:
a target recognition motif, a fluorophore, a linking moiety and a chemotherapeutic drug, wherein the target recognition motif, the fluorophore, the linking moiety and the chemotherapeutic drug are linked by covalent linkages in a linear array;
the target recognition motif is at a terminal end of the linear array; and
further wherein the fluorophore exhibits aggregation-induced emission properties and comprises a tetraphenylethylene optionally substituted with H, OH, O(C₁-C₆)alkyl, aryl, heteroaryl, or (C₂-C₆) alkenyl further optionally substituted with —CN.

17.-23. (canceled)

24. A method for assessing the conversion of a prodrug into its active form, comprising:
a) incubating a biological sample with a composition of claim 16 under conditions sufficient to form an incubated mixture; and
b) analyzing the fluorescence of the incubated mixture of step a), wherein a change in fluorescence signal as compared to the fluorescence signal of the composition not in the presence of the biological sample is indicative of the conversion of the prodrug into its active form.

25.-26. (canceled)

27. A conjugated polymer of Formula (V): 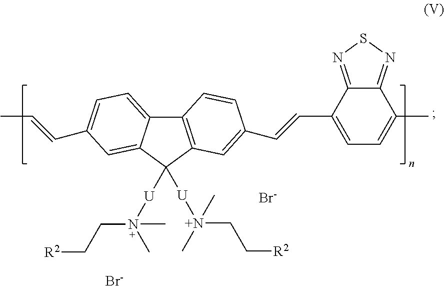
or a salt thereof, wherein:
U is (C₁-C₂₀)alkyl or (CH₂CH₂O)₁₋₂₀;
R² is 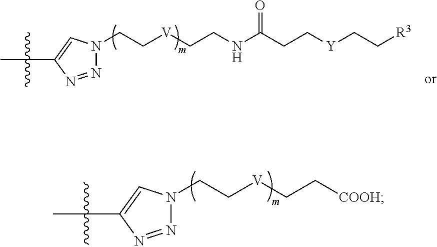
V is O or NH or Si;
Y is 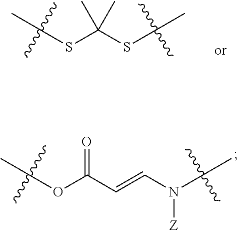
Z is H or (C₁-C₆)alkyl;
each R³ is independently —COOH or —CO—B;
B is a chemotherapeutic drug;
n is an integer from 5-115; and
m is an integer from 5-115.

28. The conjugated polymer of claim 27, wherein at least one R³ is —CO—B.

29. The conjugated polymer of claim 27, wherein the chemotherapeutic drug is doxorubicin, paclitaxel, melphalan, camptothecin, or gemcitabine.

30.-33. (canceled)

34. A method for the treatment of cancer through combination chemotherapy and photodynamic therapy, comprising:
a) incubating a biological sample thought to contain cancer cells with the conjugated polymer-based nanoparticle of claim 27 under conditions sufficient to form an incubated mixture, wherein at least one R³ is —CO—B; and
b) irradiating the incubated mixture with a light of a wavelength sufficient to generate a reactive oxygen species, wherein the reactive oxygen species reacts with the conjugated polymer to convert the chemotherapeutic drug into an active form and further wherein the reactive oxygen species activates the conjugated polymer to serve as a photosensitizer.

35. The method of claim 34, further comprising visualizing the irradiated mixture by fluorescence, wherein a change in fluorescence signal of the irradiated mixture, as compared to the fluorescence signal of the conjugated polymer-based nanoparticle prior to incubation is indicative of conversion of the chemotherapeutic drug into an active form.

36.-52. (canceled)

53. A polymer comprising a fluorophore of claim 1, a linking moiety and an oligoethylenimine or plurality of peptides, wherein the fluorophore, the linking moiety and the oligoethylenimine or plurality of peptides are linked by covalent linkages in a linear array; and
further wherein the fluorophore exhibits aggregation-induced emission properties and comprises a tetraphenylethylene optionally substituted with H, OH, O(C₁-C₆)alkyl, aryl, heteroaryl, or (C₂-C₆) alkenyl further optionally substituted with —CN.

54. The polymer of claim 53, having the structure of Formula (XII) 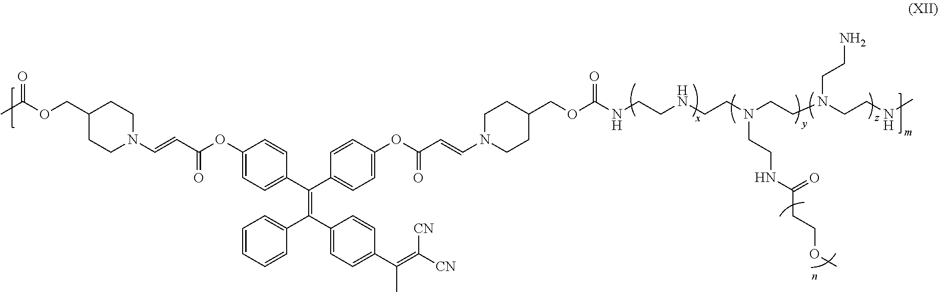
wherein m is an integer between 1 and 200, n is an integer between 5 and 400, and x+y+z is an integer between 5 and 10.

55. A method of delivering a target agent to a cell, the method comprising:
a) contacting the polymer of claim 53 with the target agent under conditions sufficient to form an agent-polymer particle;
b) incubating the cell with the agent-polymer particle under conditions sufficient to form an incubated mixture; and
c) irradiating the incubated mixture with a light of a wavelength sufficient to generate a reactive oxygen species, wherein the reactive oxygen species reacts with the agent-polymer particle to release the agent from the agent-polymer particle into the cell.

56. The method of claim 55, wherein the agent is DNA, RNA, SiRNA, or a drug.

57. A method for designing and screening a photosensitizer compound of claim 1 for photodynamic therapy, comprising:
a) selecting a class of compounds comprising a donor moiety and an acceptor moiety;
b) calculating, for a plurality of members of the class of compounds, values of the energy gap between the singlet and triplet excited states (ΔE_{ST});
c) identifying members of the class of compounds with ΔE_{ST} less than or equal to 1;
d) photoexciting the identified members of the class of compounds to generate singlet oxygen; and
e) selecting the photosensitizer compound from the compounds of step (d) with the highest singlet oxygen quantum yield.


SEQUENCE LISTING (8 sequences)

SEQ ID NO: 1 - protein, 10 aa, Artificial Sequence
  Artificially Synthesized Protein
RKKRRQRRRC

SEQ ID NO: 2 - protein, 8 aa, Artificial Sequence
  Artificially Synthesized Protein
RRRRRRRR

SEQ ID NO: 3 - protein, 16 aa, Artificial Sequence
  Artificially Synthesized Protein
KRPAATKKAGQAKKKL

SEQ ID NO: 4 - protein, 27 aa, Artificial Sequence
  Artificially Synthesized Protein
GLAFLGFLGAAGSTMGAWSQPKKKRKV

SEQ ID NO: 5 - protein, 10 aa, Artificial Sequence
  Artificially Synthesized Protein
GRKKRRQRRR

SEQ ID NO: 6 - protein, 8 aa, Artificial Sequence
  Artificially Synthesized Protein
GVHLGYAT

SEQ ID NO: 7 - protein, 12 aa, Artificial Sequence
  Artificially Synthesized Protein
DDDDDVHLGYAT

SEQ ID NO: 8 - protein, 7 aa, Artificial Sequence
  Artificially Synthesized Protein
VHLGYAT